(19) United States
(12) Patent Application Publication
(10) Pub. No.: US 2003/0232354 A1
(43) Pub. Date: Dec. 18, 2003

(54) Nucleic acid reactions using labels with different redox potentials

(76) Inventors: Changjun Yu, Pasadena, CA (US); Yitzhak Tor, San Diego, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/336,225

(22) Filed: Jan. 2, 2003

Related U.S. Application Data
(63) Continuation of application No. 10/116,726, filed on Apr. 3, 2002, now abandoned.
(63) Continuation of application No. 09/626,096, filed on Jul. 26, 2000.
(60) Provisional application No. 60/281,276, filed on Apr. 3, 2001.

(51) Int. Cl. C12Q 1/68; C07H 21/04

(52) U.S. Cl. 435/6; 536/24.3

(57) ABSTRACT

The present invention is directed to methods and compositions for the use of electron transfer moieties with different redox potentials to electronically detect nucleic acids, particularly for the electrochemical sequencing of DNA.

This is a continuing application of No. 60/281,276, filed Apr. 3, 2001 and Ser. No. 09/626,096, filed Jul. 26, 2000.

### FIELD OF THE INVENTION

The present invention is directed to methods and compositions for the use of electron transfer moieties with different redox potentials to electronically detect nucleic acids, particularly for the electrochemical sequencing of DNA.

### BACKGROUND OF THE INVENTION

DNA sequencing is a crucial technology in biology today, as the rapid sequencing of genomes, including the human genome, is both a significant goal and a significant hurdle. Traditionally, the most common method of DNA sequencing has been based on polyacrylamide gel fractionation to resolve a population of chain-terminated fragments (Sanger et al., Proc. Natl. Acad. Sci. USA 74:5463 (1977); Maxam & Gilbert). The population of fragments, terminated at each position in the DNA sequence, can be generated in a number of ways. Typically, DNA polymerase is used to incorporate dideoxynucleotides that serve as chain terminators.

Several alternative methods have been developed to increase the speed and ease of DNA sequencing. For example, sequencing by hybridization has been described (Drmanac et al., Genomics 4:114 (1989); Koster et al., Nature Biotechnology 14:1123 (1996); U.S. Pat. Nos. 5,525,464; 5,202,231 and 5,695,940, among others). Similarly, sequencing by synthesis is an alternative to gel-based sequencing. These methods add and read only one base (or at most a few bases, typically of the same type) prior to polymerization of the next base. This can be referred to as “time resolved” sequencing, to contrast from “gel-resolved” sequencing. Sequencing by synthesis has been described in U.S. Pat. No. 4,971,903 and Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996), Nyren et al., Anal. Biochem. 151:504 (1985). Detection of ATP sulfurylase activity is described in Karamohamed and Nyren, Anal. Biochem. 271:81 (1999). Sequencing using reversible chain terminating nucleotides is described in U.S. Pat. Nos. 5,902,723 and 5,547,839, and Canard and Arzumanov, Gene 11:1 (1994), and Dyatkina and Arzumanov, Nucleic Acids Symp Ser 18:117 (1987). Reversible chain termination with DNA ligase is described in U.S. Pat. No. 5,403,708. Time resolved sequencing is described in Johnson et al., Anal. Biochem. 136:192 (1984). Single molecule analysis is described in U.S. Pat. No. 5,795,782 and Elgen and Rigler, Proc. Natl. Acad Sci USA 91(13):5740 (1994). Sequencing using mass spectrometry techniques is described in Koster et al., Nature Biotechnology 14:1123 (1996); Krahmer, et al., Anal. Chem., 72:4033 (2000), all of which are hereby expressly incorporated by reference in their entirety.

Other means for improving sequencing rates include capillary electrophoresis. Capillary Electrophoresis (CE) is proving to be a powerful tool for DNA-sequencing and fragment sizing due to its low sample volume requirements, higher efficiency and rapidity of separations compared to the traditional approach of slab gel electrophoresis (Swerdlow, H. and Gesteland, R., (1990) Nucl. Acid. Res. 18, 1415-1419) (Kheterpal, I., Scherer, J. R., Clark, S. M., Radhakrishnan, A., Ju. J., Ginther, C. L., Sensabaugh, G. F. and Mathies, R. A., (1996) Electrophoresis 17, 1852-1859). More recently, microfabricated CE devices and Capillary Array Electrophoresis (CAE) microplates have demonstrated their potential for rapid, parallel separation of DNA sizing and sequencing samples (Woolley, A. T. and Mathies, R. A., (1994) Proc. Natl. Acad. Sci. U.S.A. 91, 11348-11352) (Woolley, A. T. and Mathies, R. A., Anal. Chem. 67, 3676-3680, 1995) (Woolley, A. T., Sensabaugh, G. F., and Mathies, R. A., (1997) Anal. Chem. 69, 2256-2261) (Simpson, P. C., Roach, D., Woolley, A T, Thorsen, T., Johnston, R., Sensabaugh, G. F. and Mathies, R. A., (1998) Proc. Natl. Acad. Sci. U.S.A. 95, 2256-2261).

Fluorescent and electrochemical detection systems may be used in combination with capillary electrophoresis for the detection of DNA sequencing ladders; see Gozel et al., Anal. Chem., 59: 44 (1987); Wu et al., J. Chromatogr., 480: 141 (1989); Smith et al., Nature, 321: 674 (1986); Smith et al., Methods Enzymol., 155: 260 (1987); Park et al., Anal., Chem., 67: 911 (1995); Osbourn et al., Anal. Chem., 73: 5961 (2001); Woods et al., Anal. Chem., 73: 3687 (2001); Ewing et al., Anal., Chem., 66: 52 (1994); Brazill et al., Anal Chem., 73: 4882 (2201); and U.S. Pat. No. 5,244,560; all of which are hereby expressly incorporated by reference in their entirety.

Brazill, et al. describe a method of electrochemical DNA sequencing using ferrocene derivatives with unique sinusoidal voltammetry frequency responses (Brazill, et al., Anal. Chem., 73: 4882 (2001). However, small differences in redox potential between the ferrocene tags makes it difficult to obtain the resolution necessary to increase throughput and sensitivity of this approach. Thus, there still exists a need for an electrochemical sequencing system with increased throughput and sensitivity.

Accordingly, it is an object of the present invention to provide electrochemical methods for determining the sequence of nucleic acids.

### SUMMARY OF THE INVENTION

In accordance with the objects outlined above, the present invention provides compositions comprising nucleic acids comprising ETMs with unique redox potentials. Thus, the present invention provides compositions comprising a first nucleic acid comprising a first ETM with a first redox potential, a second nucleic acid comprising a second ETM with a second redox potential, a third nucleic acid comprising a third ETM with a third redox potential, and a fourth nucleic acid comprising a fourth ETM with a fourth redox potential. The first, second, third, and fourth redox potentials are different. The sequences of the nucleic acids can be the same or different, and in a preferred embodiment, they differ by at least one base. The compositions may further comprise additional nucleic acids, also with unique redox potentials. Preferably, the ETMs are transition metal complexes that can be tuned via chemical substitutents to have unique and non-overlapping redox potential.

In a further aspect, the invention provides methods of sequencing comprising providing a plurality of sequencing probes complementary to a target sequence, wherein each sequencing probe is of a different length and comprises a different chain terminating nucleic acid analog comprising an ETM with a different redox potential. The population of sequencing probes can be separated on the basis of size and the detection of the ETM used to identify the sequence of the target nucleic acid.

In an additional aspect, the methods are directed to methods of determining the identification of a nucleotide at a detection position in a target sequence. The target sequence comprises a first target domain directly 5′ adjacent to the detection position. The method comprises providing an assay complex comprising the target sequence, a capture probe covalently attached to an electrode, and an extension primer hybridized to the first target domain of the target sequence. A polymerase enzyme and a plurality of dNTPs each comprising a covalently attached ETM with a unique redox potential are provided, under conditions whereby if one of the dNTPs basepairs with the base at the detection position, the extension primer is extended by the enzyme to incorporate a dNTP comprising an ETM, which is then detected to determine the identity of the base at the detection position.

In an additional aspect, methods of making a plurality of nucleic acids, each with a covalently attached ETM with a different redox potential comprising providing a first transitional metal complex with a first redox potential and a first functional group; providing a first oligonucleotide substituted with a second functional group; mixing said first transition metal complex with said first oligonucleotide to form a first transition metal complex-oligonucleotide conjugate with a first redox potential; providing a second transitional metal complex with a second redox potential and a first functional group; providing a second oligonucleotide substituted with a second functional group; and mixing said second transition metal complex with said second oligonucleotide to form a second transition metal complex-oligonucleotide conjugate with a second redox potential.

### BRIEF DESCRIPTION OF THE DRAWINGS

FIG. 1 depicts the Faradaic current and capacitive.

FIG. 2 depicts the sketch of the fourth harmonic of the Faradaic signal.

FIG. 3 depicts the sketch of the background.

FIG. 4 depicts the third derivative of the Gaussian.

FIG. 5 depicts uncertainty on the 1p estimation for 95% confidence of a 2peak interation.

FIG. 6 depicts Means and Stdev used to henerate the synthetic files.

FIG. 7 depicts peaks found when only 1 P and 4P were present. 0% noise.

FIG. 8 depicts peaks found when only 1 P and 4P were present, 10% noise.

FIG. 9 depicts peaks found when only 1 P and 3P were present.

FIG. 10 depicts peaks found when only 1P and 3P were present. 10% noise.

FIG. 11 depicts 4 potential simulations for various Ips. Noise level=0.1.

FIG. 12 depicts Ip found on experiment WS145.

FIG. 13 depicts the initial guess and constrain parameters used in the code.

FIG. 14 depicts synthesis of alkoxy ferrocene derivatives with mono-alkoxy group.

FIG. 15 depicts synthesis of dialkoxyl groups.

FIGS. 16A-C depicts a mono-halogenated ferrocene derivatives.

FIGS. 17A-B depicts non nucleosidic ferrocene phosphoramidite.

FIGS. 18A-E depicts ferrocenes with high redox potentials.

FIG. 19 depicts ferrocene derivatives for post-synthesis of nucleic acid. FIG. 20 depicts a general structure for electrochemical sequencing.

FIG. 21 depicts a representative retrosynthesis of an electrochemically-active nucleotide.

FIG. 22 depicts a proposed first generation phosphoramidites suitable for 5′-labeling of synthetic DNA primers.

FIG. 23 depicts two major experiments employed to explore the incorporation of the redox-active deoxy- and dideoxynucleotides in comparison to their “native”

counterparts. FIG. 24 depicts various positions suitable for structural modifications without altering the electrochemical propitious of the metal center.

FIG. 25 depicts the first generation electrochemically-distinguished chain terminating didioxynucleoside triphosphates.

FIG. 26 depicts two alternative designs for tunable redox-active centers that can be linked to modified ddNTPs.

FIG. 27 depicts oxidation potential of Ru²⁺ complexes and their tuning.

FIGS. 28A-1 depicts methods of preparing multi-ferrocene analogs.

FIGS. 29A-B illustrates the general synthesis of ferrocene derivatives with oligonucleotides in aqueous or aqueous DMF (or DMSO) to give the desired products.

FIG. 30 illustrates some of the ferrocene derivative of the invention and their redox potential.

FIG. 31 illustrates incorporation of dRuTP by DNA polymerase (klenow fragment).

FIGS. 32A-D illustrates a diagram for electronic detection fo DNA sequencing mixtures.

### DETAILED DESCRIPTION OF THE INVENTION

The present invention is directed to methods of determining the sequence of a target nucleic acid using electrochemical detection on an electrode. The invention includes the use of redox-active DNA labeling agents for the electrochemical detection of nucleic acid oligonucleotides. The redox-active labeling agents are based on electron transfer moieties (“ETMs”), with redox properties that can be tuned to match a range of redox potentials differing by 100 millivolts or more.

These tags can be used in the dideoxy chain termination method developed by Sanger. In this method, four base specific sets of DNA fragments, whose length can be correlated with a specific base positioning are generated. Fragment sizing with single base resolution is utilized to read the sequence. Samples are prepared by primer extension protocols where a short single stranded complementary DNA oligonucleotide, i.e., the primer, is hybridized to a target sequence. Addition of DNA polymerase and a mixture of deoxynucleotide triphosphates (dNTPs) for each of the bases, i.e., A, T, G and C, leads to extension of the double stranded region. Addition of dideoxynucleotide triphosphate (ddNTP) to the mixture results in chain termination at that particular base. By initiating separate reactions with controlled concentrations of ddNTP for each of the four bases, mixtures of nucleic acid fragments terminated at a particular base are generated. Based on the length distribution of the synthesized oligonucleotides in each of the four mixtures, the sequence of the target nucleic acid can be determined.

Thus, the present invention provides compositions and methods of using ETM labeled nucleic acids for determining the sequence of a target nucleic acid. For example, ddNTPs conjugated to ETMs with different redox potentials may be incorporated by an enzyme in a sequencing reaction to generate sequencing probes comprising ETMs with different redox potentials.

Preferably, capillary electrophoresis channels coupled to electrodes are used to detect and identify ETM labeled oligonucleotides. As will be appreciated by those of skill in the art, four sequential electrodes set at four different potentials may be used to determine the sequence of the target nucleic acid. Alternatively, a single electrode may be used to identify the four bases. In this method, the potential is varied to cover the range of potentials of the ETM labels and the resulting signals scanned to determine the sequence of the target nucleic acid.

Accordingly, the present invention provides compositions and methods for determining the sequence of a target nucleic acid in a sample. As will be appreciated by those in the art, the sample solution may comprise any number of things, including, but not limited to, bodily fluids (including, but not limited to, blood, urine, ser um, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification as is generally described in PCT/US99/01705, such as PCR amplification reaction); purified samples, such as purified genomic DNA, RNA, proteins, etc.; raw samples (bacteria, virus, genomic DNA, etc. As will be appreciated by those in the art, virtually any experimental manipulation may have been done on the sample.

By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meieret al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of ETMs, or to increase the stability and half-life of such molecules in physiological environments.

As will be appreciated by those in the art, all of these nucleic acid analogs may find use in the present invention. In addition, mixtures of naturally occurring nucleic acids and analogs can be made; for example, at the site of conductive oligomer or ETM attachment, an analog structure may be used. Alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched base pairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration. This is particularly advantageous in the systems of the present invention, as a reduced salt hybridization solution has a lower Faradaic current than a physiological salt solution (in the range of 150 mM).

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. A preferred embodiment utilizes isocytosine and isoguanine in nucleic acids designed to be complementary to other probes, rather than target sequences, as this reduces non-specific hybridization, as is generally described in U.S. Pat. No. 5,681,702. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

The compositions and methods of the invention are directed to determining the sequence of target sequences. The term “target sequence” or “target nucleic acid” or grammatical equivalents herein means a nucleic acid sequence on a single strand of nucleic acid. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of a reaction such as an extended probe from an SBE reaction. It may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. As is outlined more fully below, probes are made to hybridize to target sequences to determine the presence or absence of the target sequence in a sample. Generally speaking, this term will be understood by those skilled in the art.

The target sequence may be comprised of different target domains; for example, a first target domain of the sample target sequence may hybridize to a primer, etc. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

As is more fully outlined below, the target sequence comprises a position for which sequence information is desired, generally referred to herein as the “detection position”. In a preferred embodiment, the detection position comprise a plurality of nucleotides, either contiguous with each other or separated by one or more nucleotides. By “plurality” as used herein is meant at least two. In some embodiments, the detection position is a single nucleotide. As used herein, the base which base pairs with the detection position base in a hybrid is termed the “interrogation position”.

In general, current sequencing methods utilize a oligonucleotide primer complementary to a specific sequence on the template strand. As will be appreciated by those of skill in the art, the template strand can be obtained from the target nucleic acid in a variety of ways. For example, the template strand can be obtained as a single-stranded DNA molecule by cloning the target nucleic acid sequence into a bacteriophage M13 or phagemid vector. In addition, the target nucleic acid molecule can be sequenced directly using denatured, double -stranded nucleic acid molecules. In a preferred embodiment, PCR-based methods are used to produce an excess of the target strand that can be used as a template for sequencing.

As will be appreciated by those in the art, a variety of PCR methods can be used, including, but not limited to, asymmetric polymerase chain reaction (APCR), to produce an excess of the target strand.

In a preferred embodiment, asymmetric polymerase chain reaction (APCR) is used to enhance the production of the single stranded nucleic acid fragment used as the template sequence for electrochemical sequencing as outlined herein. Traditional APCR techniques produces a single stranded bias by using the primers in a ratio of 5 to 1, although a variety of ratios ranging from 2:1 to 100:1 can be used as well. See U.S. Ser. No. 09/626,096, filed Jul. 27, 1999 for a description of APCR methods, hereby incorporated by reference in its entirety.

Accordingly, the compositions and methods of the present invention are used to identify the nucleotide(s) at a detection position with the target sequence.

As is more fully outlined below, a variety of ETMs find use in the invention. In this embodiment, the redox potentials of the different ETMs are chosen such that they are distinguishable in the assay system used. By “redox potential” (sometimes referred to as E₀) herein is meant the voltage which must be applied to an electrode (relative to a standard reference electrode such as a normal hydrogen electrode) such that the ratio of oxidized and reduced ETMs is one in the solution near the electrode.

In a preferred embodiment, the redox potentials are separated by at least 100 mV, although differences either less than this or greater than this may also be used, depending on the sensitivity of the system, the electrochemical measuring technique used and the number of different labels used. In a particularly preferred embodiment, derivatives of ferrocene are used; for example, ETMs may be used comprising ferrocene without ring substituents or with the addition of an amine or an amide, a carboxylate, etc.

In a preferred embodiment, the invention provides a plurality of sequencing probes each with at least one ETM with a unique redox potential. By “sequencing probe” herein is meant the population of oligonucleotides generated by the Sanger sequencing reactions. Preferably, each sequencing probe will terminate at a different base and comprise a different covalently attached ETM. Thus, by using four different ddNTPs labeled with an ETM with a unique redox potential, populations of sequencing probes are generated that terminate at positions occupied by every A, C, G, or T in the template strand. These populations can be separated by electrophoresis and the identity of each base determined based on the electrochemical signal of the ETM.

In a preferred embodiment, the identification of the nucleotide at the detection position is done using enzymatic sequencing reactions. Preferably, enzymatic sequencing reactions based on the Sanger dideoxy method and on single base extension are used to determine the identity of the base at the detection position.

In a preferred embodiment, the Sanger dideoxy method is used to determine the identity of the base at the detection position. Briefly, the Sanger method is technique that utilizes primer extension protocols wherein an oligonucleotide primer is annealed to a single stranded DNA template. Four different sequencing reactions are set up each containing a DNA polymerase and dNTPs. The four reactions also include ddNTPs labeled with an ETM as described herein. If a ddNTP molecule is incorporated into a growing DNA chain, further extension of the growing chain is impossible because the absence of the 3′-OH group prevents formation of a phosphodiester bond with the succeeding dNTP. Thus, by including a small amount of one of the labeled ddNTPs with the four dNTPs in a reaction mixture for DNA synthesis, there is competition between extension of the chain and infrequent, but base-specific termination. The products of the reaction are a population of sequencing probes, i.e. oligonucleotides, whose lengths are determined by the distance between the 5′ terminus of the primer used to initiate DNA synthesis and the sites of chain termination. For example, in a sequencing reaction containing ddA, the termination points correspond to all positions normally occupied by a deoxyadenosyl residue. By using the four different ddNTPs in four separate enzymatic reactions, populations of sequencing probes are generated that terminate at positions occupied by every A, C, G, or T in the template strand. These populations can be separated by electrophoresis and the sequence of the newly synthesized strand can be determined by detecting the ETM as described below.

Each sequencing reaction is initiated by introducing the template strand to a solution comprising four unlabelled nucleotide analogs and a chain terminating nucleotide analog comprising an ETM with a unique redox potential. By “nucleotide analog” in this context herein is meant a deoxynucleoside-triphosphate (also called deoxynucleotides or dNTPs, i.e. dATP, dTTP, dCTP and dGTP). By “chain terminating nucleotide analog” herein is meant a dideoxytriphosphate nucleotide or ddNTPs, i.e., ddATP, ddCTP, ddGTP and ddTTP.

In addition to the nucleotide analogs, the solution also comprises an extension enzyme, generally a DNA polymerase. Suitable DNA polymerase include, but are not limited to, the Klenow fragment of DNA polymerase I, a DNA polymerase from Thermus aquaticus (i.e., Taq polymerase), a modified T7 polymerase (i.e., SEQUENASE 1.0 and SEQUENASE 2.0 (U.S. Biochemical)), T5 DNA polymerase and Phi29 DNA polymerase.

In a preferred embodiment, Sanger dideoxy-mediated sequencing reactions are run using a modified T7 DNA polymerase (i.e. Sequenase). In this embodiment, the reaction involves annealing of an extension primer to a complementary strand of the template sequence, a brief polymerization reaction to allow for elongation of the primer and extension and termination reactions to produce a population of sequencing probes. The template may be a denatured double stranded DNA molecule or a single stranded molecule. See Sambrook and Russell, “Molecular Cloning: A Laboratory Manual”, third edition, CSHL Press, New York, 2001, Chapter 12; hereby incorporated by reference in its entirety.

In a preferred embodiment, Sanger dideoxy-mediated sequencing reactions are run using the Klenow fragment of E coli DNA polymerase 1. In this embodiment, the Kienow enzyme is used to sequence single-stranded DNA templates. As discussed above, the reaction involves annealing of an extension primer to a complementary strand of the template sequence, extension and termination reactions to produce a population of sequencing probes. See Sambrook and Russell, “Molecular Cloning: A Laboratory Manual”, third edition, CSHL Press, New York, 2001, Chapter 12; hereby incorporated by reference in its entirety.

In a preferred embodiment, Sanger dideoxy-mediated sequencing reactions are run using Taq DNA polymerase. The steps involved in sequencing with Taq DNA polymerase are similar to those for Sequenase. See Sambrook and Russell, “Molecular Cloning: A Laboratory Manual”, third edition, CSHL Press, New York, 2001, Chapter 12; hereby incorporated by reference in its entirety. In a preferred embodiment, cycle DNA sequencing (also referred to as thermal cycle DNA sequencing or linear amplification DNA sequencing) is used to generate a population of sequencing probes. Cycle DNA sequencing is a sequencing technique that uses asymmetric PCR to generate a single-stranded template for sequencing by the Sanger dideoxy chain termination method(s) described above. In this embodiment, four separate amplification reactions are set up, each containing the same oligonucleotide primer and a different chain terminating ddNTP. Typically, two cycling programs are using. In the first program, reaction mixtures are subjected to 15-40 rounds of conventional thermal cycling. Each amplification cycle consists of three steps: denaturation of the double stranded DNA template, annealing of the extension primer, and then extension of the annealed primer and termination of the extended strand by incorporation of a ddNTP. The resulting partially double-stranded hybrid, comprising a full-length template strand and its complementary chain-terminated product, is denatured during the first step of the next cycle, thereby liberating the template strand for another round of priming, extension, and termination. Thus, the sequencing probes accumulate in a linear fashion during the entire first phase of the cycle-sequencing reaction. In the second cycling program, the annealing step is omitted so that no further extension of primers is possible. Instead, the “chase’ segment provides an opportunity for further extension of reaction products that were not terminated by incorporation of ddNTP during the initial rounds of thermal cycling. See Sambrook and Russell, “Molecular Cloning: A Laboratory Manual”, third edition, CSHL Press, New York, 2001, Chapter 12; hereby incorporated by reference in its entirety.

As will be appreciated by those in the art, the configuration of the Sanger sequencing system can take on several forms. As for the SBE reaction described below, the reaction may be done in solution, and the newly synthesized strands with the base-specific ETM labels detected. For example, the newly synthesized strands may be separated by electrophoresis and the ETM labeled sequencing probes detected as described below.

In a preferred embodiment, electrophoresis is conducted in microcapillary tubes (high performance capillary electrophoresis (HPCE)). One advantage of HPCE is that the heat resulting from the applied electric field is efficiently dissipated due to the high surface area, thus allowing fast separation. The capillary tubes may be part of an electrophoresis module, as is generally described in U.S. Pat. Nos. 5,770,029; 5,126,022; 5,631,337; 5,569,364; 5,750,015, and 5,135,627, and U.S. Ser. No. 09/295,691; all of which are hereby incorporated by reference.

Gel media for separation based on size are known, and include, but are not limited to, polyacrylamide and agarose. One preferred electrophoretic separation matrix is described in U.S. Pat. No. 5,135,627, hereby incorporated by reference, that describes the use of “mosaic matrix”, formed by polymerizing a dispersion of microdomains (“dispersoids”) and a polymeric matrix. This allows enhanced separation of target analytes, particularly nucleic acids. Other polymer materials that may be used in the invention include, but are not limited to, entangled polymers of polyacrylimide (see Ruiz-Martinez, et al., Anal. Chem., 65: 2851 (1993); Zhang, et al., Anal. Chem., 67: 4589 (1995); and Carriho, et al., Anal. Chem., 68: 3305 (1996)), poly(vinylpyrrolidone) (Gao, et al., Anal. Chem., 70: 1382 (1998), poly(ethylene oxide) (Fung et al., Anal Chem., 67: 1913 (1995), and poly(dimethylacrylamide) (Rosenblum, et al., Nucleic Acids Res., 25: 39225 (1997) and Madabhushi, et al., Electrophoresis, 19:224 (1998); all of which are incorporated herein by reference). Similarly, U.S. Pat. No. 5,569,364, hereby incorporated by reference, describes separation media for electrophoresis comprising submicron to above-micron sized cross-linked gel particles that find use in microfluidic systems. U.S. Pat. No. 5,631,337, hereby incorporated by reference, describes the use of thermoreversible hydrogels comprising polyacrylamide backbones with N-substituents that serve to provide hydrogen bonding groups for improved electrophoretic separation. See also U.S. Pat. Nos. 5,061,336 and 5,071,531, directed to methods of casting gels in capillary tubes.

In a preferred embodiment, capillary electrophoresis with integrated electrochemical detection is used to separate the sequencing probes (see Voegel, P. D. & Baldwin, R. P., Electrophoresis, 18: 2267-2278 (1997); Gerhardt, G. C., et al., Anal. Chem., 70: 2167-2173 (1998); Wen, J., et al., Anal., Chem., 70: 2504 (1998); Qian, J., et al., Anal. Chem., 71: 4468 (1999); Woolley, et al., Anal. Chem., 70: 684 (1998); Matysik, F.-M., et al., Anal. Chim. Acta., 385: 409 (1999); all of which are hereby incorporated by reference in their entirety). Preferably, end column detection methods are used to detect ETM labeled probes.

In a preferred embodiment, the ETM labeled probes are detected using end column detection (EC). EC detection has been successfully used as a detection method for capillary electrophoresis in fused-silica capillaries as small as 2 μm in diameter (Olefirowicz, T. M. and Ewing, A. G., (1990) Anal. Chem. 62, 1872-1876), with detection limits for various analytes in the femtomole to attomole mass range. Smaller diameter electrophoretic capillaries require the use of smaller diameter electrodes, or microelectrodes. Background noise is lower at these microelectrodes due to a sharp decrease in background charging currents (Bard, A. J. and Faulkner, L. R., (1980) Electrochemical Methods:Fundamentals and Applications, New York, John Wiley and Sons). This leads to better concentration sensitivity due to the higher signal-to-noise ratio. Mass sensitivity is also enhanced at these microelectrodes over bigger electrodes due to higher coulometric efficiency (Huang, X. H. et al., supra).

In a preferred embodiment, end column detection with electrodes positioned at the outlet(s) of capillary electrophoresis channels is used to detect the ETM labeled probes of the invention.

In a preferred embodiment, ETM labeled probes are detected using end column detection with four electrodes positioned at the outlet of a capillary electrophoresis channel. In this embodiment, the four electrodes are set at different potentials corresponding to the redox potentials of the ETMs. For example, one electrode will be set at a low potential (e.g. −0.1V) sufficient to only oxidize one of the ETM. The next electrode, set at a slightly higher potential (e.g., 0.12V) will be able to oxidize only the two low potential ETMS. The next electrode, set at a slightly higher potential (e.g., 0.27V will be able to oxidize only the three low potential ETMs. The last electrode, set at a slightly higher potential (e.g., 0.5V) will be able to oxidize all four ETMs. Thus, multiple signals will be detected at the higher potential electrodes. Deconvolution using appropriate software will be used to determine the correct sequence. In a preferred embodiment, ETM labeled probes are detected using end column detection with a single electrode positioned at the outlet of a capillary electrophoresis channel. In this embodiment, the potential of the single electrode is varied. For example, a triangle wave can be applied having minimum and maximum potentials that span the potentials of the four ETM labels. For example, if ETMs with −0.1V, 0.12V, 0.27V, and 0.5 V are used, the potential is varied from −0.25V to +0.65V. Deconvolution using appropriate software will be used to determine the correct sequence.

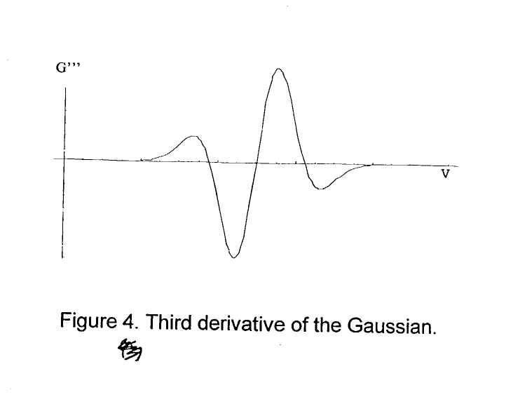

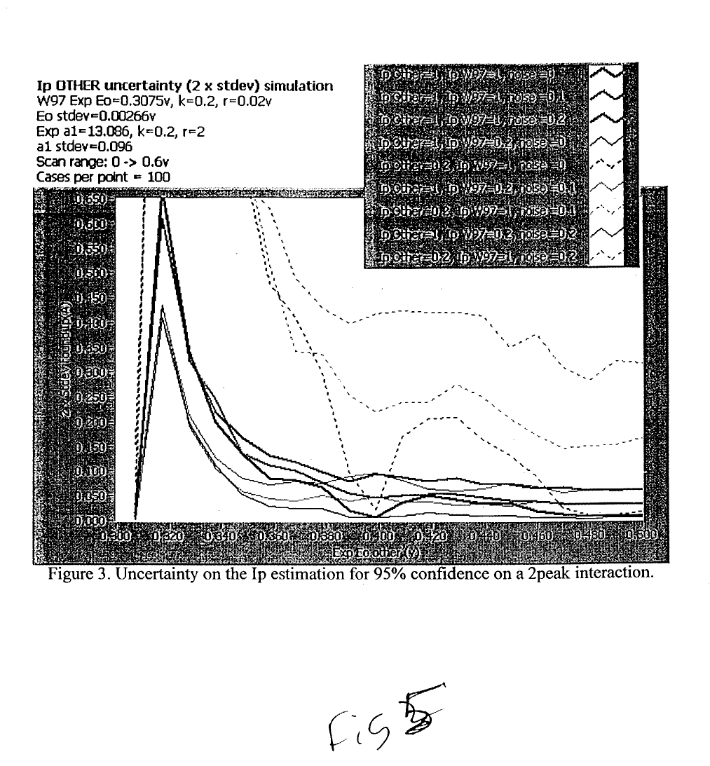

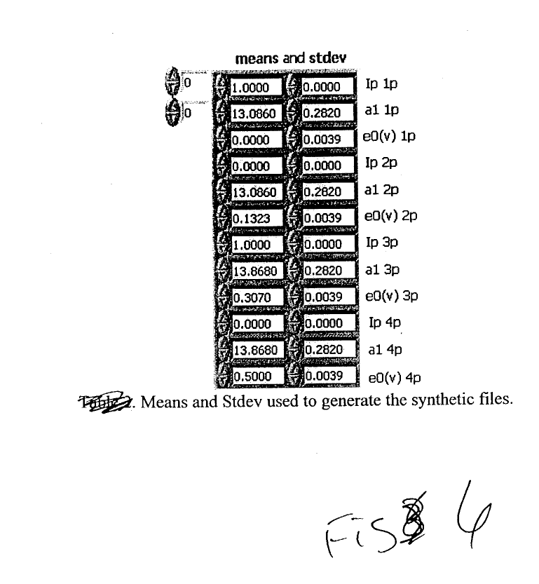

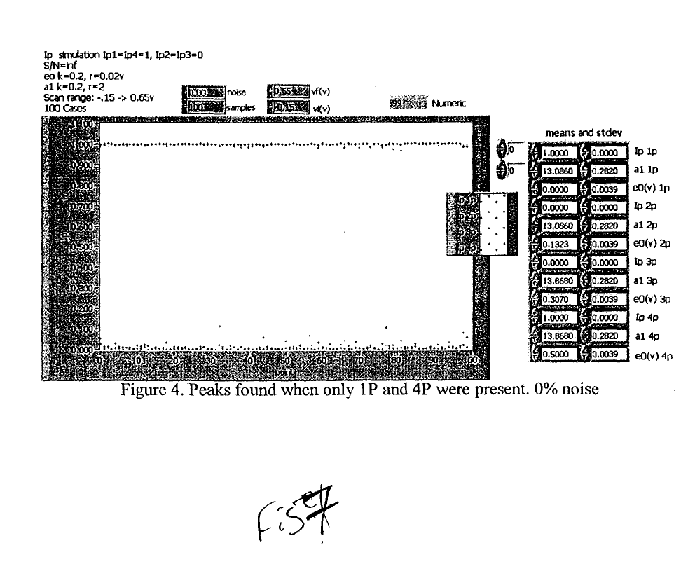

In a preferred embodiment, the faradaic current from ETMs with different redox potentials is quantified using a non-linear regression curve fitting algorithm. The algorithm fits two phases of the voltamogram or the faradaic current previously obtained by a locking process (see Example 1). A function composed by the addition of an arbitrary number, n (i.e., such as the number of ETM labels in the system), of custom made functions that have the same shape as the faradaic signal and another function that describes the background current is fitted to every phase of the voltammogram. One example of such a custom made function is presented in Equation 1. It is composed of a combination of third derivatives of a modified Gaussian distribution (FIG. 4) to simulate the fourth harmonic of the faradaic signal (FIG. 2) and a fifth order polynomial to fit the background current (FIG. 3). For example, the algorithm shown in Equation 1 uses a combination of third derivatives of a modified Gaussian distribution (FIG. 4) to simulate the fourth harmonic of the faradaic signal (FIG. 2) and a fifth order polynomial to fit the background current (FIG. 2).

### Equation 1

$${F}_{i} ( v ) = {\sum }_{n = 1}^{m} {a}_{no} {{ⅇ}^{- {a}_{n1} 2 {( v - {a}_{n2} )}^{2}} ( 3 - 2 {a}_{n1} )}^{2} ( v - {a}_{n2}^{2} ) ( v - {a}_{n2} ) + {P}^{5} ( v ) \qquad ( 1 )$$

This algorithm finds the optimum set of parameters (a₁₀, a₁₁, . . . , aₙ₁, aₙ₂) that define the Gaussian derivatives and the polynomial that minimizes an error coefficient defined in Equation 2.

### Equation 2

$$ϵ = {\sum }_{I} \frac{{( {D}_{i} - {F}_{i} )}^{2}}{{σ}_{i}^{2}} + {\sum }_{n = 1}^{m} {\sum }_{j = 0}^{2} \frac{{{K}_{nj} ( {a}_{nj} - \overline{{a}_{nj}} )}^{2}}{4 {d}_{nj}} \qquad ( 2 )$$

This error coefficient is defined as the sum of the square of the difference between every point in the data and the fitting curve in Equation 1. Additionally, it has a penalty term that increase if the parameters are too different from a set of prescribed expected parameters. The Gaussian “n” of the “m” existent has 3 parameters (i.e., j=0, 1, 2). Thus, if the parameter aₙⱼ is too different from the prescribed expected aₙⱼ, the error coefficient would have a significant contribution, normalized by kₙⱼ and dₙⱼ. This modification of the error coefficient ensures that the functions that fit the ETM labels are centered about the potential value that they signal.

The parameters are found by minimizing the error coefficient. This is done by expanding the gradient of the error coefficient in a Taylor series, and realizing that for a minimum, it has to be zero (Equation 3).

### Equation 3

O=∇ε=∇ε+∇∇εₒ(aₙ−aₙₒ) (3)

or rearranging terms (Equation 4):

### Equation 4

∇∇εₒ(aₙ−aₙₒ)=−∇εₒ (4)

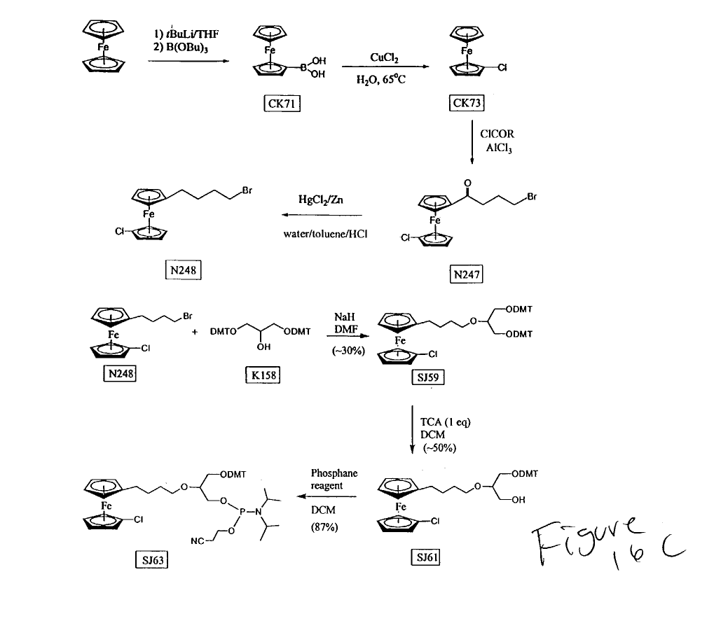

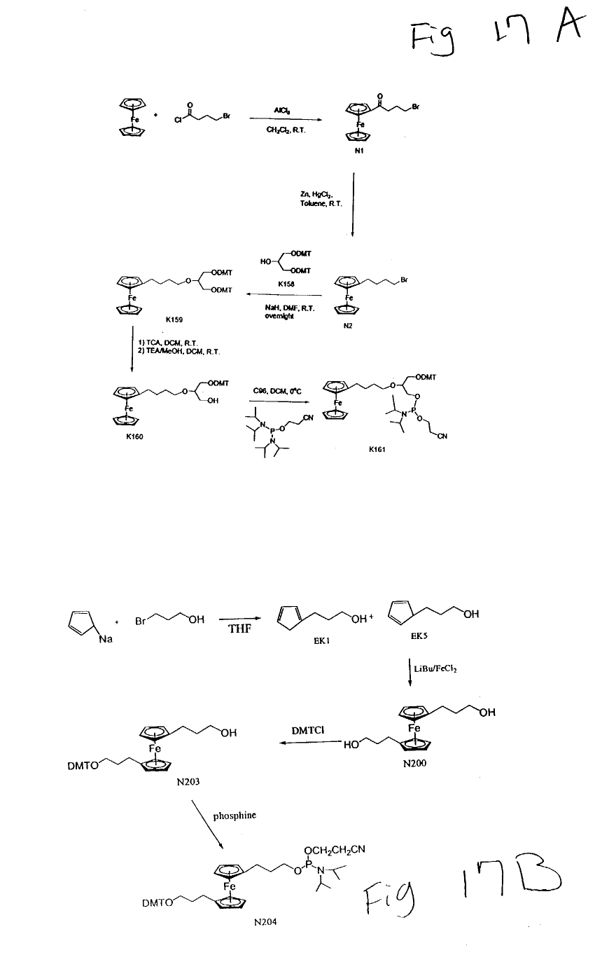

The Marquardt routine puts an additional weight on the diagonal terms, that changes as the algorithm goes, depending on how good the convergence is. The initial guess and constrain parameters used in the code are shown in FIG. 13. Examples 1-4 provide a detailed description of the “peak finder” algorithm and simulations using two and four potential labels.

All of the above compositions and methods are directed to the determination of the identification of the base at one or more detection positions within a target nucleic acid. The detection system of the present invention uses capillary electrophoresis to separate a population of sequencing probes coupled to electrochemical detection of individual sequencing probes containing ETMs with unique redox potentials by passage over one or more electrodes.

In some embodiments, the electrodes may comprise a self-assembled monolayer (SAMs), generally including conductive oligomers. In these embodiments, the composition of the monolayer may be combined with other systems to provide enhanced selectivity or signal amplification (see U.S. Ser. No. 09/626,096, filed Jul. 26, 1999 and U.S. Ser. No. 09/847,113, filed May 1, 2001 for the composition and methods of making and using SAMs; both of which are incorporated herein by reference in their entirety).

Thus, in a preferred embodiment, the compositions comprise an electrode. By “electrode” herein is meant a composition, which, when connected to an electronic device, is able to sense a current or charge and convert it to a signal. Alternatively an electrode can be defined as a composition which can apply a potential to and/or pass electrons to or from species in the solution. Thus, an electrode is an ETM as described herein. Preferred electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite and carbon paste). Preferred electrodes include gold, silicon, carbon and metal oxide electrodes, with gold being particularly preferred.

The electrodes described herein are depicted as a flat surface, which is only one of the possible conformations of the electrode and is for schematic purposes only. The conformation of the electrode will vary with the detection method used. For example, flat planar electrodes may be preferred for optical detection methods, or when arrays of nucleic acids are made, thus requiring addressable locations for both synthesis and detection. Alternatively, for single probe analysis, the electrode may be in the form of a tube, with the SAMs comprising conductive oligomers and nucleic acids bound to the inner surface. This allows a maximum of surface area containing the nucleic acids to be exposed to a small volume of sample.

In a preferred embodiment, the detection electrodes are formed on a substrate. In addition, the discussion herein is generally directed to the formation of gold electrodes, but as will be appreciated by those in the art, other electrodes can be used as well. The substrate can comprise a wide variety of materials, as will be appreciated by those in the art, with printed circuit board (PCB) materials being particularly preferred. Thus, in general, the suitable substrates include, but are not limited to, fiberglass, teflon, ceramics, glass, silicon, mica, plastic (including acrylics, polystyrene and copolymers of styrene and other materials, polypropylene, polyethylene, polybutylene, polycarbonate, polyurethanes, Teflon™, and derivatives thereof, etc.), GETEK (a blend of polypropylene oxide and fiberglass), etc.

In general, preferred materials include printed circuit board materials. Circuit board materials are those that comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer. As is known in the art, one or a plurality of layers may be used, to make either “two dimensional” (e.g. all electrodes and interconnections in a plane) or “three dimensional” (wherein the electrodes are on one surface and the interconnects may go through the board to the other side) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer.

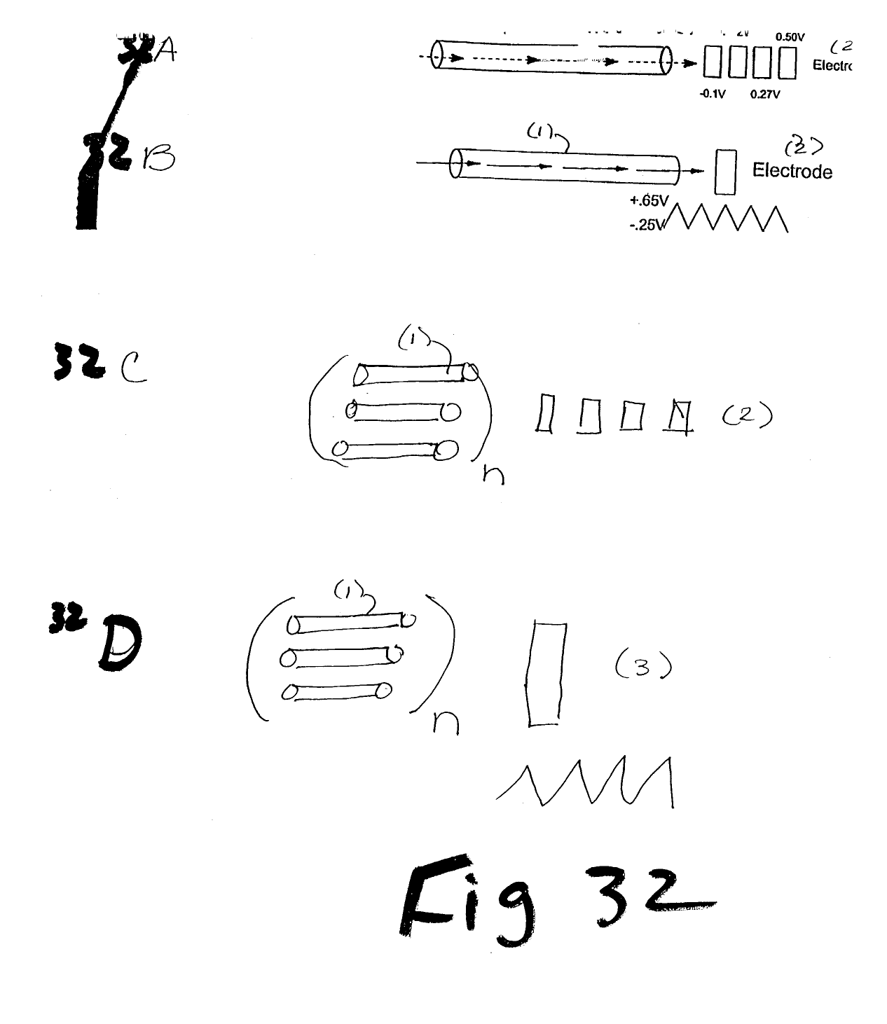

Accordingly, in a preferred embodiment, the present invention provides “sequncing chips” that comprise substrates comprising a plurality of capillary electrophoresis tubes and electrodes. In a preferred embodiment, one or more electrodes is positioned at the outlet of the tube (see FIG. 32A and B). In other embodiments, more than one capillary tube is positioned above one or more electrodes (see FIG. 32C and D).

Regardless of the system, each electrode has an interconnection, that is attached to the electrode at one end and is ultimately attached to a device that can control the electrode. That is, each electrode is independently addressable.

The substrates can be part of a larger device comprising a capillary or gel electrophoresis chamber and a detection chamber or region that exposes a given volume of sample to the detection electrode. Depending on the experimental conditions and assay, smaller volumes may be preferred.

In some embodiments, the electrophoresis chamber, detection chamber and electrode are part of a cartridge that can be placed into a device comprising electronic components (an AC/DC voltage source, an ammeter, a processor, a read-out display, temperature controller, light source, etc.). In this embodiment, the interconnections from each electrode are positioned such that upon insertion of the cartridge into the device, connections between the electrodes and the electronic components are established.

Detection electrodes on circuit board material (or other substrates) are generally prepared in a wide variety of ways. In general, high purity gold is used, and it may be deposited on a surface via vacuum deposition processes (sputtering and evaporation) or solution deposition (electroplating or electroless processes). When electroplating is done, the substrate must initially comprise a conductive material; fiberglass circuit boards are frequently provided with copper foil. Frequently, depending on the substrate, an adhesion layer between the substrate and the gold in order to insure good mechanical stability is used. Thus, preferred embodiments utilize a deposition layer of an adhesion metal such as chromium, titanium, titanium/tungsten, tantalum, nickel or palladium, which can be deposited as above for the gold. When electroplated metal (either the adhesion metal or the electrode metal) is used, grain refining additives, frequently referred to in the trade as brighteners, can optionally be added to alter surface deposition properties. Preferred brighteners are mixtures of organic and inorganic species, with cobalt and nickel being preferred.

In general, the adhesion layer is from about 100 Å thick to about 25 microns (1000 microinches). The If the adhesion metal is electrochemically active, the electrode metal must be coated at a thickness that prevents “bleed-through”; if the adhesion metal is not electrochemically active, the electrode metal may be thinner. Generally, the electrode metal (preferably gold) is deposited at thicknesses ranging from about 500 Å to about 5 microns (200 microinches), with from about 30 microinches to about 50 microinches being preferred. In general, the gold is deposited to make electrodes ranging in size from about 5 microns to about 5 mm in diameter, with about 100 to 250 microns being preferred. The detection electrodes thus formed are then preferably cleaned and SAMs added, as is discussed below.

Thus, the present invention provides methods of making a substrate comprising a plurality of gold electrodes. The methods first comprise coating an adhesion metal, such as nickel or palladium (optionally with brightener), onto the substrate. Electroplating is preferred. The electrode metal, preferably gold, is then coated (again, with electroplating preferred) onto the adhesion metal. Then the patterns of the device, comprising the electrodes and their associated interconnections are made using lithographic techniques, particularly photolithographic techniques as are known in the art, and wet chemical etching. Frequently, a non-conductive chemically resistive insulating material such as solder mask or plastic is laid down using these photolithographic techniques, leaving only the electrodes and a connection point to the leads exposed; the leads themselves are generally coated.

Thus, in a preferred embodiment sequencing probes with attached ETMs are provided. The terms “electron donor moiety”, “electron acceptor moiety”, and “ETMs” (ETMs) or grammatical equivalents herein refers to molecules capable of electron transfer under certain conditions. It is to be understood that electron donor and acceptor capabilities are relative; that is, a molecule which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds in the present invention. Preferred ETMs include, but are not limited to, transition metal complexes, organic ETMs, and electrodes.

In a preferred embodiment, the ETMs are transition metal complexes. Transition metals are those whose atoms have a partial or complete d shell of electrons. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinium (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinium, cobalt and iron.

The transition metals may be complexed with a variety of ligands, L, defined below, to form suitable transition metal complexes, as is well known in the art.

In addition to transition metal complexes, other organic electron donors and acceptors may be covalently attached to the nucleic acid for use in the invention. These organic molecules include, but are not limited to, riboflavin, xanthene dyes, azine dyes, acridine orange, N,N′-dimethyl-2,7-diazapyrenium dichloride (DAP²⁺), methylviologen, ethidium bromide, quinones such as N,N′-dimethylanthra(2,1,9-def:6,5,10-d′e′f′)diisoquinoline dichloride (ADIQ²⁺); porphyrins ([meso-tetrakis(N-methyl-x-pyridinium)porphyrin tetrachloride], varlamine blue B hydrochloride, Bindschedler's green; 2,6-dichloroindophenol, 2,6-dibromophenolindophenol; Brilliant crest blue (3-amino-9-dimethyl-amino-10-methylphenoxyazine chloride), methylene blue; Nile blue A (aminoaphthodiethylaminophenoxazine sulfate), indigo-5,5′,7,7′-tetrasulfonic acid, indigo-5,5′,7-trisulfonic acid; phenosafranine, indigo-5-monosulfonic acid; safranine T; bis(dimethylglyoximato)-iron(II) chloride; induline scarlet, neutral red, anthracene, coronene, pyrene, 9-phenylanthracene, rubrene, binaphthyl, DPA, phenothiazene, fluoranthene, phenanthrene, chrysene, 1,8-diphenyl-1,3,5,7-octatetracene, naphthalene, acenaphthalene, perylene, TMPD and analogs and subsitituted derivatives of these compounds.

In one embodiment, the electron donors and acceptors are redox proteins as are known in the art. However, redox proteins in many embodiments are not preferred.

The choice of the specific ETMs will be influenced by the type of electron transfer detection used, as is generally outlined below. Preferred ETMs are metallocenes, with ferrocene being particularly preferred.

For use in Sanger based sequencing reactions, the ETMs should exhibit several characteristics. First, the redox potentials (i.e., E_{1/2} value) of the ETM should fall outside of the oxidation or reduction potentials of natural heterocyclic bases to provide low background noise and eliminate artifacts. Second, the oxidation or reduction waves of the ETM should be reversible to ensure reproducibility. Third, the ETMs should be chemically stable and compatible with polymerase reaction conditions, PCR amplification and electrophoretic separations. Fourth, the ETM should be “tunable”. By “tunable” herein is meant that the ETM comprises substitutents that allow the redox potential of the ETM to be modified, such that the ETMS used in the methods of the invention are electrochemically distinguished from one another.

In a preferred embodiment, the ETMs are ferrocene derivatives that exhibit unique reversible redox potentials. Based on the oxidation and reduction potentials of the heterocyclic bases found in nucleic acids (Seidel, et al., J. Phys. Chem., 100: 4451 (1996); Steenken, et al., J. Am. Chem. Soc., 114: 4701, (1992); Steenken & Jovanovic, J.Am. Chem. Soc., 119: 617, (1997), the redox potentials of the ferrocene derivatives should range 0 to 520 mV.

As will be understood by those in the art, all of the ferrocene derivatives depicted herein may have additional atoms or structures, i.e., the ferrocene derivative of Structure 1 may be attached to nucleic acids, etc. Unless otherwise noted, the ferrocene derivatives depicted herein are attached to these additional structures via Y. For example, if the ferrocene derivative is to be attached to a nucleic acid (i.e., nucleosides, nucleic acid analogs), or other moiety such as a phosphoramidite, Y is attached to the either directly or through the use of a linker (L) as shown in structure 1. In addition, the ferrocene derivatives of the present invention may be substituted with one or more substitution groups, generally depicted herein as R. Both the R groups and the linker may be used to tune the redox potential of the ferrocene derivative.

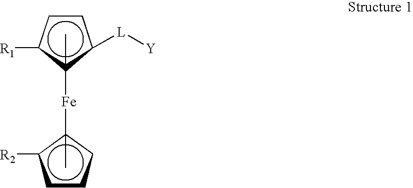

Suitable R groups include, but are not limited to, hydrogen, alkyl, alcohol, aromatic, amino, amido, nitro, ethers, esters, aldehydes, sulfonyl, silicon moieties, halogens, sulfur containing moieties, phosphorus containing moieties, and ethylene glycols. In the structures depicted herein, R is hydrogen when the position is unsubstituted. It should be noted that some positions may allow two substitution groups, R and R′, in which case the R and R′ groups may be either the same or different.

By “alkyl group” or grammatical equivalents herein is meant a straight or branched chain alkyl group, with straight chain alkyl groups being preferred. If branched, it may be branched at one or more positions, and unless specified, at any position. The alkyl group may range from about 1 to about 30 carbon atoms (C₁-C₃₀), with a preferred embodiment utilizing from about 1 to about 20 carbon atoms (C₁-C₂₀), with about C1 through about C12 to about C15 being preferred, and C1 to C5 being particularly preferred, although in some embodiments the alkyl group may be much larger. Also included within the definition of an alkyl group are cycloalkyl groups such as C5 and C6 rings, and heterocyclic rings with nitrogen, oxygen, sulfur or phosphorus. Alkyl also includes heteroalkyl, with heteroatoms of sulfur, oxygen, nitrogen, and silicone being preferred. Alkyl includes substituted alkyl groups. By “substituted alkyl group” herein is meant an alkyl group further comprising one or more substitution moieties “R”, as defined above.

By “amino groups” or grammatical equivalents herein is meant —NH₂, —NHR and —NR₂ groups, with R being as defined herein.

By “nitro group” herein is meant an —NO₂ group.

By “sulfur containing moieties” herein is meant compounds containing sulfur atoms, including but not limited to, thia-, thio- and sulfo- compounds, thiols (—SH and —SR), and sulfides (—RSR—). By “phosphorus containing moieties” herein is meant compounds containing phosphorus, including, but not limited to, phosphines and phosphates. By “silicon containing moieties” herein is meant compounds containing silicon.

By “ether” herein is meant an —O—R group. Preferred ethers include alkoxy groups, with —O—(CH₂)₂CH₃ and —O—(CH₂)₄CH₃ being preferred.

By “ester” herein is meant a —COOR group.

By “halogen” herein is meant bromine, iodine, chlorine, or fluorine. Preferred substituted alkyls are partially or fully halogenated alkyls such as CF₃, etc.

By “aldehyde” herein is meant —RCHO groups.

By “alcohol” herein is meant —OH groups, and alkyl alcohols —ROH.

By “amido” herein is meant —RCONH— or RCONR— groups.

By “ethylene glycol” or “(poly)ethylene glycol” herein is meant a —(O—CH₂—CH₂)ₙ— group, although each carbon atom of the ethylene group may also be singly or doubly substituted, i.e. —(O—CR₂—CR₂)ₙ—, with R as described above. Ethylene glycol derivatives with other heteroatoms in place of oxygen (i.e. —(N—CH₂—CH₂)ₙ— or —(S—CH₂—CH₂)ₙ—, or with substitution groups) are also preferred.

Preferred substitution groups include, but are not limited to, methyl, ethyl, propyl, alkoxy groups such as —O—(CH₂)₂CH₃ and —O—(CH₂)₄CH₃ and ethylene glycol and derivatives thereof.

In a preferred embodiment, Y is attached to a nucleic acid or other moiety through the use of a linker (L). Preferably, L is a short linker of about 1 to about 10 atoms, with from 1 to 5 atoms being preferred, that may or may not contain alkene, alkynyl, amine, amide, azo, imine, oxo, etc., bonds. Linkers are known in the art; for example, homo-or hetero-bifunctional linkers as are well known (see 1994 Pierce Chemical Company catalog, technical section on cross-linkers, pages 155-200, incorporated herein by reference). Preferred L linkers include, but are not limited to, alkoxy groups (including mono-alkoxy groups and dialkoxy groups), with short alkyl groups being preferred, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C₂ alkene being especially preferred. Z may also be a sulfone group, forming sulfonamide linkages.

Particularly preferred ferrocene derivatives of this embodiment are depicted in the Figures. For example, preferred ferrocene derivatives include, but are not limited to: CT170, N230, SJ9, SJ63, K161, N₂₀₄, SJ42, N221,CT171, CT186, and SJ21 (see Figures for chemical structures of the compounds listed).

In a preferred embodiment, the ETMs are ferrocene phosphoramidites derivatives that exhibit unique redox potentials. Preferred structures for ferrocene phosphoramidites derivatives are shown in the Figures and include structures K161 and N_{204.}

In a preferred embodiment, the ETMs are ferrocene labeled dideoxynucleotide triposhates as shown in FIG. 20. In the embodiments shown in FIG. 20, the ETM can be attached off of the ribose ring or off of the base.

In a preferred embodiment, the ETMs are polypyridine Ru²⁺ derivatives that exhibit unique reversible redox potentials. Based on the oxidation and reduction potentials of the heterocyclic bases found in nucleic acids (Seidel, et al., J. Phys. Chem., 100: 4451 (1996); Steenken, et al., J. Am. Chem. Soc., 114: 4701, (1992); Steenken & Jovanovic, J.Am. Chem. Soc., 119: 617, (1997), the redox potentials of the polypyridine Ru²⁺ derivatives should range −600 mV to 600 mV.

In a preferred embodiment, the high oxidation potential for [bpy)₃Ru]²⁺ and [(phen)₃Ru]²⁺ (i.e., 1.25 V,) is reduced by replacing one of the polypyridine ligands with a negatively charged ligand (e.g., hydroxamate, acetoacetate) (see Figures). provide the coordination atoms for the binding of the metal ion. As will be appreciated by those in the art, the number and nature of the co-ligands will depend on the coordination number of the metal ion. Mono-, di- or polydentate co-ligands may be used at any position.

Additional fine-tuning of the redox potential can be achieved through the selection of coordinated ligands. Substitution of hydroxamic acid can be combined with substituted bipyridines to design new complexes with “tuned” redox potentials.

Alternative ligands, such as acetylacetonato may also be used to tune the redox potential of polypyridine Ru²⁺ derivatives.

Other examples of suitable ligands include, but are not limited to, ligands that fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lₘ). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam), EDTA, EGTA and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp73-98), 21.1 (pp. 813-898) and 21.3 (pp 915-957), all of which are hereby expressly incorporated by reference.

Suitable sigma donating ligands using carbon, oxygen, sulfur and phosphorus are known in the art. For example, suitable sigma carbon donors are found in Cotton and Wilkenson, Advanced Organic Chemistry, 5th Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson.

The oxygen, sulfur, phosphorus and nitrogen-donating ligands are attached in such a manner as to allow the heteroatoms to serve as coordination atoms.

In a preferred embodiment, organometallic ligands are used. In addition to purely organic compounds for use as redox moieties, and various transition metal coordination complexes with δ-bonded organic ligand with donor atoms as heterocyclic or exocyclic substituents, there is available a wide variety of transition metal organometallic compounds with n-bonded organic ligands (see Advanced Inorganic Chemistry, 5th Ed., Cotton & Wilkinson, John Wiley & Sons, 1988, chapter 26; Organometallics, A Concise Introduction, Elschenbroich et al., 2nd Ed., 1992, VCH; and Comprehensive Organometallic Chemistry II, A Review of the Literature 1982-1994, Abel et al. Ed., Vol. 7, chapters 7, 8, 10 & 11, Pergamon Press, hereby expressly incorporated by reference). Such organometallic ligands include cyclic aromatic compounds such as the cyclopentadienide ion [C₅H₅(−1)] and various ring substituted and ring fused derivatives, such as the indenylide (−1) ion, that yield a class of bis(cyclopentadieyl) metal compounds, (i.e. the metallocenes); see for example Robins et al., J. Am. Chem. Soc. 104:1882-1893 (1982); and Gassman et al., J. Am. Chem. Soc. 108:42284229 (1986), incorporated by reference. Of these, ferrocene [(C₅H₅)₂Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to either the ribose ring or the nucleoside base of nucleic acid. Other potentially suitable organometallic ligands include cyclic arenes such as benzene, to yield bis(arene)metal compounds and their ring substituted and ring fused derivatives, of which bis(benzene)chromium is a prototypical example, Other acyclic π-bonded ligands such as the allyl(−1) ion, or butadiene yield potentially suitable organometallic compounds, and all such ligands, in conjuction with other n-bonded and δ-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene are derivatized.

As described herein, any combination of ligands may be used. Preferred combinations include: a) all ligands are nitrogen donating ligands; b) all ligands are organometallic ligands; and c) the ligand at the terminus of the conductive oligomer is a metallocene ligand and the ligand provided by the nucleic acid is a nitrogen donating ligand, with the other ligands, if needed, are either nitrogen donating ligands or metallocene ligands, or a mixture.

In addition, other metal ions can be utilized such as Os²⁺ polypyridine complexes.

Preferred embodiments for four polypyridine Ru²⁺ derivatives that may be used in the Sanger sequencing methods described herein are shown in the Figures. Modification of the length of the linker used to conjugate the redox-active Ru²⁺ complex to the heterocyclic base can be used to optimize polymerase recognition and electrophoretic mobility.

Preferred structures for polypyridine Ru²⁺ phosphoramidites derivatives are shown in the Figures.

As will be appreciated by those of skill in the art, numerous methods may be used to make the ETMs of the present invention. Methods for preparing ferrocene derivatives with multiple redox potentials are shown in the Figures and described in the examples. Generally, groups that are substantially electron withdrawing can be used to increase the redox potential of the ferrocene moiety, while groups that are substantially electron donating can be used to decrease the redox potential.

In a preferred embodiment, the attachment of the nucleic acid and the ETM is done via attachment to the backbone of the nucleic acid. This may be done in a number of ways, including attachment to a ribose of the ribose-phosphate backbone, or to the phosphate of the backbone, or other groups of analogous backbones.

As a preliminary matter, it should be understood that the site of attachment in this embodiment may be to a 3′ or 5′ terminal nucleotide, or to an internal nucleotide, as is more fully described below.

In a preferred embodiment, the ETM is attached to the ribose of the ribose-phosphate backbone. This may be done in several ways. As is known in the art, nucleosides that are modified at either the 2′ or 3′ position of the ribose with amino groups, sulfur groups, silicone groups, phosphorus groups, or oxo groups can be made (Imazawa et al., J. Org. Chem., 44:2039 (1979); Hobbs et al., J. Org. Chem. 42(4):714 (1977); Verheyden et al., J. Orrg. Chem. 36(2):250 (1971); McGee et al., J. Org. Chem. 61:781-785 (1996); Mikhailopulo et al., Liebigs. Ann. Chem. 513-519 (1993); McGee et al., Nucleosides & Nucleotides 14(6):1329 (1995), all of which are incorporated by reference). These modified nucleosides are then used to add the ETMs.

A preferred embodiment utilizes amino-modified nucleosides. These amino-modified riboses can then be used to form either amide or amine linkages to the ETMs. In a preferred embodiment, the amino group is attached directly to the ribose, although as will be appreciated by those in the art, short linkers such as those described herein for “L” may be present between the amino group and the ribose.

In a preferred embodiment, an amide linkage is used for attachment to the ribose.

In a preferred embodiment, the ferrocene derivatives with multi-potentials are conjugated to nucleic acids using a post-synthesis methodology. In this embodiment, nucleosides are modified as described above with a reactive group, such as NH₂, OH, phosphate, etc. Preferably, the reactive group on the modified nucleoside reacts with an activated group, attached to the ferrocene via a linker to form a covalent bond, such that the modified nucleoside is attached to the ferrocene via a linker.

Preferred post synthesis methods are shown in the Examples and in the Figures.

Methods for preparing polypyridine Ru²⁺derivatives with multiple redox potentials are shown in the Figures and described in the examples. Generally, a modular approach is used for synthesizing the polypyridine Ru²⁺derivatives, as the various components can be modified and intercahnged. For example, the following components are utilized to synthesize the polypyridine Ru²⁺derivatives of the present invention: (a) bis-substituted Ru²⁺ precursors (R₂ bpy)₂RuCl₂; (b) sustituted hydroxamic acids bearing a functionalized linker such as those described herein; and (c) modified dideoxynucleosides (tides).

As will be appreciated by those of skill in the art, ETMs with unique redox potentials may also be used in genotyping reaction, particularly, for SNP detection. In genotyping embodiments, a plurality of capture probes are made each with at least one ETM with a unique redox potential. This is analogous to the “two color” or “four color” idea of competitive hybridization, and is also analogous to sequencing by hybridization. For example, sequencing by hybridization has been described (Drmanac et al., Genomics 4:114 (1989); Koster et al., Nature Biotechnology 14:1123 (1996); U.S. Pat. Nos. 5,525,464; 5,202,231 and 5,695,940, among others, all of which are hereby expressly incorporated by reference in their entirety).

In a preferred embodiment, probes with a plurality of ETMs are provided to allow more sensitive detection limits. Accordingly, pluralities of ETMS are preferred, with at least about 2 ETMs per probe being preferred, and at least about 10 being particularly preferred and at least about 20 to 50 being especially preferred, In some instances, vary large numbers of ETMs (100 to 1000) can be used.

In a preferred embodiment, the ETMS are ferrocenes. Thus, “multi-ferrocene probes” or “poly-ferrocene probes” are provided. As will be appreciated by those of skill in the art, the probes may be capture probes as described herein, or other probes, such as label probes, amplifier probes, label probes comprising recruitment linkers or signal carriers may be used in the invention. For a discussion of label probes, amplifier probes, etc., see U.S. Ser. No. 09/626,096, filed Jul. 27, 1999, hereby incorporated by reference in its entirety.

Other configurations for providing probes with a plurality of ETMs are disclosed in U.S. Ser. No. 09/626,096, filed Jul. 27, 1999, hereby incorporated by reference in its entirety.

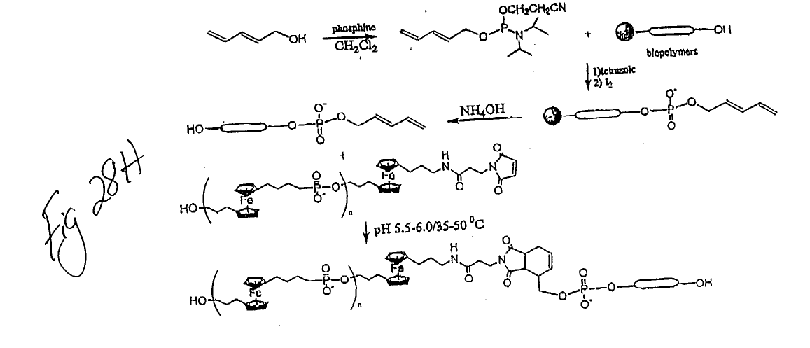

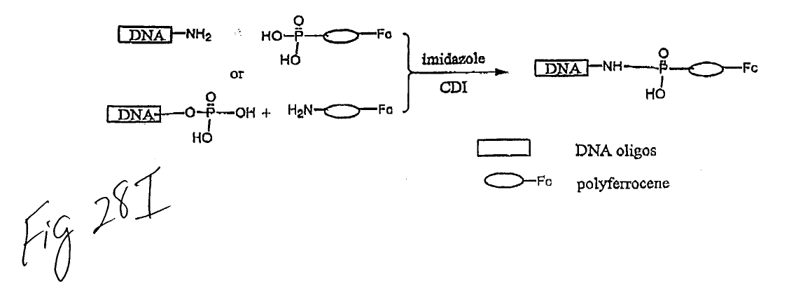

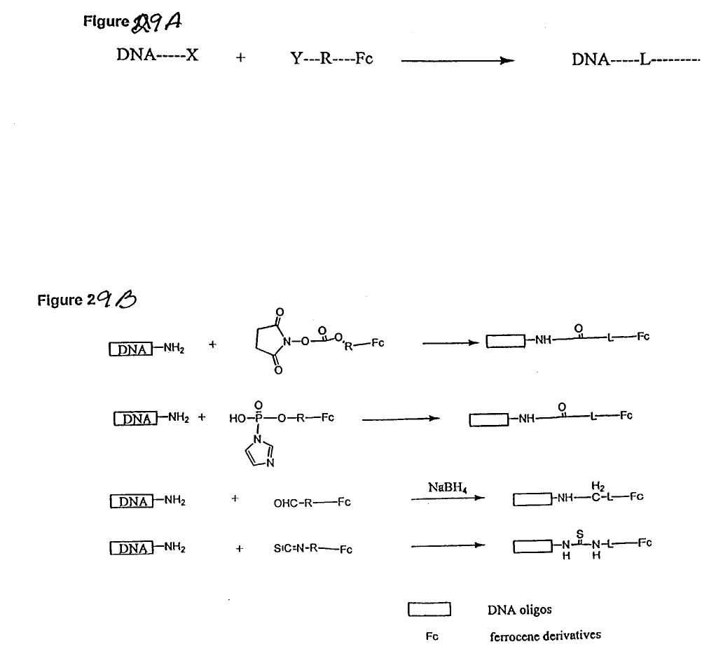

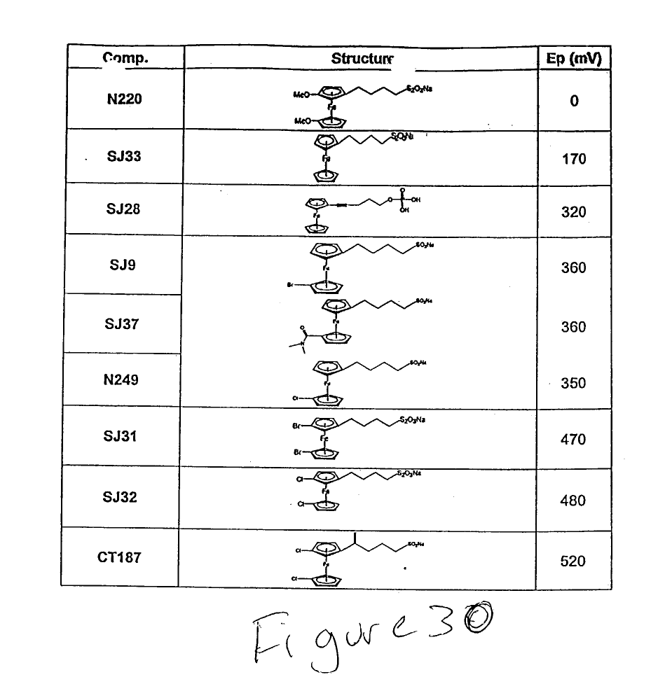

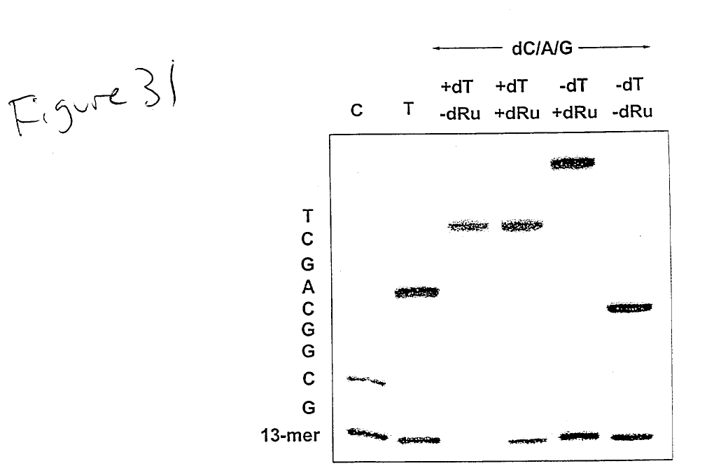

Preferably, water-soluble multi-or poly-ferrocene probes are made. Methods for preparing multi-ferrocene probes are shown in the FIGS. 28A-I.

In a preferred embodiment, single base extension (SBE; sometimes referred to as “minisequencing”) is used to determine the identity of the base at the detection position. Briefly, SBE is a technique that utilizes an extension primer that hybridizes to the target nucleic acid immediately adjacent to the detection position. A polymerase (generally a DNA polymerase) is used to extend the 3′ end of the primer with a nucleotide analog labeled with an ETM as described herein. A nucleotide is only incorporated into the growing nucleic acid strand if it is complementary to the base in the target strand at the detection position. The nucleotide is derivatized such that no further extensions can occur, so only a single nucleotide is added. Once the labeled nucleotide is added, detection of the ETM proceeds as outlined herein.

As will be appreciated by those in the art, the determination of the base at the detection position can proceed in several ways. In a preferred embodiment, the reaction is run with all four nucleotides, each with a different label, e.g. ETMs with different redox potentials, as is generally outlined herein. Alternatively, a single label is used, by using four electrode pads as outlined above or sequential reactions; for example, dATP can be added to the assay complex, and the generation of a signal evaluated; the dATP can be removed and dTTP added, etc.

The reaction is initiated by introducing the assay complex comprising the target sequence (i.e. the array) to a solution comprising a first nucleotide analog. By “nucleotide analog” in this context herein is meant a deoxynucleoside-triphosphate (also called deoxynucleotides or dNTPs, i.e. dATP, dTTP, dCTP and dGTP), that is further derivatized to be chain terminating. The nucleotides may be naturally occurring, such as deoxynucleotides, or non-naturally occuring. Preferred embodiments utilize dideoxy-triphosphate nucleotides (ddNTPs). Generally, a set of nucleotides comprising ddATP, ddCTP, ddGTP and ddTTP is used. In a preferred embodiment, each analog should be labeled with an ETM of different redox potential such that detecting the redox potential of the extended product is indicative of which label was incorporated.

In addition, as will be appreciated by those in the art, the single base extension reactions of the present invention allow the precise incorporation of modified bases into a growing nucleic acid strand. Thus, any number of modified nucleotides may be incorporated for any number of reasons, including probing structure-function relationships (e.g. DNA:DNA or DNA:protein interactions), cleaving the nucleic acid, crosslinking the nucleic acid, incorporate mismatches, etc.

In addition to a first nucleotide, the solution also comprises an extension enzyme, generally a DNA polymerase. Suitable DNA polymerases include, but are not limited to, the Klenow fragment of DNA polymerase 1, SEQUENASE 1.0 and SEQUENASE 2.0 (U.S. Biochemical), T5 DNA polymerase and Phi29 DNA polymerase. If the NTP is complementary to the base of the detection position of the target sequence, which is adjacent to the extension primer, the extension enzyme will add it to the extension primer at the interrogation position. Thus, the extension primer is modified, i.e. extended, to form a modified primer, sometimes referred to herein as a “newly synthesized strand”. If desired, the temperature of the reaction can be adjusted (or cycled) such that amplification occurs, generating a plurality of modified primers.

As will be appreciated by those in the art, the configuration of the SBE system can take on several forms, but generally result in the formation of assay complexes on a surfaces, frequently an electrode, as a result of hybridization of a target sequence (either the target sequence of the sample or a sequence generated in the assay) to a capture probe on the surface. As is more fully outlined herein, this may be direct or indirect (e.g. through the use of sandwich type systems) hybridization as described in U.S. Ser. No. 09/626,096, filed July26, 1999, incorporated herein by reference. Once the assay complexes are formed, the presence or absence of the ETMs are detected as is described below and in U.S. Pat. Nos. 5,591,578; 5,824,473; 5,770,369; 5,705,348 and 5,780,234; U.S. Ser. Nos. 08/911,589; 09/135,183; 09/306,653; 09/134,058; 09/295,691; 09/238,351; 09/245,105 and 09/338,726; and PCT applications WO98/20162; WO 00/16089; PCT US99/01705; PCT US99/01703; PCT US00/10903 and PCT US99/10104, all of which are expressly incorporated herein by reference in their entirety.

In general, there are two basic detection mechanisms. In a preferred embodiment, detection of an ETM is based on electron transfer through the stacked n-orbitals of double stranded nucleic acid. This basic mechanism is described in U.S. Pat. Nos. 5,591,578, 5,770,369, 5,705,348, and PCT US97/20014 and is termed “mechanism-1” herein. Briefly, previous work has shown that electron transfer can proceed rapidly through the stacked n-orbitals of double stranded nucleic acid, and significantly more slowly through single-stranded nucleic acid. Thus, by adding ETMs (either covalently to one of the strands or non-covalently to the hybridization complex through the use of hybridization indicators, described below) to a nucleic acid that is attached to a detection electrode via a conductive oligomer, electron transfer between the ETM and the electrode, through the nucleic acid and conductive oligomer, may be detected.

Alternatively, the ETM can be detected, not necessarily via electron transfer through nucleic acid, but rather can be directly detected on an electrode comprising a self-assembled monolayer (SAM); that is, the electrons from the ETMs need not travel through the stacked n orbitals in order to generate a signal. As above, in this embodiment, the detection electrode preferably comprises a self-assembled monolayer (SAM) that serves to shield the electrode from redox-active species in the sample. In this embodiment, the presence of ETMs on the surface of a SAM, that has been formulated to comprise slight “defects” (sometimes referred to herein as “microconduits”, “nanoconduits” or “electroconduits”) can be directly detected. This basic idea is termed “mechanism-2” herein. Essentially, the electroconduits allow particular ETMs access to the surface. Without being bound by theory, it should be noted that the configuration of the electroconduit depends in part on the ETM chosen. For example, the use of relatively hydrophobic ETMs allows the use of hydrophobic electroconduit forming species, which effectively exclude hydrophilic or charged ETMs. Similarly, the use of more hydrophilic or charged species in the SAM may serve to exclude hydrophobic ETMs. Compositions, methods of making and using SAMS for use in genotyping assays are described in U.S. Ser. No. 09/626,096, filed Jul. 26, 1999, incorporated herein by reference.

The above system finds particular utility in array formats, i.e. wherein there is a matrix of addressable detection electrodes (herein generally referred to “pads”, “addresses” or “micro-locations”). See U.S. Ser. No. 09/626,096, filed Jul. 26, 1999, incorporated herein by reference.

For a discussion of hybridization conditions, reaction conditions, methods of detecting target sequences using probes comprising ETMs on solid substrates see U.S. Ser. No. 09/626,096, filed Jul. 27, 1999, hereby incorporated by reference in its entirety.

Once the assay complexes of the hybridized SBE products are made, detection proceeds with electronic initiation. By “assay complexes” herein is meant the population of sequencing probes generated from the Sanger sequencing reactions or the hybridization complexes generated from SBE genotyping reactions. Without being limited by the mechanism or theory, detection is based on the trransfer of electrons from the ETM to the electrode.

Detection of electron transfer, i.e. the presence of the ETMs, is generally initiated electronically, with voltage being preferred. A potential is applied to the assay complex. Precise control and variations in the applied potential can be via a potentiostat and either a three electrode system (one reference, one sample (or working) and one counter electrode) or a two electrode system (one sample and one counter electrode). This allows matching of applied potential to peak potential of the system which depends in part on the choice of ETMs and in part on the conductive oligomer used, the composition and integrity of the monolayer, and what type of reference electrode is used. As described herein, ferrocene is a preferred ETM.

In a preferred embodiment, a co-reductant or co-oxidant (collectively, co-redoxant) is used, as an additional electron source or sink. See generally Sato et al., Bull. Chem. Soc. Jpn 66:1032 (1993); Uosaki et al., Electrochimica Acta 36:1799 (1991); and Alleman et al., J. Phys. Chem 100:17050 (1996); all of which are incorporated by reference.

In a preferred embodiment, an input electron source in solution is used in the initiation of electron transfer, preferably when initiation and detection are being done using DC current or at AC frequencies where diffusion is not limiting. In general, as will be appreciated by those in the art, preferred embodiments utilize monolayers that contain a minimum of “holes”, such that short-circuiting of the system is avoided. This may be done in several general ways. In a preferred embodiment, an input electron source is used that has a lower or similar redox potential than the ETM of the label probe.

Thus, at voltages above the redox potential of the input electron source, both the ETM and the input electron source are oxidized and can thus donate electrons; the ETM donates an electron to the electrode and the input source donates to the ETM. For example, ferrocene, as a ETM attached to the compositions of the invention as described in the examples, has a redox potential of roughly 200 mV in aqueous solution (which can change significantly depending on what the ferrocene is bound to, the manner of the linkage and the presence of any substitution groups). Ferrocyanide, an electron source, has a redox potential of roughly 200 mV as well (in aqueous solution). Accordingly, at or above voltages of roughly 200 mV, ferrocene is converted to ferricenium, which then transfers an electron to the electrode. Now the ferricyanide can be oxidized to transfer an electron to the ETM. In this way, the electron source (or co-reductant) serves to amplify the signal generated in the system, as the electron source molecules rapidly and repeatedly donate electrons to the ETM attached to the nucleic acid. The rate of electron donation or acceptance will be limited by the rate of diffusion of the co-reductant, the electron transfer between the co-reductant and the ETM, which in turn is affected by the concentration and size, etc.

Alternatively, input electron sources that have lower redox potentials than the ETM are used. At voltages less than the redox potential of the ETM, but higher than the redox potential of the electron source, the input source such as ferrocyanide is unable to be oxided and thus is unable to donate an electron to the ETM; i.e. no electron transfer occurs. Once ferrocene is oxidized, then there is a pathway for electron transfer.

In an alternate preferred embodiment, an input electron source is used that has a higher redox potential than the ETM of the label probe. For example, luminol, an electron source, has a redox potential of roughly 720 mV. At voltages higher than the redox potential of the ETM, but lower than the redox potential of the electron source, i.e. 200-720 mV, the ferrocene is oxided, and transfers a single electron to the electrode via the conductive oligomer. However, the ETM is unable to accept any electrons from the luminol electron source, since the voltages are less than the redox potential of the luminol. However, at or above the redox potential of luminol, the luminol then transfers an electron to the ETM, allowing rapid and repeated electron transfer. In this way, the electron source (or co-reductant) serves to amplify the signal generated in the system, as the electron source molecules rapidly and repeatedly donate electrons to the ETM of the label probe.

Luminol has the added benefit of becoming a chemiluminiscent species upon oxidation (see Jirka et al., Analytica Chimica Acta 284:345 (1993)), thus allowing photo-detection of electron transfer from the ETM to the electrode. Thus, as long as the luminol is unable to contact the electrode directly, i.e. in the presence of the SAM such that there is no efficient electron transfer pathway to the electrode, luminol can only be oxidized by transferring an electron to the ETM on the label probe. When the ETM is not present, i.e. when the target sequence is not hybridized to the composition of the invention, luminol is not significantly oxidized, resulting in a low photon emission and thus a low (if any) signal from the luminol. In the presence of the target, a much larger signal is generated. Thus, the measure of luminol oxidation by photon emission is an indirect measurement of the ability of the ETM to donate electrons to the electrode. Furthermore, since photon detection is generally more sensitive than electronic detection, the sensitivity of the system may be increased. Initial results suggest that luminescence may depend on hydrogen peroxide concentration, pH, and luminol concentration, the latter of which appears to be non-linear.

Suitable electron source molecules are well known in the art, and include, but are not limited to, ferricyanide, and luminol.

Alternatively, output electron acceptors or sinks could be used, i.e. the above reactions could be run in reverse, with the ETM such as a metallocene receiving an electron from the electrode, converting it to the metallicenium, with the output electron acceptor then accepting the electron rapidly and repeatedly. In this embodiment, cobalticenium is the preferred ETM.

The presence of the ETMs at the surface of the monolayer can be detected in a variety of ways. A variety of detection methods may be used, including, but not limited to, optical detection (as a result of spectral changes upon changes in redox states), which includes fluorescence, phosphorescence, luminiscence, chemiluminescence, electrochemiluminescence, and refractive index; and electronic detection, including, but not limited to, amperommetry, voltammetry, capacitance and impedence. These methods include time or frequency dependent methods based on AC or DC currents, pulsed methods, lock-in techniques, filtering (high pass, low pass, band pass), and time-resolved techniques including time-resolved fluorescence.

In one embodiment, the efficient transfer of electrons from the ETM to the electrode results in stereotyped changes in the redox state of the ETM. With many ETMs including the complexes of ruthenium containing bipyridine, pyridine and imidazole rings, these changes in redox state are associated with changes in spectral properties. Significant differences in absorbance are observed between reduced and oxidized states for these molecules. See for example Fabbrizzi et al., Chem. Soc. Rev. 1995 ppl97-202). These differences can be monitored using a spectrophotometer or simple photomultiplier tube device.

In this embodiment, possible electron donors and acceptors include all the derivatives listed above for photoactivation or initiation. Preferred electron donors and acceptors have characteristically large spectral changes upon oxidation and reduction resulting in highly sensitive monitoring of electron transfer. Such examples include Ru(NH₃)₄py and Ru(bpy)₂im as preferred examples. It should be understood that only the donor or acceptor that is being monitored by absorbance need have ideal spectral characteristics.

In a preferred embodiment, the electron transfer is detected fluorometrically. Numerous transition metal complexes, including those of ruthenium, have distinct fluorescence properties. Therefore, the change in redox state of the electron donors and electron acceptors attached to the nucleic acid can be monitored very sensitively using fluorescence, for example with Ru(4,7-biphenyl₂-phenanthroline)₃²⁺. The production of this compound can be easily measured using standard fluorescence assay techniques. For example, laser induced fluorescence can be recorded in a standard single cell fluorimeter, a flow through “on-line” fluorimeter (such as those attached to a chromatography system) or a multi-sample “plate-reader” similar to those marketed for 96-well immuno assays.

Alternatively, fluorescence can be measured using fiber optic sensors with nucleic acid probes in solution or attached to the fiber optic. Fluorescence is monitored using a photomultiplier tube or other light detection instrument attached to the fiber optic. The advantage of this system is the extremely small volumes of sample that can be assayed.

In addition, scanning fluorescence detectors such as the Fluorimager sold by Molecular Dynamics are ideally suited to monitoring the fluorescence of modified nucleic acid molecules arrayed on solid surfaces. The advantage of this system is the large number of electron transfer probes that can be scanned at once using chips covered with thousands of distinct nucleic acid probes.

Many transition metal complexes display fluorescence with large Stokes shifts. Suitable examples include bis- and trisphenanthroline complexes and bis- and trisbipyridyl complexes of transition metals such as ruthenium (see Juris, A., Balzani, V., et. al. Coord. Chem. Rev., V. 84, p. 85-277, 1988). Preferred examples display efficient fluorescence (reasonably high quantum yields) as well as low reorganization energies. These include Ru(4,7-biphenyl₂-phenanthroline)₃²⁺, Ru(4,4′-diphenyl-2,2′-bipyridine)₃²⁺ and platinum complexes (see Cummings et al., J. Am. Chem. Soc. 118:1949-1960 (1996), incorporated by reference). Alternatively, a reduction in fluorescence associated with hybridization can be measured using these systems.

In a further embodiment, electrochemiluminescence is used as the basis of the electron transfer detection. With some ETMs such as Ru²⁺(bpy)₃, direct luminescence accompanies excited state decay. Changes in this property are associated with nucleic acid hybridization and can be monitored with a simple photomultiplier tube arrangement (see Blackburn, G. F. Clin. Chem. 37: 1534-1539 (1991); and Juris et al., supra.

In a preferred embodiment, electronic detection is used, including amperommetry, voltammetry, capacitance, and impedance. Suitable techniques include, but are not limited to, electrogravimetry; coulometry (including controlled potential coulometry and constant current coulometry); voltametry (cyclic voltametry, pulse voltametry (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry; and photoelectrochemistry.

In a preferred embodiment, monitoring electron transfer is via amperometric detection. This method of detection involves applying a potential (as compared to a separate reference electrode) between the nucleic acid-conjugated electrode and a reference (counter) electrode in the sample containing target genes of interest. Electron transfer of differing efficiencies is induced in samples in the presence or absence of target nucleic acid; that is, the presence or absence of the target nucleic acid, and thus the label probe, can result in different currents.

The device for measuring electron transfer amperometrically involves sensitive current detection and includes a means of controlling the voltage potential, usually a potentiostat. This voltage is optimized with reference to the potential of the electron donating complex on the label probe. Possible electron donating complexes include those previously mentioned with complexes of iron, osmium, platinum, cobalt, rhenium and ruthenium being preferred and complexes of iron being most preferred.

In a preferred embodiment, alternative electron detection modes are utilized. For example, potentiometric (or voltammetric) measurements involve non-faradaic (no net current flow) processes and are utilized traditionally in pH and other ion detectors. Similar sensors are used to monitor electron transfer between the ETM and the electrode. In addition, other properties of insulators (such as resistance) and of conductors (such as conductivity, impedance and capacitance) could be used to monitor electron transfer between ETM and the electrode. Finally, any system that generates a current (such as electron transfer) also generates a small magnetic field, which may be monitored in some embodiments.

It should be understood that one benefit of the fast rates of electron transfer observed in the compositions of the invention is that time resolution can greatly enhance the signal-to-noise results of monitors based on absorbance, fluorescence and electronic current. The fast rates of electron transfer of the present invention result both in high signals and stereotyped delays between electron transfer initiation and completion. By amplifying signals of particular delays, such as through the use of pulsed initiation of electron transfer and “lock-in” amplifiers of detection, and Fourier transforms.

In a preferred embodiment, electron transfer is initiated using alternating current (AC) methods. Without being bound by theory, it appears that ETMs, bound to an electrode, generally respond similarly to an AC voltage across a circuit containing resistors and capacitors. Basically, any methods which enable the determination of the nature of these complexes, which act as a resistor and capacitor, can be used as the basis of detection. Surprisingly, traditional electrochemical theory, such as exemplified in Laviron et al., J. Electroanal. Chem. 97:135 (1979) and Laviron et al., J. Electroanal. Chem. 105:35 (1979), both of which are incorporated by reference, do not accurately model the systems described herein, except for very small E_{AC} (less than 10 mV) and relatively large numbers of molecules. That is, the AC current (I) is not accurately described by Laviron's equation. This may be due in part to the fact that this theory assumes an unlimited source and sink of electrons, which is not true in the present systems.

The AC voltametry theory that models these systems well is outlined in O'Connor et al., J. Electroanal. Chem. 466(2):197-202 (1999), hereby expressly incorporated by reference. The equation that predicts these systems is shown below as Equation 1:

$${i}_{avg} = 2 {nfFN}_{total} \cdot \frac{sinh [ \frac{n F}{RT} \cdot {E}_{A C} ]}{cosh [ \frac{n F}{RT} \cdot {E}_{A C} ] + cosh [ \frac{n F}{RT} ( {E}_{D C} - {E}_{O} ) ]} \qquad Equation 1$$

In Equation 1, n is the number of electrons oxidized or reduced per redox molecule, f is the applied frequency, F is Faraday's constant, Nₜₒₜₐₗ is the total number of redox molecules, E_{O} is the formal potential of the redox molecule, R is the gas constant, T is the temperature in degrees Kelvin, and E_{DC} is the electrode potential. The model fits the experimental data very well. In some cases the current is smaller than predicted, however this has been shown to be caused by ferrocene degradation which may be remedied in a number of ways.

In addition, the faradaic current can also be expressed as a function of time, as shown in Equation 2:

$${I}_{f} ( t ) = \frac{{q}_{e} {N}_{total} n F}{2 RT ( cosh [ \frac{n F}{RT} ( V ( t ) - {E}_{0} ) ] + 1 )} \cdot \frac{ⅆ V ( t )}{ⅆ t} \qquad Equation 2$$

I_{F} is the Faradaic current and qₑ is the elementary charge.

However, Equation 1 does not incorporate the effect of electron transfer rate nor of instrument factors. Electron transfer rate is important when the rate is close to or lower than the applied frequency. Thus, the true i_{AC} should be a function of all three, as depicted in Equation 3.

### Equation 3

i_{AC}=f(Nernst factors)f(k_{ET})f(instrument factors)

These equations can be used to model and predict the expected AC currents in systems which use input signals comprising both AC and DC components. As outlined above, traditional theory surprisingly does not model these systems at all, except for very low voltages.

In general, non-specifically bound label probes/ETMs show differences in impedance (i.e. higher impedances) than when the label probes containing the ETMs are specifically bound in the correct orientation. In a preferred embodiment, the non-specifically bound material is washed away, resulting in an effective impedance of infinity. Thus, AC detection gives several advantages as is generally discussed below, including an increase in sensitivity, and the ability to “filter out” background noise. In particular, changes in impedance (including, for example, bulk impedance) as between non-specific binding of ETM-containing probes and target-specific assay complex formation may be monitored.

Accordingly, when using AC initiation and detection methods, the frequency response of the system changes as a result of the presence of the ETM. By “frequency response” herein is meant a modification of signals as a result of electron transfer between the electrode and the ETM. This modification is different depending on signal frequency. A frequency response includes AC currents at one or more frequencies, phase shifts, DC offset voltages, faradaic impedance, etc.

Once the assay complex including the target sequence and label probe is made, a first input electrical signal is then applied to the system, preferably via at least the sample electrode (containing the complexes of the invention) and the counter electrode, to initiate electron transfer between the electrode and the ETM. Three electrode systems may also be used, with the voltage applied to the reference and working electrodes. The first input signal comprises at least an AC component. The AC component may be of variable amplitude and frequency. Generally, for use in the present methods, the AC amplitude ranges from about 1 mV to about 1.1 V, with from about 10 mV to about 800 mV being preferred, and from about 10 mV to about 500 mV being especially preferred. The AC frequency ranges from about 0.01 Hz to about 100 MHz, with from about 10 Hz to about 10 MHz being preferred, and from about 100 Hz to about 20 MHz being especially preferred.

The use of combinations of AC and DC signals gives a variety of advantages, including surprising sensitivity and signal maximization.

In a preferred embodiment, the first input signal comprises a DC component and an AC component. That is, a DC offset voltage between the sample and counter electrodes is swept through the electrochemical potential of the ETM (for example, when ferrocene is used, the sweep is generally from 0 to 500 mV) (or alternatively, the working electrode is grounded and the reference electrode is swept from 0 to −500 mV). The sweep is used to identify the DC voltage at which the maximum response of the system is seen. This is generally at or about the electrochemical potential of the ETM. Once this voltage is determined, either a sweep or one or more uniform DC offset voltages may be used. DC offset voltages of from about −1 V to about +1.1 V are preferred, with from about −500 mV to about +800 mV being especially preferred, and from about −300 mV to about 500 mV being particularly preferred. In a preferred embodiment, the DC offset voltage is not zero. On top of the DC offset voltage, an AC signal component of variable amplitude and frequency is applied. If the ETM is present, and can respond to the AC perturbation, an AC current will be produced due to electron transfer between the electrode and the ETM.

For defined systems, it may be sufficient to apply a single input signal to differentiate between the presence and absence of the ETM (i.e. the presence of the target sequence) nucleic acid. Alternatively, a plurality of input signals are applied. As outlined herein, this may take a variety of forms, including using multiple frequencies, multiple DC offset voltages, or multiple AC amplitudes, or combinations of any or all of these.

Thus, in a preferred embodiment, multiple DC offset voltages are used, although as outlined above, DC voltage sweeps are preferred. This may be done at a single frequency, or at two or more frequencies.

In a preferred embodiment, the AC amplitude is varied. Without being bound by theory, it appears that increasing the amplitude increases the driving force. Thus, higher amplitudes, which result in higher overpotentials give faster rates of electron transfer. Thus, generally, the same system gives an improved response (i.e. higher output signals) at any single frequency through the use of higher overpotentials at that frequency. Thus, the amplitude may be increased at high frequencies to increase the rate of electron transfer through the system, resulting in greater sensitivity. In addition, this may be used, for example, to induce responses in slower systems such as those that do not possess optimal spacing configurations.

In a preferred embodiment, measurements of the system are taken at at least two separate amplitudes or overpotentials, with measurements at a plurality of amplitudes being preferred. As noted above, changes in response as a result of changes in amplitude may form the basis of identification, calibration and quantification of the system. In addition, one or more AC frequencies can be used as well.

In a preferred embodiment, the AC frequency is varied. At different frequencies, different molecules respond in different ways. As will be appreciated by those in the art, increasing the frequency generally increases the output current. However, when the frequency is greater than the rate at which electrons may travel between the electrode and the ETM, higher frequencies result in a loss or decrease of output signal. At some point, the frequency will be greater than the rate of electron transfer between the ETM and the electrode, and then the output signal will also drop.

In one embodiment, detection utilizes a single measurement of output signal at a single frequency. That is, the frequency response of the system in the absence of target sequence, and thus the absence of label probe containing ETMs, can be previously determined to be very low at a particular high frequency. Using this information, any response at a particular frequency, will show the presence of the assay complex. That is, any response at a particular frequency is characteristic of the assay complex. Thus, it may only be necessary to use a single input high frequency, and any changes in frequency response is an indication that the ETM is present, and thus that the target sequence is present.

In addition, the use of AC techniques allows the significant reduction of background signals at any single frequency due to entities other than the ETMs, i.e. “locking out” or “filtering” unwanted signals. That is, the frequency response of a charge carrier or redox active molecule in solution will be limited by its diffusion coefficient and charge transfer coefficient. Accordingly, at high frequencies, a charge carrier may not diffuse rapidly enough to transfer its charge to the electrode, and/or the charge transfer kinetics may not be fast enough. This is particularly significant in embodiments that do not have good monolayers, i.e. have partial or insufficient monolayers, i.e. where the solvent is accessible to the electrode. As outlined above, in DC techniques, the presence of “holes” where the electrode is accessible to the solvent can result in solvent charge carriers “short circuiting” the system, i.e. the reach the electrode and generate background signal. However, using the present AC techniques, one or more frequencies can be chosen that prevent a frequency response of one or more charge carriers in solution, whether or not a monolayer is present. This is particularly significant since many biological fluids such as blood contain significant amounts of redox active molecules which can interfere with amperometric detection methods.

In a preferred embodiment, measurements of the system are taken at at least two separate frequencies, with measurements at a plurality of frequencies being preferred. A plurality of frequencies includes a scan. For example, measuring the output signal, e.g., the AC current, at a low input frequency such as 1-20 Hz, and comparing the response to the output signal at high frequency such as 10-100 kHz will show a frequency response difference between the presence and absence of the ETM. In a preferred embodiment, the frequency response is determined at at least two, preferably at least about five, and more preferably at least about ten frequencies.

After transmitting the input signal to initiate electron transfer, an output signal is received or detected. The presence and magnitude of the output signal will depend on a number of factors, including the overpotential/amplitude of the input signal; the frequency of the input AC signal; the composition of the intervening medium; the DC offset; the environment of the system; the nature of the ETM; the solvent; and the type and concentration of salt. At a given input signal, the presence and magnitude of the output signal will depend in general on the presence or absence of the ETM, the placement and distance of the ETM from the surface of the monolayer and the character of the input signal. In some embodiments, it may be possible to distinguish between non-specific binding of label probes and the formation of target specific assay complexes containing label probes, on the basis of impedance.

In a preferred embodiment, the output signal comprises an AC current. As outlined above, the magnitude of the output current will depend on a number of parameters. By varying these parameters, the system may be optimized in a number of ways.

In general, AC currents generated in the present invention range from about 1 femptoamp to about 1 milliamp, with currents from about 50 femptoamps to about 100 microamps being preferred, and from about 1 picoamp to about 1 microamp being especially preferred.

In a preferred embodiment, the output signal is phase shifted in the AC component relative to the input signal. Without being bound by theory, it appears that the systems of the present invention may be sufficiently uniform to allow phase-shifting based detection. That is, the complex biomolecules of the invention through which electron transfer occurs react to the AC input in a homogeneous manner, similar to standard electronic components, such that a phase shift can be determined. This may serve as the basis of detection between the presence and absence of the ETM, and/or differences between the presence of target-specific assay complexes comprising label probes and non-specific binding of the label probes to the system components.

The output signal is characteristic of the presence of the ETM; that is, the output signal is characteristic of the presence of the target-specific assay complex comprising label probes and ETMs. In a preferred embodiment, the basis of the detection is a difference in the faradaic impedance of the system as a result of the formation of the assay complex. Faradaic impedance is the impedance of the system between the electrode and the ETM. Faradaic impedance is quite different from the bulk or dielectric impedance, which is the impedance of the bulk solution between the electrodes. Many factors may change the faradaic impedance which may not effect the bulk impedance, and vice versa. Thus, the assay complexes comprising the nucleic acids in this system have a certain faradaic impedance, that will depend on the distance between the ETM and the electrode, their electronic properties, and the composition of the intervening medium, among other things. Of importance in the methods of the invention is that the faradaic impedance between the ETM and the electrode is signficantly different depending on whether the label probes containing the ETMs are specifically or non-specifically bound to the electrode.

Accordingly, the present invention further provides apparatus for the detection of nucleic acids using AC detection methods. The apparatus includes a test chamber which has at least a first measuring or sample electrode, and a second measuring or counter electrode. Three electrode systems are also useful. The first and second measuring electrodes are in contact with a test sample receiving region, such that in the presence of a liquid test sample, the two electrodes may be in electrical contact.

In a preferred embodiment, the first measuring electrode comprises a single stranded nucleic acid capture probe covalently attached via an attachment linker, and a monolayer comprising conductive oligomers, such as are described herein.

The apparatus further comprises an AC voltage source electrically connected to the test chamber; that is, to the measuring electrodes. Preferably, the AC voltage source is capable of delivering DC offset voltage as well.

In a preferred embodiment, the apparatus further comprises a processor capable of comparing the input signal and the output signal. The processor is coupled to the electrodes and configured to receive an output signal, and thus detect the presence of the target nucleic acid.

Thus, the compositions of the present invention may be used in a variety of research, clinical, quality control, or field testing settings.

In a preferred embodiment, the probes are used in genetic diagnosis. For example, probes can be made using the techniques disclosed herein to detect target sequences such as the gene for nonpolyposis colon cancer, the BRCA1 breast cancer gene, P53, which is a gene associated with a variety of cancers, the Apo E4 gene that indicates a greater risk of Alzheimer's disease, allowing for easy presymptomatic screening of patients, mutations in the cystic fibrosis gene, or any of the others well known in the art.

In an additional embodiment, viral and bacterial detection is done using the complexes of the invention. In this embodiment, probes are designed to detect target sequences from a variety of bacteria and viruses. For example, current blood-screening techniques rely on the detection of anti-HIV antibodies. The methods disclosed herein allow for direct screening of clinical samples to detect HIV nucleic acid sequences, particularly highly conserved HIV sequences. In addition, this allows direct monitoring of circulating virus within a patient as an improved method of assessing the efficacy of anti-viral therapies. Similarly, viruses associated with leukemia, HTLV-I and HTLV-II, may be detected in this way. Bacterial infections such as tuberculosis, clymidia and other sexually transmitted diseases, may also be detected, for example using ribosomal RNA (rRNA) as the target sequences.

In a preferred embodiment, the nucleic acids of the invention find use as probes for toxic bacteria in the screening of water and food samples. For example, samples may be treated to lyse the bacteria to release its nucleic acid (particularly rRNA), and then probes designed to recognize bacterial strains, including, but not limited to, such pathogenic strains as, Salmonella, Campylobacter, Vibrio cholerae, Leishmania, enterotoxic strains of E. coli, and Legionnaire's disease bacteria. Similarly, bioremediation strategies may be evaluated using the compositions of the invention.

In a further embodiment, the probes are used for forensic “DNA fingerprinting” to match crime-scene DNA against samples taken from victims and suspects.

In an additional embodiment, the probes in an array are used for sequencing.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

### Derivation of Peak Finder Algorithm

The time dependent current I(t) generated by the detection system is processed by the lock-in amplifier. The component of time dependent current I(t) that has the same frequency as the fourth harmonic of the input voltage¹ is analyzed here², and expressed in terms of R(V) and phase e(V). They can be transformed into X(V) and Y(V) components by the following relations

X(V)=R(V)cos(θ−φ)

Y(V)=RV)sin(θ−φ) (5)

Where R is the magnitude of the current vector, φ is the phase shift as a function of V and φ is a reference phase. We have observed that shifting the phase can help to obtain a better signal in those files where the faradaic signal is mostly orthogonal to X or to Y. ¹The input voltage contains a dc ramp and an c sinusoid, described by the function Vᵢₙ (t)=Vₒ+rt+E_{ac}Sin(ωt) ²This method will serve as the brick to construct a robust algorithm for peak finding.

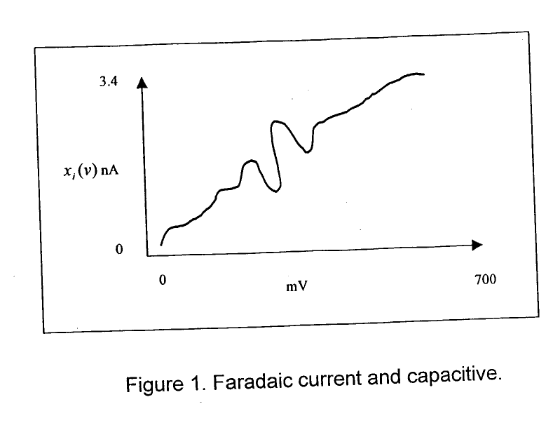

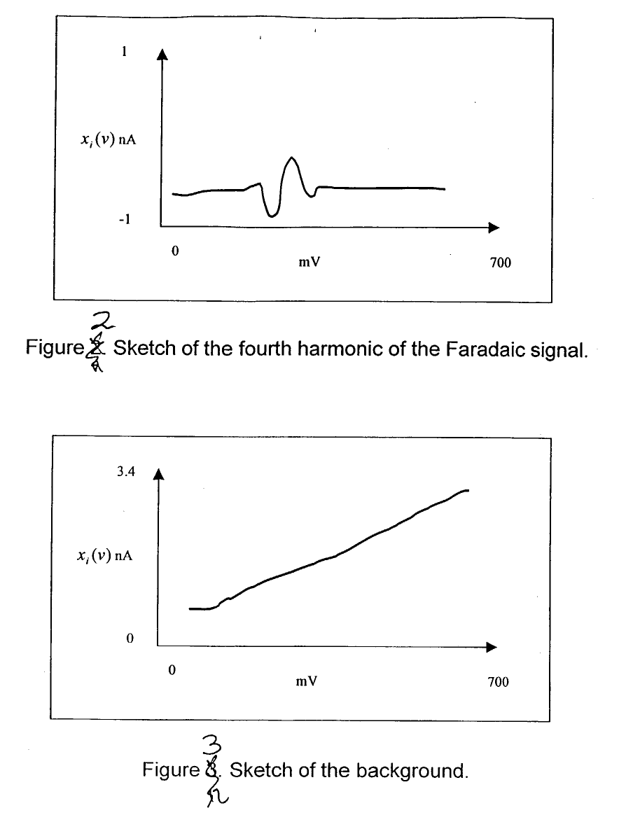

The sketch of a typical example of a clear X(V) component is represented in FIG. 1. It is modeled as a Faradaic signal superimposed on a capacitive background current. FIG. 2 sketches the signal component while FIG. 3 sketches the capacitive component.

The X(V) and Y(V) components of the current are assumed to be close to two fitting curves, each composed of the sum of two functions.

Fₓ(v)=F₁ₓ(v)+F₂ₓ(v)=G′″(Aₓₒ,Aₓ₁,Aₓ₂,v)+Aₓ₃+Aₓ₄v+Aₓ₅v²+A₆v³+A₇v⁴+Aₓ₈v⁵

F_{y}(v)=F_{1y}(v)+F_{2y}(v)=G′″(A_{yo},A_{y1},A_{y2},v)+A_{y3}+A_{y4}v+A_{y5}v²+A_{y6}v³+A_{y7}v⁴+A_{y8}v⁵ (6)

The fist part of the fitting curve (F₁ᵢ(V)) is the third derivative of a modified Gaussian distribution (FIG. 4). It simulates the fourth harmonic of the faradaic signal (FIG. 2). The second component, (F₂ᵢ(V)) a 5^{th} order polynomial³, is used to fit the background (FIG. 3). ³We initially used a 3^{rd} order polynomial, but a 5^{th} order approximates the background much better.

A good approximation to the fourth harmonic of the faradaic peak measured with a driving amplitude of E_{ac}=100 mV is given by the third derivative of a modified Gaussian distribution. The modified Gaussian distribution that we use relaxes the normalization condition by setting the integral equal to 1.

G(Aₒ,A₁,A₂,v)=AₒE^{-(v-A}₂)^{²}^{A}^{₁}^{²} (7)

The third derivative of Equation 7 is given by

G′″(Aₒ,A₁,A₂,v)=4AₒA₁⁴E^{-(v-A}^{₂}^{²}^{A}^{₁}^{²}(3−2A₁²(A₂−v)²)(v−A₂) (8)

The third derivative of the modified Gaussian (8) depends on three parameters, where Aₒ controls the amplitude of the signal. As seen in Equation (8), the amplitude of the curve also depends on A₁. A₁ is responsible for the width of the curve although it also plays a role in the amplitude. Equation (9) illustrates the effect of A₁ on the amplitude Finally, A₂ is the center, or mean, of the signal.

It is worth noting that, while we tried to use the third derivative of the Nernstian distribution, the fit was poor due to the fact that the satellite peaks of this distribution are not as sharp as those observed in the “true” signals.

The maximum amplitude of the central peaks of the third derivative of the modified Gaussian is a function of the A's, according to the expression

This value is obtained by evaluating the third derivative of the modified Gaussian at the zeroes of the fourth derivative of the modified Gaussian. The zeroes of the fourth derivative of the modified Gaussian are given by the expression

$${v}_{1 , 2 , 3 , 4} = {A}_{2} \pm \frac{\sqrt{2} \sqrt{3 \pm \sqrt{6}}}{2 {A}_{1}} \qquad ( 10 )$$

(These are the positions of the extremes of the third derivative of the modified Gaussian). The third derivative of the modified Gaussian is depicted in FIG. 4.

### Example 2

### “Nonlinear Lev-Mar Fit.vi”

The peak finder algorithm is an iterative method that finds the optimal set of Aₓ's and A_{y}'s that make equations (2) fit the X(V) and Y(V) components of the current vector. LabView has a vi called “Nonlinear Lev-Mar Fit.vi” that, given a data set, provides the optimal set of A's. This vi is the foundation upon which the algorithm is constructed.

Given X(V) and Y(V), and the fitting curves in (2), two error coefficients are defined as

$$\begin{matrix}{E}_{x} = {\sum }_{i} \frac{( {X}_{true} ( {v}_{i} ) - {{X}_{f i t} ( {A}_{xo} , {A}_{x1} , {A}_{x2} , {A}_{x3} , {A}_{x4} , {A}_{x5} , {A}_{x6} , {v}_{i} )}^{2}}{{σ}_{x i}^{2}} \\ {E}_{y} = {\sum }_{i} \frac{( {Y}_{true} ( {v}_{i} ) - {{Y}_{f i t} ( {A}_{yo} , {A}_{y1} , {A}_{y2} , {A}_{y3} , {A}_{y4} , {A}_{y5} , {A}_{y6} , {v}_{i} )}^{2}}{{σ}_{y i}^{2}}\end{matrix} \qquad ( 11 )$$

The standard deviations a give the weighting of points of the data set, and are usually set to 1. The optimum set of parameters (A's) will be such that the error coefficients are minimized. That happens when the gradients of the error coefficients equal zero.

$$\begin{matrix}∇ {E}_{x} = \frac{\partial {E}_{x}}{\partial {A}_{xn}} = - 2 {\sum }_{i} \frac{\frac{\partial {X}_{fit} ( {A}_{x} , {v}_{i} )}{\partial {A}_{xn}} ( {X}_{true} ( {v}_{i} ) - {X}_{fit} ( {A}_{x} , {v}_{i} )}{{σ}_{xi}^{2}} = 0 \\ ∇ {E}_{y} = \frac{\partial {E}_{y}}{\partial {A}_{yn}} = - 2 {\sum }_{i} \frac{\frac{\partial {Y}_{fit} ( {A}_{y} , {v}_{i} )}{\partial {A}_{yn}} ( {Y}_{true} ( {v}_{i} ) - {Y}_{fit} ( {A}_{y} , {v}_{i} )}{{σ}_{y i}^{2}} = 0\end{matrix}$$

Expanding the gradients in (12) in a Taylor series we obtain the matrix equations

∇Eₓ(Aₓ)=∇Eₓ(Aₓ₋ᵢₙᵢₜᵢₐₗ)+∇∇Eₓ(Aₓ₋ᵢₙᵢₜᵢₐₗ)(Aₓ₋ᵢₙᵢₜᵢₐₗ−Aₓ)=0

∇E_{y}(A_{y})=∇E_{y}(A_{y=initial})+∇∇E_{y}(A_{y-initial})(A_{y-initial}−A_{y})=0 (13)

That can be expressed as

$${\sum }_{l = 0}^{8} {a}_{kl} δ {A}_{1} = {β}_{k} \qquad ( 14 ) )$$

The Levenberg-Marquardt method incorporates a dimensionless parameter λ to the diagonal of matrix α to speed up convergence. The new matrix is then defined by

a′ⱼⱼ≡aⱼⱼ(1+λ)for k≠j

a′ₖⱼ≡aₖⱼ (15)

The system of equation is solved by a Newton-Raphson iterative scheme. The method converges to the optimal set of A's provided that a good initial guess is used. This is the basic step of our algorithm. A deeper explanation of this method can be found on “Numerical Recipes for C”

### Algorithm

This application may be used to read in and analyze any 4^{th} harmonic scan created by any version of DAQ-o-Matic. If the scan is not a 4^{th} harmonic scan, the application generates an error code (−111) and performs no further analysis. The user may define, via the Constants screen, a portion (in millivolts) of a scan to be analyzed by the application; however, the default is to analyze the entire scan.

After the data is read in, the application first attempts to find a “good fit” for X. A “good fit” is determined by a number of parameters including, but not limited to, a minimal mean square error (MSE) between the “true” scan and the “best fit” (see Discrimination Procedure). At present the application first attempts to fit X at 0 degrees. If this fit is a “bad” fit (e.g., high MSE), the application then attempts to fit X at 45 degrees. If this too is a “bad” fit, the application is unable to find a signal (peak) in X and, at present, is unable to solve for Ip or Eo. Under these conditions, the application generates an error code (−999) and performs no further analysis.

If a “good fit” is found for X, the application then attempts to find a “good fit” for Y. If, and only if, the application is able to find a “good fit” for X and Y at the same angle, will it continue to solve for Ip and Eo. At present, if the application is unable to find a “good fit” for X and Y at the same angle, it generates an error code (−999) and performs no further analysis. Page 9 out of

To determine a “good fit” for either X or Y, the application must first define an initial “guess” for the 9 coefficients used by the fitting algorithm. This initial guess must be made for both X and Y at each angle. Furthermore, this initial “guess” must be based upon the original data and the previously described characteristics of the 3^{rd} derivative of the Gaussian.

### Initial Guesses for X or Y at Every Phase

An initial 5th order polynomial is fit to the data using a technique known as Singular Value Decomposition (SVD). This polynomial is subtracted from the “original” data (be it X or Y), and the first and last 10 points are removed from this result. If we assume that the maximum and minimum of this curve correspond to the central peaks of the Gaussian, and that the positions of the central peaks are given by V₂ and V₃ in (10), we can then obtain a good initial guess for the fitting of the third derivative of the modified gaussian by

$$\begin{matrix}\begin{matrix}{A}_{2} = \frac{{v}_{2} + {v}_{3}}{2} \\ {A}_{1} = \frac{\sqrt{2 ( 3 - \sqrt{6}}}{\vert {v}_{3} - {v}_{2} \vert}\end{matrix} \\ {A}_{o} \frac{\vert {X}_{true} ( {v}_{3} ) - {X}_{true} ( {v}_{2} ) \vert}{7.8 {A}_{1}^{3}}\end{matrix} \qquad ( 16 )$$

It is worth noting that, in previous versions of this application, we used the following constant parameter values

Aₒ=1, A₁=14.5, A₂=200 mV, A₃=100,

A₄=100, A₅=100, A₆=100 (17)

However, these constant polynomial coefficients proved to be too large, forcing the method to converge from far away. Furthermore, when using these constant initial parameters, the method often failed due to the fact that it identified one of the satellite peaks of the signal as the central peak. T his “failure” was detected by checking if

$$\vert \frac{{X}_{true} ( {v}_{p2} ) - {X}_{fit} ( {v}_{p2} ) + {X}_{true} ( {v}_{p3} ) - {X}_{fit} ( {v}_{p3} )}{7.8 {A}_{o} {A}_{1}^{3}} \vert > K = 1 / 4 \qquad ( 18 )$$

If (18) was true, this indicated that the satellite peaks of the fit were separated from the true data by more that ¼ of the amplitude of the Gaussian fit. Under these conditions, we defined two parameters

ξ=sign[Aₒ{Xₜᵣᵤₑ(vₚ₂)−X_{fit}(vₚ₂)+Xₜᵣᵤₑ(vₚₛ)−X_{fit}(vₚ₃)}] (19)

$$D = \frac{\sqrt{2 ( 3 - \sqrt{6} )}}{{A}_{1}}$$

where D is was obtained from (10), and is the distance between the two central peaks of the third derivative of the modified Gaussian. We then attempted a new fit with the same initial conditions but with

Aₒ^{new}=−Aₒ^{old}

A₂^{new}=A₂^{old}+Dξ (20)

If this second fit failed or (18) was not true, then a third set of initial conditions was launched to fit the data. The third set of initial conditions was the same as the first with one exception: Aₒ=−1.

Having said all that, after a great deal of investigation, we have found that the new technique (16) of defining our initial “guess” based upon the “true” data minus a 5^{th} order polynomial is significantly better (in terms of speed and accuracy of conversion) than using constant parameters

Discrimination Procedure

As mentioned above, a number of criteria are used to determine if a set of calculated coefficients provides a “good fit” for either X or Y. These criteria, which are applied in a specific order for both X and Y, are as follows (in the order of application):

1) For a good fit, the difference between the “true” data and the fit must be minimal. Hence, we compute a weighted mean square error term, were the MSE is weighted by the amplitude of the Gaussian component of the data set⁴:

$$\begin{matrix}{MSE}_{weighted} = \frac{MSE}{{( Max ( {X}_{true} - {Y}_{{5}^{th} poly} ) - min ( {X}_{true} - {X}_{{5}^{th} poly} ) )}^{2}} \\ = \frac{{\sum }_{i = 1}^{n} {( {Y}_{true} - {Y}_{fit} )}^{2}}{{n ( Max ( {X}_{true} - {X}_{{5}^{th} poly} ) - min ( {X}_{true} - {X}_{{5}^{th} poly} ) )}^{2}}\end{matrix} \qquad ( 21 )$$

⁴This value is obtained by taking the difference between the maximum and the minimum of the data minus the preliminary 5^{th} order polynomial fit.

This weighted MSE error should be less than 0.001. If it is not, we redefine, as described above (15 & 16), some of the coefficients and re-fit the data.

2) For a “good fit,” the width of the gaussian term (A₁) is typically between 19 and 20. For example, from an experiment performed with 299 positive files from ab106 (may99), we obtained the following statistics on A₁

A, 128-4 5 (22)

A,, 136 oAl, 6 1

Hence, A₁ must be greater than 10 and less than 20 for any fit to be classified (considered) as a “good fit.”

3) If the fit has past the first two tests, than the weighted MSE must be less than 0.01. If either condition 2 or 3 fail, the application changes the angle (from 0 to 45) and attempts, once again, to satisfy all 3 criteria (1-3). As mentioned above, if the application is unable to satisfy all 3 criteria at 0 and 45 degrees for either X or Y, it is unable to solve for Ip and Eo (error code=−999).

4) If a “good fit” has been found for both X and Y (i.e., the fit for X and Y has passed criteria 1 through 3), then the application applies two final criteria: one to compare the fit for X to the fit for Y and one to compare the fit for R to the “true” R (scan). To compare the fit for X to the fit for Y, the application examines the difference between the calculated (A2ₓ and A2_{y}) Eo locations for X and Y. The absolute difference between these two values must be no greater than 50 mVolts. This value ensures that the fitting algorithm is not fitting the central peak to the satellite peaks of the data in either X or Y. The distance between peaks is given by the position of the extreme of the third derivative of the modified gaussian (4). The zeroes are at (6). It is worth noting that, given an average A, value of 14.5, the typical distance between the central peaks will be 70 mV; hence, the absolute difference between the Eos should never be greater than 50 mV.

After some experimentation, we noticed that an absolute difference between the Eos was greater than 50 mV in the case that the application fit (“locked-in”) to a “wrong” peak in either X or Y. For example, if X had a peak at 180 mV and one at 250 mV, the application may fit (find) the peak at 225 mV, causing the absolute difference in the Eos to be greater than 50 mV if the Eo for Y was found at 180 mV. To account for this case, if the absolute difference between the Eos is greater than 50 mV, we shift (via A₂), invert (Aₒ=−Aₒ) and re-fit the signal (X or Y) that is farthest from a user-defined expected Eo. The shift is in the direction of the expected Eo. If shifting and inverting improves (<weighted MSE) the fit, we use the newly found coefficients; otherwise, we return to the previous coefficients and report an Eo separation error (Error Code=−777).

5) To compare the fit for R to the true R (scan), we compute the Ip divided by the RMS of the fit

$$\frac{{I}_{p}}{\sqrt{{\sum }_{i} \frac{{( {R}_{true} - {R}_{fit} )}^{2}}{n}}} > K = 3.7$$

From empirical analysis, we have determined that this value should be greater than 3.70. If the Ip/RMS is less than 3.70, the application provides an error code of −888.

Solving for Ip and Eo

As previously noted, in this version of the application, both X and Y must be fit in order to solve for Ip and Eo (a positive result). The reason is that the amplitude in R is defined as

R(v)=\sqrt{X²(v)+Y²(v)} (23)

We have tried to extract the R amplitude from only one component (either X or Y alone) using the formula

$$R ( v ) = \frac{X ( v )}{cos ( θ )} \qquad ( 24 )$$

but since the phase shift is very noisy, we were not successful (24). Once we have fits for X and Y, the peak height (Ip or G′″ₘₐₓ) and center of the signal (Eₒ or A₂) are given by the following equations G.,-=4±36A,,Al³E 3.9A A,3

$${E}_{R}^{0} = \frac{{E}_{x}^{0} {I}_{x}^{2} + {E}_{y}^{0} {I}_{y}^{2}}{{I}_{x}^{2} + {I}_{y}^{2}}$$

If the application is able to calculate Ip and Eo with no errors, the traffic light will be green. If, on the other hand, the application is unable to calculate these values within the user-defined “settings” (Green/Yellow or Yellow/Red via the Constants control), then the traffic light will be yellow or red.

The final version of the application (LevMar.exe, Version 1.00a1) is located at the following location: Z:\Shared\New Peak Finder as a self-installing executable.

Output File

If this application is used to analyze multiple files (batch mode), at the completion of analysis, the application writes a tab-delimited spreadsheet file. A sample spreadsheet file appears in FIG. 2. There will be one row for each file processed by the application. The columns in this spreadsheet are labeled as follows: Filename, ScanDate, SampleDescription, Ip, Eo, Error Color, and Error Code. The first 3 columns are obtained directly from the file header. The Ip and Eo are, as mentioned above, calculated from the “best fit” minus background curve. As a reminder, if the application was unable to “fit” the scan, these values will be O and N/A. The Error Color is the color of the Error Traffic Light indicator which, if no error occurred, will always be green. In the event that an error occurred (i.e., the application was unable to fit the scan), the error color will either be yellow or red. Finally, the error code indicates the type of error, if any, that occurred during processing of the scan. At present, the possible error codes and their “interpretations” are as follows:

| Error Code | Interpretation |
|---|---|
| 0 | No error |
| −999 | Unable to fit X and/or Y |
| −888 | Low Ip/RMS Ratio Signal is too small to comfortably |
| | distinguish from noise |
| −777 | Large Eo separation (between Eoₓ and Eo_{y}) |
| −111 | Not a 4^{th} harmonic |

It is worth noting that if the error color is green, the error code will always be zero, and vise-versa. In addition, if the error color is yellow or red, the error code will always be nonzero. Finally, if for any scan, an error color and code are generated, the user should reexamine these scans on a file-by-file basis.

| Filename | ScanDate | SampleDescription | Ip | Eo | ErrorColor | ErrorCode |
|---|---|---|---|---|---|---|
| Y654_1.1- | 08/24/99 | Zip 1, Chip 1, | 0.00E + 00 | NaN | Red | −999 |
| 1 nm_001.cms | at | 1 nM, 15 min Chip | | | | |
| | 09:01:41 | 1 Pad 3 | | | | |
| Y654_1.1- | 08/24/99 | Zip 1, Chip 1, | 9.36E − 11 | 3.19E + 02 | Red | −777 |
| 1 nM_00.1 | at | 1 nM, 15 min Chip | | | | |
| cms | 09:01:55 | 1 Pad 5 | | | | |
| Y654_.1- | 08/24/99 | Zip 1, Chip 1, | 2.68E − 11 | 2.99E + 02 | Yellow | −888 |
| 1 nM_006.cms | at | 1 nM, 15 min Chip | | | | |
| | 09:02:51 | 1 Pad 9 | | | | |
| Y654_1.1- | 08/24/99 | Zip 1, Chip 1, | 8.19E − 11 | 1.43E + 02 | Green | 0 |
| 1 nM_007.cms | at | 1 nM, 15 min Chip | | | | |
| | 09:03:05 | 1 Pad 11 | | | | |

### Example 3

### Error Analysis

### Statistical Distribution of Fitting Parameters

We analyzed data from the protocol Dc800-Cyp2d Chip (40c) 100 hz with the program Lev-mar Fit 4p 4^{th}.vi without constrain parameters and found the following statistical data on the fitting parameters. The purpose of this statistical description of the fitting parameters is to generate synthetic signals with the same random characteristics as the real signals.

TABLE 1
Statistical data on fitting parameters found on dc800

| DC800 | mean a1 | stdev a1 | mean eO (v) | stdev eO(v) | # files |
|---|---|---|---|---|---|
| N6 homo | 13.868 | 0.282 | 0.13226 | 0.00386 | 3613 |
| N6 hetero | 13.68 | 0.4 | 0.13379 | 0.0045 | 1500 |
| w97 homo | 13.086 | 0.096 | 0.3075 | 0.00266 | 903 |
| w97 hetero | 12.93 | 0.32 | 0.31052 | 0.007 | 1500 |

### Example 4

### Two Potential Simulations using Peak finder Algorithms

We generated synthetic data files with two peaks, one on the W97 position and other to the right of the w97 peak. This second peak eas termed “other”. Both peaks were generated with a known Ip, and a₁ and eO following the distribution found for the homo w97 peaks on dc800 (Table 1). The synthetic peaks were run thought the peak finder and the compute Ip were used to estimate the uncertainties on the peak finder scheme. 100 files were randomly generated for every peak separation, and the standard deviation of the “other” peak computed. Only 2 potentials were enabled on Lev-Mar Fit 4p 4^{th}.vi.

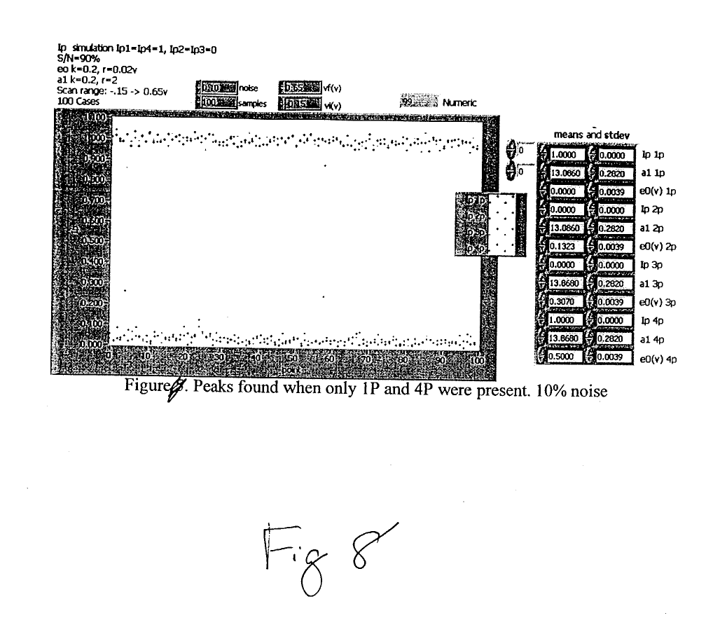

We present the 95% confdience uncertainty (2 standard deviations) n the Ip as a function of the “other” peak location⁵. FIG. 5 represents the uncertainty on the additional (“other”) peak as a function of its eO. We run 9 cases

1) Both Ip were equal to 1. Noise level=0

2) Both Ip were equal to 1. Noise level=0.1

3) Both Ip were equal to 1. oise level=0.2

4) Other Ip=0.2, W97 Ip=1, Nosie level=0

5) Other Ip=1, W97 Ip=0.2, Noise level=0

6) Other Ip=0.2, W97 Ip=1, Noise level=0.1

7) Other Ip=1, W97 Ip=0.2, Noise level 0.1

8) Other Ip=0.2, W97 Ip=1, Noise level=0.2

9) Other Ip=1, W97 Ip=0.2, Noise level=0.2. ⁵Leaving w97 eO=0.38v

### Conclusions

### Regarding the Two Potential Simulation

1) After 90 mV of separation, the uncertainty of both peaks is much smaller. There is a minimum on the uncertainty at 90 mV separation. This is a particularly good separation between two potential. 90 mV is also the distance between the central and the satellite peaks of a signal. This may be the reason for the minimum in uncertainty.

2) Noisy signals have more uncertainty.

3) A small signal has more uncertainty in the presence of a large signal.

4) A large signal has less and less uncertainty when the other signal is smaller.

### Example 3

### Four Potential Simulations Using Peak Finder Algorithms

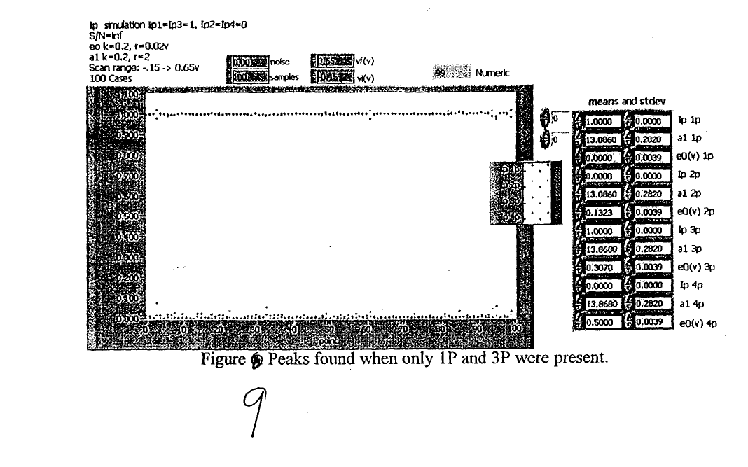

We generated sets of 100 synthetic data files with four peaks, with parameters following random normal distributions with means and standard deviations (see FIG. 6).

All peaks were generated with a random Gaussian distribution for al and eO following the distribution found on the dc800 experiment whenever possible. Since we didn't have statistical data for the potentials at OmV and 500 mV, we used the values shown in FIG. 6. The Ips values changed depending on the case. The synthetic peaks were run thought the peak finder and the computed Ip were used to estimate the uncertainties on the peak finder scheme for every of the four peaks. Three simulations were done:

1) Four potentials simulation. In this simulation, the peaks at (OmV) and (500 mV) were generated with Ip=1. The generated N6 and W97 had Ip=O. We were most interested in estimating how large can the program call a peak that in reality is not there.

2) Four potentials simulation, 1 p and 3p on, 2p and 4p off. In this simulation, the peaks at (OmV) and W97 were generated with Ip=1. The generated N6 and (500 mV) had Ip=0. We were most interested in estimating how large can the program call a peak in reality is not there.

3) 4 potential simulations for increasing peak sizes. In this simulation, the peaks at (OmV) and W97 were generated with Ip=1. The generated N6 and (500 mV) had Ips ranging from 0.1 to 1. We were most interested in estimating the absolute uncertainties for N6 and (500 mV).

Four Potentials Simulation, 1p and 4p on, 2p and 3p Off

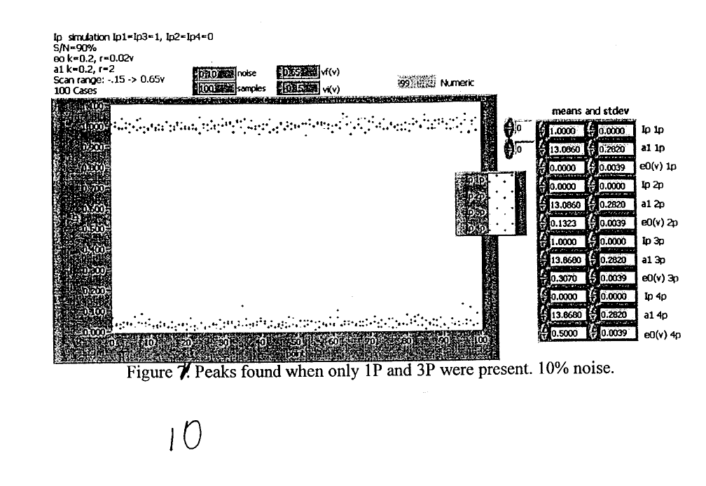

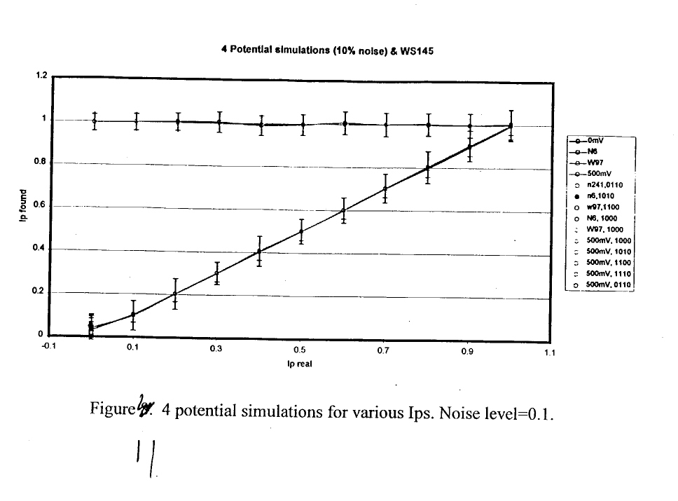

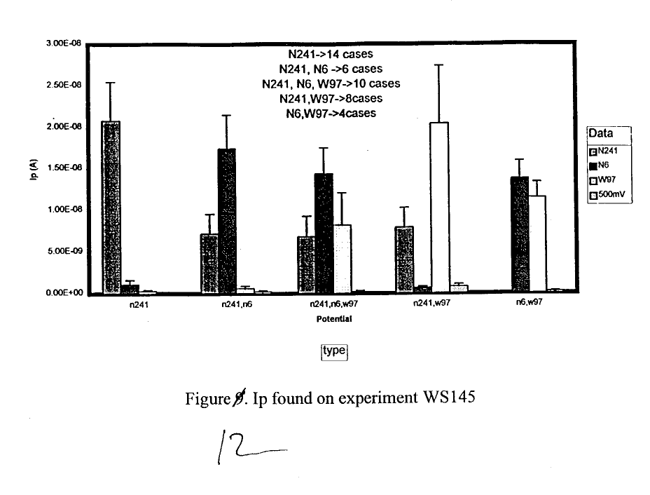

We generated synthetic data files with peaks 1 and 4 having an lp=1 and peaks 2 and 3 having lp=0. The four potential peak finder was run and the peak size found is presented on FIGS. 7 and 8. The noise level used was 0% and 10%. The uncertainty is shown in tables 3 and 3A.

TABLE 3
Uncertainties (2 × Stdev) on 4 potential detection,
when 1P = 4P = 1, 2P = 3P = 0. 0% noise

| | |
|---|---|
| mean 1p 0.998 | 2 × stdev 1p 0.007 |
| mean 2p 0.016 | 2 × stdev 2p 0.039 |
| mean 3p 0.004 | 2 × stdev 3p 0.010 |
| mean 4p 0.997 | 2 × stdev 4p 0.014 |

TABLE 3a
Uncertainties (2 × Stdev) on 4 potential detection,
when 1P = 4P = 1, 2P = 3P = 0. 10% noise

| | |
|---|---|
| mean 1p 0.999 | 2 × stdev 1p 0.038 |
| mean 2p 0.037 | 2 × stdev 2p 0.043 |
| mean 3p 0.038 | 2 × stdev 3p 0.068 |
| mean 4p 0.995 | 2 × stdev 4p 0.053 |

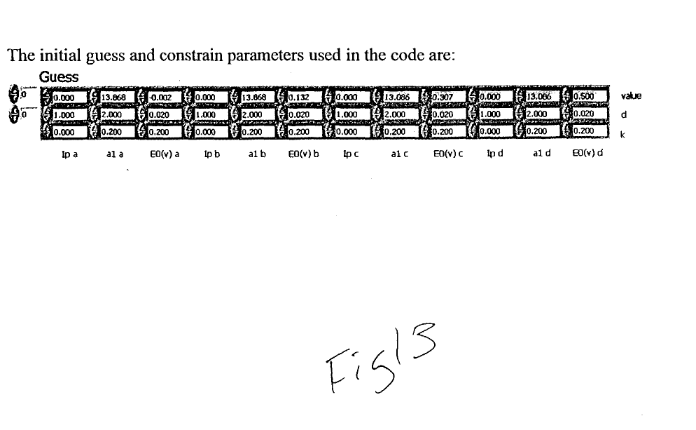

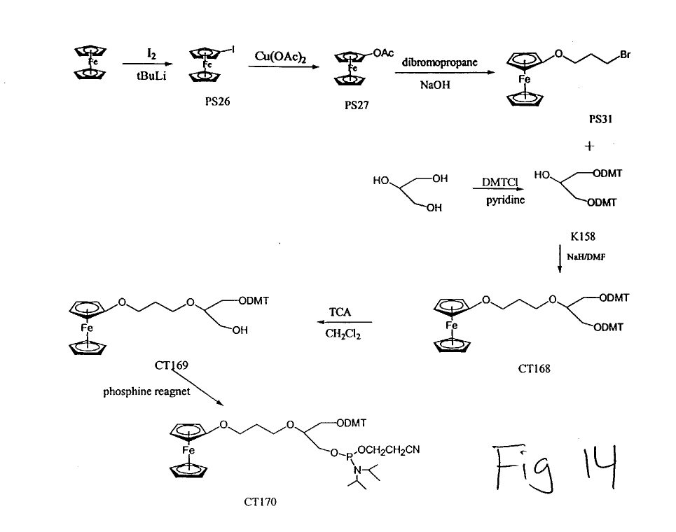

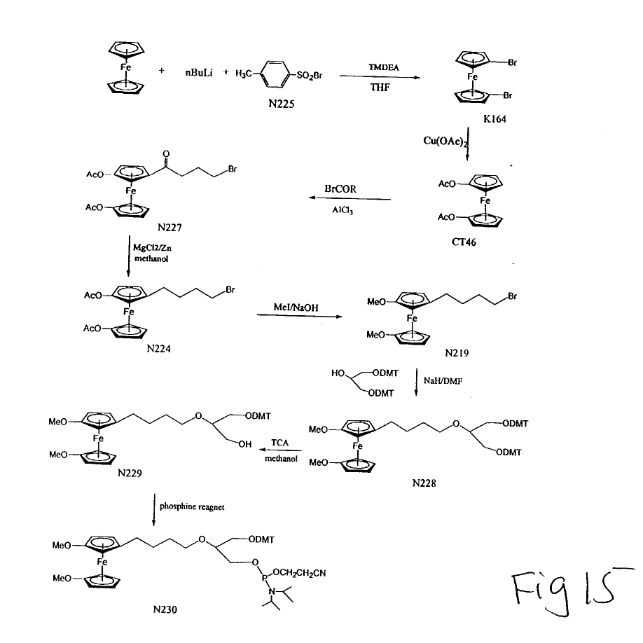

We generated synthetic data files with peaks 1 and 3 having an Ip=1 and peaks 2 and 4 having Ip=0. The four potential peak finder was run and the peak size found is presented on FIGS. 9 and 10. The noise level used was 0%. Uncertainties are shown in Tables 4 and 5.

TABLE 4
Uncertainties (2 × Stdev) on 4 potential detection,
when 1P = 3P = 1, 2P = 4P = 0. 0% noise

| | |
|---|---|
| mean 1p 0.998 | 2 × stdev 1p 0.009 |
| mean 2p 0.011 | 2 × stdev 2p 0.025 |
| mean 3p 1.000 | 2 × stdev 3p 0.011 |
| mean 4p 0.003 | 2 × stdev 4p 0.007 |

TABLE 5
Uncertainties (2 × Stdev) on 4 potential detection,
when 1P = 3P = 1, 2P = 4P = 10% noise

| | |
|---|---|
| mean 1p 0.995 | 2 × stdev 1p 0.038 |
| mean 2p 0.040 | 2 × stdev 2p 0.045 |
| mean 3p 0.997 | 2 × stdev 3p 0.042 |
| mean 4p 0.030 | 2 × stdev 4p 0.026 |

### Conclusions

The results from the simulations performed on 4 potential are summarized on Table6. These simulations represent 2 noise levels (0% andl0%). Also, two configurations are simulated. In the first one: (1001) the Ips of the first and fourth potentials are equal to 1, while the second and third are equal to 0. in the second one: (1010) the Ips of the first and third potentials are equal to 1, while the second and fourth are equal to 0. The simulations estimate the error that we are likely to encounter when we allow the fitting routine to adjust 4 potentials when only two are present.

TABLE 6
4 potential simulations results

| | 1001-0% | | 1001-10% | | 1010-0% | | 1010-10% | |
|---|---|---|---|---|---|---|---|---|
| | Mean | 2 × stdev | Mean | 2 × stdev | Mean | 2 × stdev | Mean | 2 × stdev |
| OmV | 0.998 | 0.007 | 0.999 | 0.038 | 0.998 | 0.009 | 0.995 | 0.038 |
| N6 | 0.016 | 0.039 | 0.037 | 0.043 | 0.011 | 0.025 | 0.04 | 0.045 |
| W97 | 0.004 | 0.01 | 0.038 | 0.068 | 1 | 0.011 | 0.997 | 0.042 |
| 500 mV | 0.997 | 0.014 | 0.995 | 0.053 | 0.003 | 0.007 | 0.03 | 0.026 |

The main conclusions are:

1) When we generate 2 peaks of size “1” without noise, and try to detect four, the peaks that are present have an uncertainty for 95% confidence of up to 1.4% of the original peak size. This uncertainty is computed by RMSing the mean error and two standard deviations for every case.

u_{95%}=\sqrt{(1−mean)²+(2_{—}x_{—}stdeve)²}

u_{95%}_{_{—}}₁₀₀₁_{_{—}}_{10%}_{_{—}}_{OmV}=({square root over (1−0.999)²+(0.038))}²=0.038

u₉₅%_{_{—}}₁₀₁₀₋₁₀%_{_{—}}_{500OmV}=\sqrt{(1−0.995)₂+(0.053)²}=0.038

u_{95%}_{_{—}}₁₀₁₀_{_{—}}_{10%}_{_{—}}_{OmV}=\sqrt{(1−0.095)²+(0.038)²}=0.038

u_{95%}_{_{—}}_{1010-10%}_{_{—}}_{w97}=\sqrt{(1−0.0997)²+(0.042)²}=0.042 (26)

2. When we generate 2 peaks of size “1” without noise, and try to detect four, the peaks that are not present have an uncertainty for 95% confidence of up to 4% of the present original peak size (4%×1=0.04). This uncertainty is computed by RMSing average of the mean and two standard deviations for every case.

u_{95%}=\sqrt{(mean)²+(2_{—}x_{—}stdev)²}

u_{95%}_{_{—}}_{1001-0%}_{_{—}}ᵤ₆=\sqrt{(0.016)²+(0.039)²}=0.042

u_{95%}_{_{—}}_{1001-0%}_{_{—}}_{w97}=\sqrt{(0.004)²+(0.01)²}=0.011

u_{95%}_{_{—}}_{1010-0%}_{_{—}}ᵤ₆=({square root over (0.011)²+(0.025)²)}=0.027

u_{95%}_{_{—}}_{1010-0%}_{_{—}}_{500mV}=(0.003)²+(0.007)²=0.008 (27)

In real cases where only two peaks are present, they are rarely of the same size. In order to estimate the 95% confidence uncertainty, we can take the average of the present 2 peaks, and compute a 4% of that.

3. When we generate 2 peaks of size “1” with noise levels of 0.1 (similar to the noise level of a 2nA signal), and try to detect four, the peaks that are present have an uncertainty for 95% confidence of up to 5.3% of the original peak size. This uncertainty is computed by RMSing the average of the mean error and two standard deviations for every case.

u_{95%}=\sqrt{(1−mean)²+(2_{—}x_{—}stdev)²}

u_{95%}_{_{—}}_{1001-10%}_{_{—}}_{0mV}=\sqrt{(1−0.999)²+(0.038)²}=0.038

u_{95%}_{_{—}}_{1001-10%}_{_{—}}_{0mV}=\sqrt{(1−0.995)²+(0.053)²}=0.053

u_{95%}_{_{—}}_{1010-10%}_{0mV}=\sqrt{(1−0.995)²+(0.038)²}=0.038

u_{95%}_{_{—}}_{1010-10%}_{_{—}}_{w97}=\sqrt{(1−0.997)²+(0.042)²}=0.042

4. When we generate 2 peaks of size “1” with noise level 0.1, and try to detect four, the peaks that are not present have an uncertainty for 95% confidence of up to 7.8% of the present original peak size (7.8%×1=0.078). This uncertainty is computed by taking the geometric average of the mean and two standard deviations for every case.

u_{95%}=\sqrt{(mean)²+(2_{—}x_{—}stdev)²}

u_{95%}_{_{—}}_{1001-10%}_{_{—}}ᵤ₆=\sqrt{(0.037)²+(0.043)²}=0.057

u_{95%}_{_{—}}_{1001-10%}_{_{—}}₂₉₇=\sqrt{(0.038)²+(0.068)²}=0.078

u_{95%}_{_{—}}_{1010-10%}_{_{—}}ₙ₆=\sqrt{(0.040)²+(0.045)²}=0.060

u_{95%}_{_{—}}_{1010-10%}_{_{—}}_{300mL}=\sqrt{(0.03)²+(0.026)²}=0.04 (28)

4 Potential Simulations for Increasing Peak Sizes

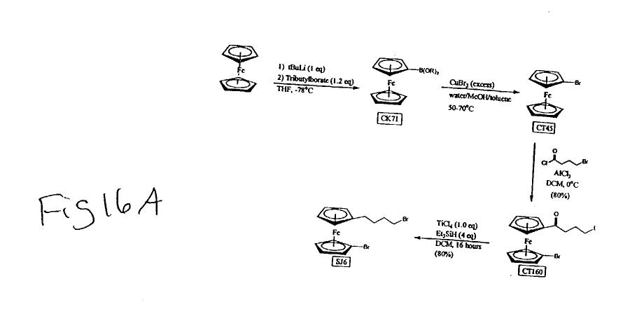

We generated synthetic data files with peaks 1 (OmV) and 3 (w07) having an Ip=1 and peaks 2 (N6) and 4 (500 mV) having Ip ranging from 0.1 to 1. The four potential peak finder was run and the peak sizes found are presented on FIG. 11. The noise level used was 10%. The distribution on the random parameters are the same used in previous simulations (FIG. 6).

The real peaks of the N6 and 500 mV potentials are presented on the x-axis, while the average peaks found are presented on the y-axis. Error bars represent 2×standard deviations of the set of 100, or 95% confidence uncertainty. In this simulations (OmV) and W97 had in reality always Ip=1. So the peaks found were always close to 1. The uncertainties for the (OmV) potential were always close to 0.05, for N6 were 0.06, for w97 were 0.043 and for the (500 mV) potential the uncertainties were 0.039. Table 7, presents the same information as FIG. 11.

TABLE 7
4 P simulations for various Ip sizes. Noise level = 0.1.

| | | | | | 2 × | 2 × | 2 × | 2 × |
|---|---|---|---|---|---|---|---|---|
| | mean | mean | mean | mean | stdev | stdev | stdev | stdev |
| Ip2, Ip4 | OmV | N6 | W97 | 500 mV | OmV | N6 | W97 | 500 mV |
| 0.1 | 0.999 | 0.1 | 0.999 | 0.104 | 0.038 | 0.068 | 0.042 | 0.035 |
| 0.2 | 1.002 | 0.203 | 0.997 | 0.203 | 0.04 | 0.071 | 0.041 | 0.036 |
| 0.3 | 1.003 | 0.301 | 0.996 | 0.301 | 0.048 | 0.052 | 0.037 | 0.039 |
| 0.4 | 0.99 | 0.406 | 0.995 | 0.401 | 0.046 | 0.069 | 0.043 | 0.037 |
| 0.5 | 0.995 | 0.501 | 0.997 | 0.5 | 0.048 | 0.057 | 0.047 | 0.041 |
| 0.6 | 1.006 | 0.601 | 0.999 | 0.6 | 0.053 | 0.059 | 0.04 | 0.039 |
| 0.7 | 1 | 0.703 | 1 | 0.7 | 0.054 | 0.067 | 0.043 | 0.039 |
| 0.8 | 1.001 | 0.803 | 0.997 | 0.796 | 0.052 | 0.074 | 0.047 | 0.04 |
| 0.9 | 0.997 | 0.903 | 0.996 | 0.896 | 0.055 | 0.071 | 0.04 | 0.04 |
| 1 | 1.004 | 0.999 | 0.997 | 0.995 | 0.069 | 0.071 | 0.045 | 0.04 |

Also, on FIG. 11, it is presented experimental data from WS145, on protocols cf-dc844-93 Hz. The chips information follows on Table 8.

Methods for Testings Sps with Low Potential.

WS145: Chip Plan

Hybridization (HYB) Buffer:

Make sol'n for 1 Hyb vol for each one. Number of chip 20

| Reagents | Vol (ul) | Stock | *Conc. |
|---|---|---|---|
| LC100801_2 Untreated Hyb buffer | 1424.00 | | |
| FBS Lot#1105277 | 160.00 | 10% | 1.42% |
| Fresh 100 mM C6 | 16.00 | 100 mM | 1.43 mM |
| Total Vol. | 1600.00 | | |

*Final conc. On chip for TM = 50 nM for each TM

SP=125 nM/ea (1:400 dil)

Add 80 ul of the hyb buffer into each tube and add 40 ul of TM_H₂O into each corresponding tube.

Machine Used: esensor4800 (ID#103026)

Data saved at Data/hydra/cf-dc844-93 HzR 117 h/Into8/Ex10/R560/G551d/Feb/05/02/wenmeishi Chip DC857 was used and data was scanned at 1, 2, 3 & 4^{th} harmonic with Javier's help

TABLE 8
Chip information from experiment WS145

Chip

| Arrangement | | Scan Protocol | Expect Signals |
|---|---|---|---|
| | Exon, 4 | | |
| ws145-01 | D3166 + N277 | Cf-dc844- | N241 (low potenial, Eo-omV) |
| ws145-02 | D3166, D3167 + N277, D3250 | 93HzR117h | N242 W97 |
| ws145-03 | D3166, D3167 + D3265, D3250 | | N6, W97 |
| | Intron 8, | | |
| ws145-04 | D3481 + N270 | Cf-dc844- | N241 |
| ws145-05 | D3480, D3481 + D3792, N270 | 93hzInto8 | N241, N6 |
| ws145-06 | D3480, D3481, D3482 + D3792, N270, | | N241, N6, W97 |
| | D3966 | | |
| | Exon 10, | | |
| ws145-07 | D3105 + N272 | Cf-dc844- | N241 |
| ws145-08 | D3104, 3105 + D3133, N272 | 93hzEx10 | N241, N6 |
| ws145-09 | D3104, D3105, D3221 + D3133, N272, | | N241, N6, W97 |
| | D3212 | | |
| | Exon 11, | | |
| ws145-10 | D3114 + N278 | Cf-dc844- | N241 |
| ws145-11 | D3111, D3114 + N278, D4106 | 93hzR560 | N241, W97 |
| ws145-12 | D3111, D3114 + D4105 D4106 | | N6, W97 |
| ws145-13 | D3112 + N271 | Cf-dc844- | N241 |
| ws145-14 | D3111, D3112, D3113 + N271, D4219, | 93hzG551d | N241, W97 |
| | D4221 | | |
| ws145-15 | D3111, N269 | | N241, N6, W97 |
| ws145-16 | D3113, N269 | | N241 |
| ws145-17 | D3111, D3113 + N269, D4221 | | N241, W97 |
| ws145-18 | D3111, D3112, D3113 + N269, D4218, | | N241, N6, W97 |
| | D4221 | | |

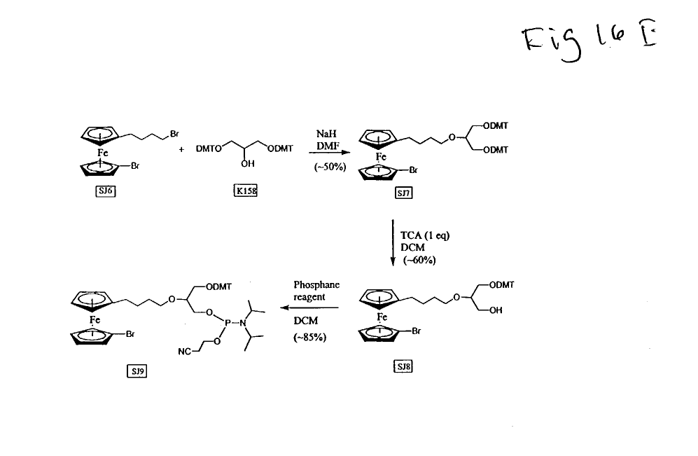

We separated the positive pads from experiment WS145 into 5 categories, depending on the potentials that were present. FIG. 12 shows the relative sizes of the unreal peaks pulled by the program, compared to the real peaks. The relative uncertainty for 95% confidence, computed as (2) is presented in Table 9.

TABLE 9
Uncertainties on experiment WS145.

| | n241 | n241, n6 | n241, n6, w97 | n241, w97 | n6, w97 |
|---|---|---|---|---|---|
| N241 | | | | | 0.97% |
| N6 | 7.42% | | | 5.05% | |
| W97 | 1.79% | 6.68% | | | |
| 500 mV | 0.94% | 2.76% | 3.75% | 7.04% | 2.00% |

### Conclusions

1) The absolute uncertainties on simulations (error bars on FIG. 11) are very similar for a particular label, almost independent on the Real Ip.

2) Simulations show that N6 has larger uncertainties than the potential at 500 mV, probably due to two reasons. N6 is sandwiched between two other peaks while the potential at 500 mV is only close to one. Second, the potential at 500 mV is farther from W97 than N6 is from either W97 or the potential at (OmV).

3) The absolute uncertaintie's on simulations for the four labels are consistently below 0.06, or 6%.

4) Experiment WS145 is consistent with the simulations, showing that when the program detects a peak that is not there, the lp pulled is consistently 7.5% (close to 6%) of the average of the real peaks.

### Example 4

### Preparation of Ferrocene Derivatives with Multiple Redox Potentials

Alkoxy Ferrocene Derivatives with Mono-Alkoxy Groups.

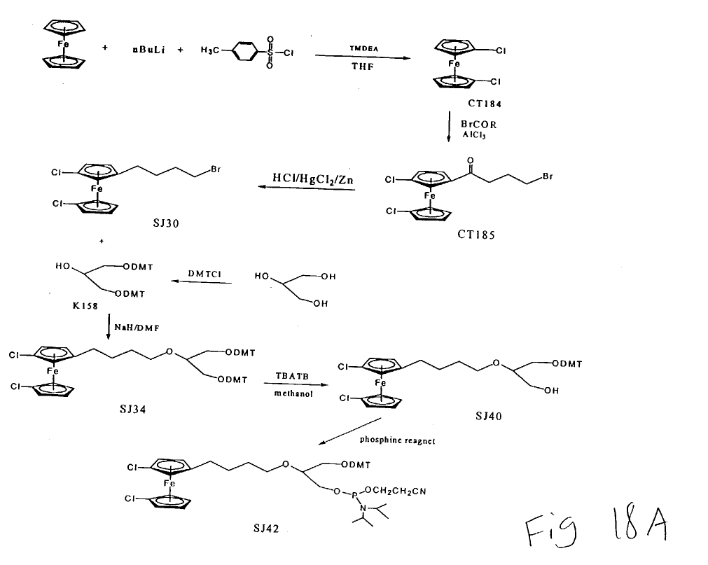

FIG. 14 depicts a scheme for synthesizing CT170.

Synthesis of CT170. To a solution of CT169 (0.86 g, 1.35 mmol) in dichloromethane (30 mL) was added C96 (230 mg, 1.35 mmol). The mixture was cooled to 0° C., and N,N,N′N′-tetraisopropylamino, 2-cyanoethoxy phosphane (1.3 mL, 1.22 g, 4.05 mmol) was added. The reaction mixture was warmed up to room temperature and stirred for 2 hours at room temperature. The mixture was diluted in 60 mL of dichloromethane, extracted by waster three times, dried over sodium sulfate and concentrated. The crude product was purified on a silica gel column packed with 1% TEA in hexane, and eluted with 1%TEA & 5-15% ethyl acetate in hexane to yield the desired product CT170 as a yellow sticky oil (0.92 g, 81%). The product was dissolved in acetonitrile, and was filtered through a 0.25 pm filter, and then was concentrated. Anal. Calcd. for C₄₆H₁₇N₂O₇PFe: 836.33. Found: 836.

Alkoxy Ferrocene Derivatives with Dialkoxyl Groups

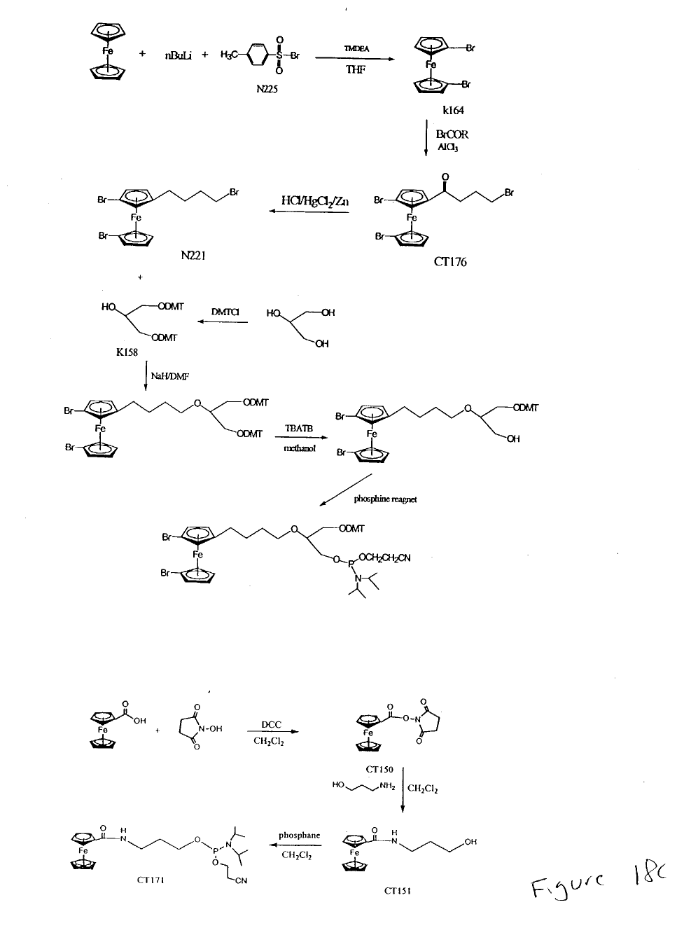

FIG. 15 depicts a synthetic scheme for the synthesis for several alkoxy ferrocene derivatives substituted with dialkoxyl groups.

Synthesis of N225. To a solution of toluenesulfinic acid (175.0 g, 0.98 mol.) in water (600 mL) slowly added bromine in cold methanol until the orange color persisted. More toluenesulfinic acid solution was added to change the color from orange to slightly yellow. The precipitate was filtered, washed by water. The solid was passed through a short silica gel column with dichloromethane. The crude product was purified on a column of 300 g of silica gel eluted by dichloromethane to yield 134.6 g of N225 (69%). ¹H NMR (300 MHz, CDCl₃) 7.87 (d, 2H), 7.30 (d, 2H), 2.49 (s, 3H).

Synthesis of K164. To a solution of ferrocene (30.0 g, 0.16 mol.) in ethyl ether (1 L) added n-butyl lithium (220 mL of 1.6 M in hexane) and tetramethylethylenediamine (27.0 mL, 0.18 mol.), and the solution was purged by argon for 10 min., then was stirred at room temperature overnight. The mixture was cooled to −78° C., and N225 (90.0 g, 0.38 mol.) was added. The reaction mixture was maintained at this temperature for 1 hour, then slowly warmed up to room temperature, and was stirred an additional 30 min. before being quenched by 30 mL of water. The mixture was filtered, and the solid was extracted by hexane several times. The combined organic layers were extracted by water, dried over sodium sulfate, and concentrated. The crude product was purified on a column of 400 g of silica gel eluted by hexane to provide the desired product K164 (40.0 g, 72%). The product could be further purified by recrystallization from methanol. GC/MS: m/e 346 (30), 344 (63), 342 (36), 128 (100), 102 (13).

Synthesis of CT46. To a solution of K164 (20.0 g, 59.2 mmol.) in ethanol (1 L) added copper (II) acetate (58.0 g, 0.29 mol), and the mixture was purged by argon for 10 minutes. The reaction was heated at reflux for 40 min., and then was cooled to room temperature. The mixture was extracted by ethyl ether several times. The organic layers were washed with water, brine, dried (NaSO₄) and concentrated. The crude product was purified on a column of 200 g of silica gel, packed in 1% TEA in hexane, and was eluted by 5-10% ethyl acetate in hexane to yield CT46 (8.2 g, 46%). GC/MS: m/e 348 (100), 311 (10), 183 (26), 128 (46).

Synthesis of N227. To a solution of CT46 (1.0 g, 3.3 mmol.) in dichloromethane (15 mL) added bromobutyryl chloride (0.56 mL, 5.0 mmol.) and aluminum chloride (1.32 g, 10.0 mmol), and the reaction was maintained at room temperature for 30 min., then was quenched in cold 5% NaOH in water. The mixture was extracted by ethyl ether, and the combined organic layer was extracted by water, dried over sodium sulfate and concentrated. The product was used in the next reaction without further purification. GC/MS: m/e 370 (39), 328 (19), 286 (100), 207 (42), 179 (12).

Synthesis of N224. To a solution of N227 (12.0 g, 26.7 mmol.) in toluene added zinc (180 g), mercury chloride (189) and water (350 mL), then 350 mL of concentrated HCl was slowly added. The mixture was stirred at room temperature for 35 min., and then was filtered. The aqueous layer was extracted by hexane three times, and the combined organic layers were washed with water, brine, dried over sodium sulfate and concentrated. The crude product was purified on a column of 200 g of silica gel packed in 1% TEA of hexane, and eluted by 5-10% ethyl acetate in hexane to yield the desired product N224 (8.0 g, 77%). GC/MS: m/e 438 (25), 436 (26), 396 (27), 394 (29), 354 (93), 352 (100), 272 (20), 179 (25).

Synthesis of N219. A solution of N224 (8.0 g, 18.3 mmol.) in a mixture of dioxane (90 mL) and methanol (10 mL) was purged by argon for 10 min. Then to the mixture was added a solution of NaOH (3.68 g, 92.0 mmol.) in water (21 mL) in the darkness. The mixture was stirred at room temperature for 10 min., then methyl iodide (11.2 mL) was added and the reaction mixture was stirred for 3 hours at room temperature. And an additional of 50 ml water was added into the mixture, which was extracted by hexane in several times. The combined organic layers were extracted with water, dried over sodium sulfate and concentrated. The crude product was purified on a column of 200 g of silica gel, packed in 1% TEA in hexane, and eluted by 1-2% ethyl acetate/hexane to afford the desired product N219 (5.0 g, 72%). ¹H NMR (300 MHz, CDCl₃) 4.08 (m, 4H), 3.81 (m, 3H), 3.64 (d, 6H), 3.40 (t, 2H), 2.23 (t, 2H), 1.87 (m, 2H), 1.62 (m, 2H). GC/MS: m/e 382 (92), 380 (100), 300 (64), 149 (23), 121 (26).

Synthesis of N228. To a solution of 1,3-diDMT glycerol (17.76 g, 25.5 mmol.) in DMF (80 mL) was added NaH (60% in mineral oil, 1.02 g, 25,5 mmol.). After the mixture was stirred at room temperature for 15 min., a solution of N219 (4.84 g, 12.74 mmol.) in DMF (20 mL) was added, and the reaction mixture was stirred at room temperature overnight. The mixture was diluted with 700 mL of ethyl acetate, and then extracted by water. The organic layer was dried over sodium sulfate and concentrated. The crude product was purified on a column of 250 g of silica gel packed in 1%TEA in hexane, and eluted by 1% TEA & 10-20% of dichloromethane in hexane to yield the desired product N228 (7.0 g, 54%). ¹H NMR (300 MHz, CDCl₃) 6.76-7.30 (m, 26H), 4.08 (broad, 4H), 3.80 (m, 15H), 3.61 (m, 7H), 3.50 (t, 2H), 3.22 (m. 4H), 2.20 (broad, 2H), 1.58 (m, 4H); Anal. Calcd for C₆₁H₆₄FeO₉: 996. Found: 996.

Synthesis of N229. To a solution of N228 (7.0 g, 7.03 mmol.) in dichloromethane (400 mL) was added trichloroacetic acid (1.15 g, 7.03 mmol) in dichloromethane (100 mL), and the mixture was stirred at room temperature for 3 min., and was quenched by 10 mL of TEA and 40 mL of methanol. The mixture was extracted by water, dried over sodium sulfate and concentrated. The crude product was purified on a column of 250 g silica gel packed in 1% TEA in hexane, and eluted by 1%TEA & 10-30% ethyl acetate in hexane to yield the desired product N229 (1.7 g, 71% yield based on consumed starting material) and the recovered starting material (3.3 g). ¹H NMR (300 MHz, CDCl₃) 6.76-7.30 (m, 13H), 4.05 (broad, 4H), 3.30-3.81 (m, 18H), 3.22 (m, 4H), 2.01(m, 2H), 1.58 (m, 4H); Anal. Calcd. for C₄₀H₄₆FeO₇: 694. Found: 694.

Synthesis of N230. To a solution of N229 (1.7 g, 2.62 mmol.) in dichloromethane (20 mL) was added DIPEA (2.27 mL, 13.10 mmol.) and C96 (0.90 g, 5.24 mmol.). The mixture was cooled to 0° C., and N,N,N′N′-tetraisopropylamino, 2-cyanoethoxy phosphane (2.16 mL, 6.54 mmol.) was added. The reaction mixture was warmed up to room temperature and stirred for 2 hours at room temperature. The mixture was diluted in 80 mL of dichloromethane, extracted by waster three times, dried over sodium sulfate and concentrated. The crude product was purified on a column of 80 g of silica gel packed in 1% TEA in hexane, and eluted by 1%TEA & 5-15% ethyl acetate in hexane to yield the desired product N230 (1.5 g, 75%). The product was dissolved in acetonitrile, and was filtered through a 0.25 um filter, and then was concentrated. The coupling efficiency of N230 from DNA synthesizer was 96%. ¹H NMR (300 MHz, CDCL3) 6.70-7.30 (m, 13H), 4.18 (broad, 4H), 3.50-3.80 (m, 24H), 3.16 (d, 2H), 2.50 (m, 4H), 1.58 (m, 4H), 1.10 (m, 12H). Anal. Calcd. for C₄₉H₆₃N₂O₈Pfe: 894. Found: 894.

Mono-Halogenated Ferrocene Derivatives

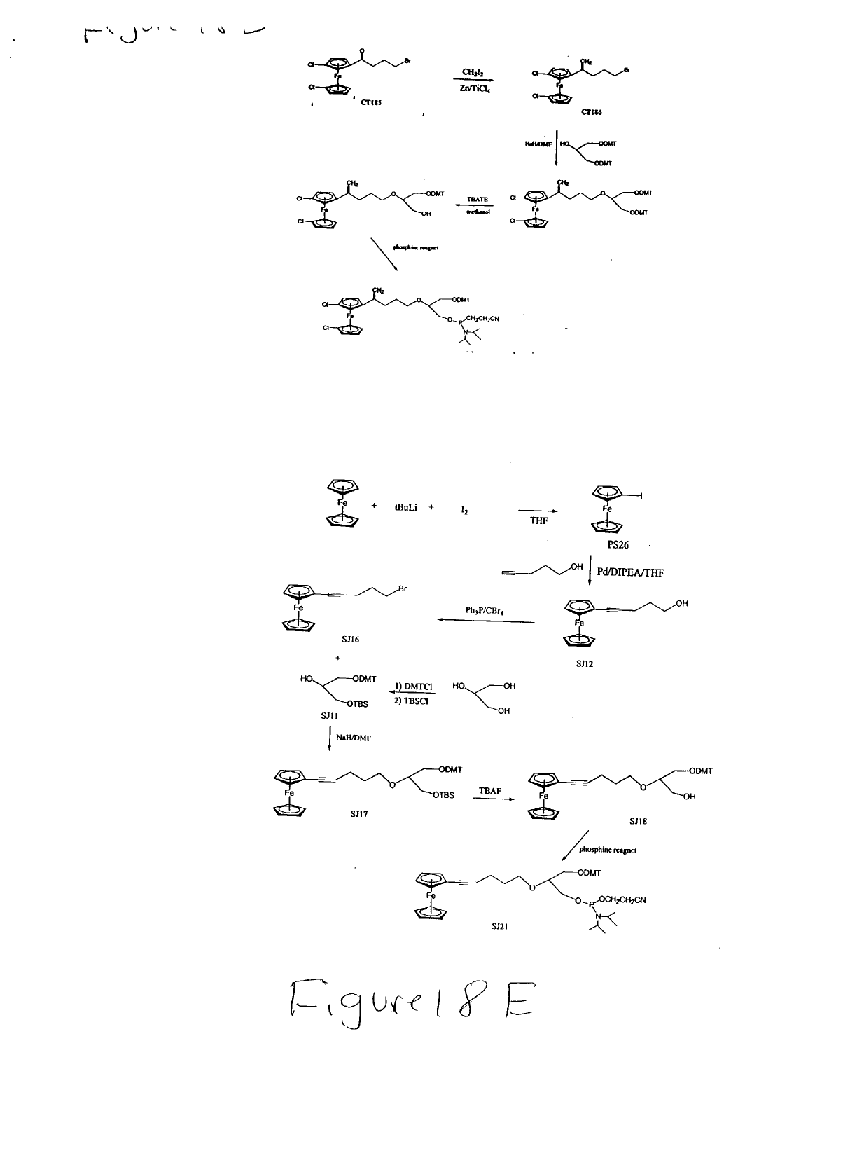

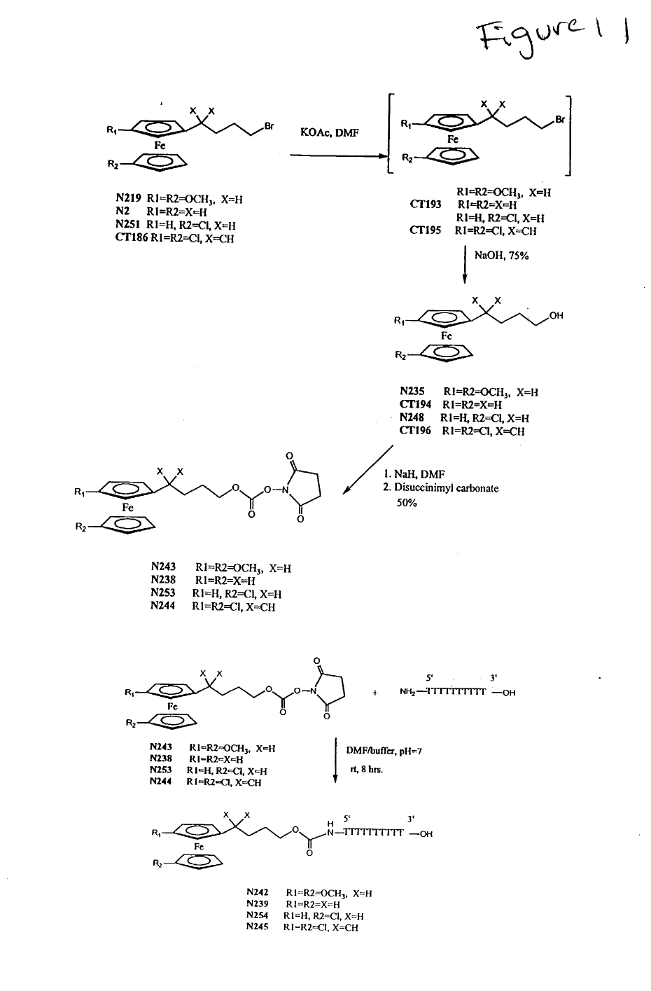

FIGS. 16A through C depict various synthetic schemes for the synthesis of mono halogenated ferrocene derivatives described below.

Synthesis of CK71. A solution of 71.1 g (0.38 moles) of ferrocene in 360 mL of dry THF was cooled to 0° C. A 1.7-M solution of tert-butyllithium in pentane (225 mL, 0.38 moles) was added dropwise, and the mixture was stirred for 10 minutes at 0° C. and warmed to room temperature over 40 minutes. The mixture was cooled to −78° C., and 123 mL (105 g, 0.45 moles) of tributylborate was added dropwise. After 10 minutes at −78° C., the reaction mixture was warmed to room temperature and stirred for 2 hours. The solution was then cooled to 0° C., and the reaction was quenched with the addition of 180 mL 5% (v/v) conc. HCl in water. Ether (250 mL) was added, and the mixture was filtered through Celite. The organic layer was separated, and the aqueous layer was extracted with ether. The combined organic layers were washed with brine and concentrated to a brown oil. The crude product was purified by pad-filtration on a silica gel pad, and eluted with hexanes to produce only unreacted ferrocene, and subsequent eluted with 50% ethyl acetate in hexanes to give 40.6 g of CK71 as a mixture of ferroceneboronic acid esters.

Synthesis of CT45. To a mixture of 100 mL toluene, 250 mL methanol, and 500 mL water, heated to 50° C., was added 37.9 g (0.18 moles) of copper (II) bromide. A solution of CK71 (13.4 g) in ether was added, and the mixture was stirred vigorously for 30 minutes, maintaining the temperature between 50° C. and 70° C. After 30 minutes, the mixture was cooled to room temperature and extracted with ether. The crude product was concentrated and filtered through a pad of silica gel to produce pure CT45 (6.1 g, 0.10 moles), which contains <1% ferrocene by GC-MS. GC-MS: m/e 266.9 (13), 265.9 (89), 264.9 (15), 263.9 (100), 185.0 (12), 184.0 (74), 136.8 (11), 134.8 (12), 128.1 (69), 127.1 (14), 121.0 (15), 56.0 (36).

Synthesis of CT160. To a solution of 10.7 g (40.5 mmol) of CT45 in 250 mL dry DCM was added 7.0 mL (11.3 g, 60.8 mmol) of 4-bromobutyryl chloride. The solution was cooled to ⁰° C., and 8.1 g (60.8 mmol) of aluminum chloride was added in one portion. The mixture was stirred at 0° C. and monitored by GC-MS. After 25 minutes, the starting material had disappeared, so the reaction was quenched by pouring into 400 mL of ice and 5% aq. NaHCO₃. The pH of the aqueous layer was adjusted to about 7 with 4 M aqueous NaOH, and the DCM layer was removed in a separatory funnel. The aqueous layer was extracted with 2×200 mL 25% ethyl acetate/75% hexanes. The combined organic layers were washed with 100 mL 5% aqueous NaHCO₃ and 100 mL water, dried over Na₂SO₄, filtered, and concentrated. The crude product was filtered through a silica pad and concentrated to yield 16.6 g (40 mmol; 99% yield) of pure CT160. ¹H-NMR (CDCl₃): δ 4.83 (t, 2H), 4.55 (t, 2H), 4.46 (t, 2H), 4.16 (t, 2H), 3.57 (t, 2H), 2.97 (t, 2H), 2.28 (m, 2H). GC-MS: m/e 334.9 (14),333.9 (85), 332.9 (18),331.9 (100),329.9 (15),254.0 (11), 252.0 (11), 167.0 (11), 166.1 (14), 165.1 (23), 152.1 (10), 128.1 (12), 77.1 (10), 69.1 (23), 56.0(12).

Synthesis of SJ6. A solution of 12.0 g (29 mmol) CT160 in 200 mL dry DCM was cooled to ⁰° C. under argon. 29 mL (29 mmol) of a 1.0 M solution of titanium tetrachloride in DCM was added slowly via syringe. Following this addition, 19 mL (116 mmol) of triethylsilane was added slowly via syringe. The ice bath was removed, and the reaction was allowed to proceed overnight at room temperature. After 18 hours, the reaction was complete by TLC, so the reaction was quenched by pouring into 200 mL ice and 5% aqueous NaHCO₃. The DCM layer was separated, and the pH of the aqueous layer was adjusted to >7 with the addition of 4M aqueous NaOH. The aqueous layer was extracted with 2×100 mL hexanes, and the combined organic layers were washed with 100 mL 5% aqueous NaHCO₃ and 100 mL water. The organic layers were dried over Na₂SO₄, filtered, and concentrated to a brown oil. The crude product was purified by flash chromatography to yield 9.2 g (23 mmol; 80% yield) of pure SJ6. ¹H-NMR (CDCl₃): δ 4.31 (t, 2H), 4.13 (t, 2H), 4.07 (t, 2H), 4.05 (t, 2H), 3.42 (t, 2H), 2.39 (t, 2H), 1.90 (m, 2H), 1.67 (m, 2H). GC-MS: m/e 401.9 (47), 400.9 (17), 399.9 (100), 398.9 (10), 397.9 (58), 278.9 (18), 276.9 (19), 240.1 (11), 214.9 (10), 212.9 (11), 175.0 (11), 141.1 (24), 134.9 (11), 91.1 (18).

Synthesis of the above compounds is shown in FIG. 16A.

Synthesis of K158. To a solution of 7.7 g (84 mmol) glycerol in 500 mL anhydrous pyridine was added 50.0 g (147 mmol) of 4,4′-dimethoxytrityl chloride and 0.4 g (4 mol %) N,N-dimethyl4-aminopyridine. The yellow solution was stirred overnight at room temperature. After 16 hours, the pyridine was removed under vacuum, and the residual yellow solid was redissolved in 500 mL dichloromethane. The crude product was extracted twice with 250 mL 5% (w/v) aqueous NaHCO₃, dried over Na₂SO₄, filtered, and concentrated to a yellow foam. The crude product was purified by flash chromatography (with 1% TEA in the eluent) to yield 49.3 g (71 mmol, 84%) pure K158. This could be further purified by recrystallization from hexanes/dichloromethane. ¹H-NMR (DMSO-d₆): δ 7.4 (dd, 4H), 7.1-7.3 (m, 14H), 6.8 (dd, 8H), 4.9 (d, 1H), 3.8 (m, 1H), 3.7 (s, 12H), 3.1 (m, 2H), 3.0 (m, 2H).

Synthesis of SJ7. To a solution of 19.6 g of K158 (28 mmol) in 200 mL dry DMF was added 1. 1 g (28 mmol) of sodium hydride as a 60% dispersion in mineral oil. The suspension was stirred for 1 hour at room temperature, and then a solution of 7.5 g (19 mmol) of SJ6 in 50 mL dry DMF was added dropwise. The suspension was stirred overnight at room temperature. After 15 hours, the reaction was complete by TLC, so the reaction mixture was partitioned between 300 mL water and 300 mL ethyl acetate. The aqueous layer was extracted with 2×300 mL ethyl acetate, and the combined organic layers were washed with 5% aqueous NaHCO₃ and water. The organic layer was then dried over Na₂SO₄, filtered, and concentrated to a brown oil. The crude product was purified by flash chromatography to yield 9.4 g (9.2 mmol; 49%) of pure SJ7. ¹H-NMR (CDCl₃): δ 7.43 (dd, 4H), 7.1-7.3 (m, 14H), 6.8 (dd, 8H), 4.28 (t, 2H), 4.09 (t, 2H), 4.02 (t, 2H), 4.01 (t, 2H), 3.77 (s, 12H), 3.55 (t,1H), 3.4 (m, 4H), 3.1-3.2 (ddd, 2H) 2.3 (m, 2H), 1.5 (m, 4H).

Synthesis of SJ8. To a solution of 12.3 g (12.1 mmol) of SJ7 in 400 mL DCM was added 2.5 g (15 mmol) of trichloroacetic acid. After 15 minutes at room temperature, the reaction was quenched by the addition of 3.5 mL triethylamine in 20 mL methanol. The reaction mixture was extracted with 200 mL 5% aqueous NaHCO₃, dried over Na₂SO₄, filtered, and concentrated. The crude material was purified by flash chromatography to yield 4.5 g (6.3 mmol; 52%) of SJ8 and 5.5 g (5.4 mmol; 45%) recovered SJ7. ¹H-NMR (CDCl₃): δ 7.43 (dd, 2H), 7.1-7.3 (m, 7H), 6.8 (dd, 4H), 4.28 (t, 2H), 4.09 (t, 2H), 4.02 (m, 2H), 4.01 (t, 2H), 3.77 (s, 6H), 3.5 (m,1H), 3.6 (m, 2H), 3.5 (m, 2H), 3.1-3.2 (ddd, 2H), 2.3 (m, 2H), 1.6 (m, 4H).

Synthesis of SJ9. To a solution of 5.0 g (6.8 mmol) of SJ8 in 200 mL anhydrous DCM was added 4.7 mL (3.5 g, 27 mmol) of diisopropylethylamine, and the solution was cooled to 0° C. under argon. To this solution was added 1.8 mL (1.9 g, 8.2 mmol) of N,N-diisopropylamino-cyanoethyl-phosphonamidic chloride via syringe. The ice bath was removed, and the solution was stirred at room temperature. After 1.5 hours, the reaction was complete by TLC. The reaction was diluted with 250 mL DCM and washed with 250 mL 5% aqueous NaHCO₃. The crude product was dried over Na₂SO₄, filtered, and concentrated to a yellow oil. The crude product was purified by flash chromatography and concentrated under vacuum, then dissolved in 5 mL dry ACN and filtered through a 0.45-μ PTFE syringetip filter. The solvent was removed under vacuum, and the pure product was redissolved in anhydrous DCM, transferred to vials, and redried in vacuo. The yield of the reaction was 5.3 g (5.8 mmol; 85% yield). The coupling efficiency of the SJ9 on the DNA synthesizer was 99%. ¹H-NMR (CDCl₃): δ 7.46 (dd, 2H), 7.1-7.3 (m, 7H), 6.8 (d, 4H), 4.28 (t, 2H), 4.11 (t, 2H), 4.06 (m, 2H), 4.03 (t, 2H), 3.79 (s, 6H), 3.5-3.7 (m, 7H), 3.2 (d, 2H), 2.5 (m, 2H), 2.4 (m, 2H), 2.3 (m, 2H), 1.6 (bs, 4H), 1.1 (dd, 12H). ³¹P-NMR (CDCl₃): δ 149.3, 149.2. ES-MS: m/z 937 (M+Na⁺).

Synthesis of the above compounds is shown in FIG. 16B.

Synthesis of CK71. To a pre-cooled solution (−5° C.) of ferrocene (25.1 g, 135 mmol) in dry THF (200 ml) was added 85.0 mL tert-butyllithium in pentane (145 mmol) dropwise over 45 minutes, while the reaction was vigorously stirred. After the addition of tert-butyllithium, the reaction mixture was warmed up to room temperature over a period of 10 minutes. The reaction mixture was then cooled to −78° C., and tributyl borate (40.0 mL, 148.2 mmol) was added dropwise over 45 minutes. The reaction was warmed up to room temperature and stirred for 2 hours, during which time the reaction mixture changed from a slurry to a clear solution. The reaction was quenched by the addition of 100 mL of 5% aqueous HCl. The aqueous layer was separated from the organic layer and extracted with ethyl acetate (2×100 mL). The combined organic layers were then washed with brine, dried over anhydrous sodium sulfate and concentrated, resulting in a red solid. The crude product was purified using pad filtration through silica gel. The sample was loaded as a DCM solution and eluted with hexanes/1% TEA, hexanes/DCM (80/20), and DCM/methanol (97/3). This yielded CK71 (13.5 g) as a yellow solid, which was used for the next reaction without further purification and characterization.

Synthesis of CK73. The crude ferrocenylboronate CK71 (13.5 g) and copper chloride (36.6 g, 214 mmol) were suspended in 500 mL water. The reaction mixture was heated to 65-70° C. and stirred for 4 hours. The reaction was monitored by TLC. When the starting material had been consumed, the mixture was cooled to room temperature, extracted with hexanes (3×150 mL), and dried over anhydrous sodium sulfate. The crude product was purified by silica-gel pad filtration, eluting with hexanes. After removing the solvent, a yellow solid was obtained. GC/MS analysis indicated ˜15% ferrocene was still present, and the product was further purified by partial iodine oxidation.

The column-purified CK73 (7.9 g) was dissolved in 200 mL of hexanes and cooled to 0° C. A solution of iodine (3.42 g) in hexanes was added portionwise, and a dark precipitate (presumably ferrocenium iodide) was observed. The composition of the supernatant was monitored by GC/MS. When the GC/MS indicated the complete consumption of ferrocene, the solution was decanted, filtered through a silica gel pad, and concentrated. After this treatment, 6.8 g of CK73 (30.8 mmol; 23% over two steps) was obtained with 99% purity. GC/MS: m/e 222 (37), 220 (100), 184 (63), 128 (63).

Synthesis of N247. To a solution of CK73 (11.5 g, 52.4 mmol.) in dichloromethane (120 mL), cooled to 0° C., was added bromobutyryl chloride (7.3 mL, 62.8 mmol.) and aluminum chloride (8.4 g, 62.8 mmol). The reaction was stirred at room temperature for 40 minutes, and then quenched by addition of the reaction mixture to 200 mL of cold 5% aqueous NaOH. The mixture was extracted with ethyl ether, and the combined organic layers were extracted with water, dried over sodium sulfate and concentrated. The crude N247 was used in the next reaction without further purification.

Synthesis of N248. To a solution of crude N247 (12.0 g, 26.7 mmol.) in toluene was added powdered zinc (50.0 g, 0.76 mol), mercury chloride (1.5 g, 5.5 mmol) and water (10 mL), followed by 30 mL of concentrated HCl slowly. The mixture was stirred vigorously at room temperature for 2 hours, and then was filtered. The aqueous layer was extracted by hexane three times, and the combined organic layers were washed with water and brine, dried over sodium sulfate, and concentrated. The crude product was purified on a column of 75 g of silica gel packed with hexanes/1% TEA, and eluted with 5-10% ethyl acetate in hexanes to yield the desired product N248 (1.6 g, 74%). GC/MS: m/e 356 (100), 233 (33), 213 (17), 175 (18), 141 (17), 91 (18).

Synthesis of K158. To a solution of 7.79 (84 mmol) glycerol in 500 mL anhydrous pyridine was added 50.0 g (147 mmol) of 4,4′-dimethoxytrityl chloride and 0.4 g (4 mol %) N,N-dimethyl-4-aminopyridine. The yellow solution was stirred overnight at room temperature. After 16 hours, the pyridine was removed under vacuum, and the residual yellow solid was redissolved in 500 mL dichloromethane. The crude product was extracted twice with 250 mL 5% (w/v) aqueous sodium bicarbonate, dried over sodium sulfate, filtered, and concentrated to a yellow foam. The crude product was purified by flash chromatography (with 1% TEA in the eluent) to yield 49.3 g (71 mmol, 84%) pure K158, which could be further purified by recrystallization from hexanes/dichloromethane. ¹H-NMR (DMSO-d₆): δ 7.4 (dd, 4H), 7.1-7.3 (m, 14H), 6.8 (dd, 8H), 4.9 (d, 1H), 3.8 (m, 1H), 3.7 (s, 12H), 3.1 (m, 2H), 3.0 (m, 2H).

Synthesis of SJ59. To a solution of 23.0 g of K158 (33 mmol) in 250 mL dry DMF was added 1.3 g (33 mmol) of sodium hydride as a 60% dispersion in mineral oil. The suspension was stirred for 1 hour at room temperature, and then a solution of 10.6 g (30 mmol) of N248 in 50 mL dry DMF was added dropwise. The suspension was stirred overnight at room temperature. After 15 hours, the reaction was complete by TLC, so the reaction mixture was partitioned between 500 mL water and 500 mL 2:1 (v/v) ethyl acetate/hexanes. The aqueous layer was extracted with 2×300 mL 2:1 (v/v) ethyl acetate/hexanes, and the combined organic layers were dried over sodium sulfate, filtered, and concentrated to a brown oil. The crude product was purified by flash chromatography to yield 9.3 g (9.5 mmol; 31%) of pure SJ59. In addition, 1.8 g (5.0 mmol; 17%) of the unreacted N248 and 2.4 g (8.8 mmol; 30%) of the elimination product SJ60 were also isolated after purification.

¹H-NMR (CDCl₃): δ 7.43 (dd, 4H), 7.1-7.3 (m, 14H), 6.8 (dd, 8H), 4.27 (t, 2H), 4.12 (t, 2H), 4.08 (t, 2H), 3.98 (t, 2H), 3.79 (s, 12H), 3.62 (t, 1H), 3.55 (m, 4H), 3.2-3.3 (ddd, 2H) 2.38 (m, 2H), 1.6 (m, 4H).

Synthesis of SJ61. To a solution of 9.2 g (9.5 mmol) of SJ59 in 300 mL DCM was added 1.5 g (9.5 mmol) of trichloroacetic acid. After 30 minutes at room temperature, the mixture was quenched by the addition of 5 mL triethylamine in 20 mL methanol. The reaction mixture was extracted with 300 mL 5% aqueous sodium bicarbonate, dried over sodium sulfate, filtered, and concentrated. The crude material was purified by flash chromatography to yield 3.1 g (4.6 mmol; 49%) of SJ61 and 4.6 g (4.8 mmol; 50%) recovered SJ59. ¹H-NMR (DMSO-d₆): δ 7.48 (dd, 2H), 7.2-7.4 (m, 7H), 6.9 (dd, 4H), 4.6 (t,1H), 4.44 (t, 2H), 4.2 (t, 2H), 4.17 (m, 2H), 4.14 (t, 2H), 3.81 (s, 6H), 3.6 (m, 1H), 3.48 (m, 2H), 3.38 (m, 2H), 3.1-3.2 (ddd, 2H), 2.4 (m, 2H), 1.6 (m, 4H). Synthesis of SJ63. To a solution of 3.4 g (5.0 mmol) of SJ61 in 100 mL anhydrous DCM was added 3.5 mL (2.6 g, 20 mmol) of N,N-diisopropylethylamine and 60 mg (0.5 mmol) of N,N-dimethylaminopyridine, and the solution was cooled to 0° C. under argon. To this solution was added 1.4 mL (1.4 g, 6.0 mmol) of N,N-diisopropylamino-cyanoethyl-phosphonamidic chloride via syringe. The ice bath was removed, and the solution was stirred at room temperature. The reaction was monitored by TLC. After 2 hours, the mixture was diluted with 100 mL DCM and washed with 100 mL 5% aqueous sodium bicarbonate. The DCM solution was dried over sodium sulfate, filtered, and concentrated. The crude product was purified by flash chromatography and concentrated under vacuum, to give the desired product SJ63 as a yellow oil (3.7 g; 4.4 mmol; 87%).

The purified SJ63 was then dissolved in 10 mL dry ACN and filtered through a 0.45-μ PTFE syringetip filter. The solvent was removed under vacuum, and the pure product was redissolved in anhydrous DCM, transferred to vials, and redried in vacuo. The coupling efficiency of the SJ63 on the DNA synthesizer was 99.8%. ¹H-NMR (DMSO-d₆): δ 7.38 (dd, 2H), 7.1-7.3 (m, 7H), 6.8 (d, 4H), 4.34 (t, 2H), 4.10 (t, 2H), 4.07 (m, 2H), 4.04 (t, 2H), 3.71 (s, 6H), 3.4-3.7 (m, 7H), 3.3 (m, 2H), 3.0 (m, 2H), 2.6-2.7 (dt, 2H), 2.3 (m, 2H), 1.5 (bs, 4H), 1.0-1.1 (m, 12H1). ³¹P-NMR (DMSO-d₆): δ 148.3, 148.2. ES-MS: m/z calculated for C₄₇H₁₅₈CFeN₂O₆P, 868; found, 868 (M+H⁺) and 891 (M+Na⁺).

FIG. 16C depicts the synthesis of the above compounds.

Non-Nucleosidic Ferrocene Phosphoramidite

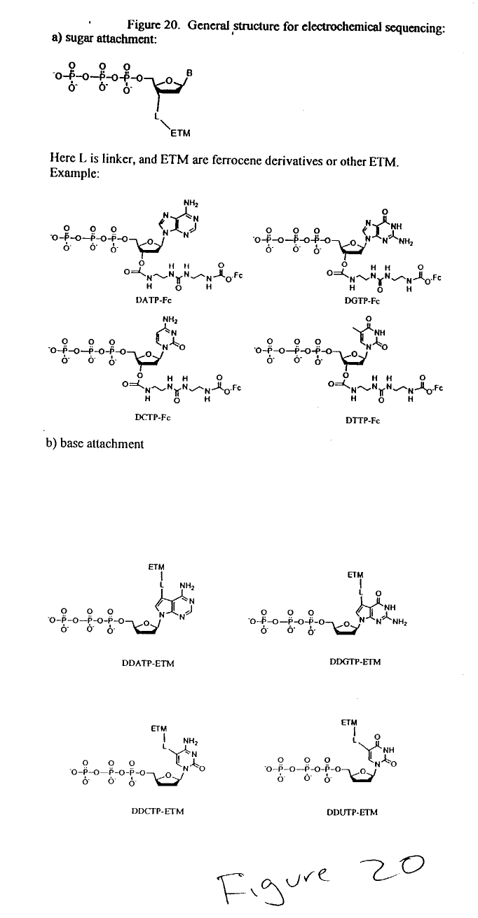

Synthesis of non-nucleosidic ferrocene phosphoramidites is depicted in FIGS. 17A and B.

FIG. 17A depicts the synthesis of the following compounds:

Synthesis of N1. To a solution of ferrocene (41.50 g, 223.10 mmol) in dry dichloromethane (750 ml) 4-bromobutyryl chloride (26.00 mL, 224.59 mmol) was added at room temperature and then cooled to 0° C. Aluminum chloride (32.00 g, 234.00 mmol) was added to the above solution under argon at 0° C., while allowing the reaction stirring. The reaction was warmed up to room temperature and monitored with TLC until no ferrocene left (about 1.5 h). The reaction mixture was slowly poured into ice water and separated, followed by extracting the aqueous layer with dichloromethane (3×200 mL). The combined organic layer was then washed with water (400 mL), 5% NaHCO₃ and dried over anhydrous sodium sulfate. After removal of the solvent, the residue was further dried under high vacuum for 2 to 3 hours, giving N1 as a deep orange-colored oil (73.96 g, 99%), which was not further purified and used for next step reaction (GC/MS showed pure compound). Anal. Calcd. for (C₁₄H₁₅BrFeO): 335, Found (GC-MS) 255 (18, M-Br) 254 (100), 186 (27), 121(27), 56 (18).

Synthesis of N2. To a slurry of zinc powder (398 g, 4710 mmol) in water (500 ml) mercury chloride (10.36 g, 38.16 mmol) was added and mixed thoroughly. After the solids settled, the water was decanted off. N1 (73.96 g, 220.78 mmol) was dissolved in toluene (1800 mL) and transferred to the above Zn(Hg) container. Concentrated HCl (440 mL) was added by portion while the mixture was stirred vigorously using a mechanical stirrer. The reaction was monitored with TLC until no N1 left (1 to 2 h). The reaction mixture was then filtered through a 2-inch bed of Celite® and washed with hexane. After separating the aqueous layer, the organic layer was washed with water (500 mL), 5% sodium bicarbonate (500 mL), and water (500 mL), and dried over anhydrous sodium sulfate. After removing solvents, the crude product was purified by column chromatography on silica gel using hexane as the elution solvent. Only one portion was collected and concentrated to give a orange oil of N2 (52.25 g, 74%), which can be stored at 0-4° C. without decomposing. The structure was confirmed by GC/MS.

Anal. Calcd. for (C₁₄H₁₇BrFe): 321, Found (GC-MS) m/z 321(15) 320 (100), 199 (80), 121(50).

Synthesis of K158. To a solution of glycerol (7.7 g, 84 mmol) in 500 mL anhydrous pyridine was added dimethoxytrityl chloride (50 g, 147 mmol) and N,N-dimethyl-4-aminopyridine (0.4 g, 4 mol %). The yellow solution was stirred overnight at room temperature. After 16 hours, the pyridine was removed under vacuum, and the residual yellow solid was redissolved in 500 mL dichloromethane. The crude product was extracted twice with 250 mL 5% (w/v) aqueous NaHCO₃, dried over Na₂SO₄, filtered, and concentrated to a yellow foam. The crude product was purified by flash chromatography (with 1% TEA in the eluent) to yield 49 g (71 mmol, 84%) pure K158. This could be further purified by recrystallization from hexanes/dichloromethane. ¹H-NMR (300 M Hz, DMSO-d₆): δ 7.4 (dd, 4H), 7.1-7.3 (m, 14H), 6.8 (dd, 8H), 4.9 (d, 1H), 3.8 (m, 1H), 3.7 (s, 12H), 3.1 (m, 2H), 3.0 (m, 2H).

Synthesis of K159. To a solution of K158 (33.53 g, 48.10 mmol) in DMF (300 mL) sodium hydride was added in one portion and the reaction was stirred for 1 hour under argon. A solution of N2 (11.31 g, 35.23 mmol) in DMF (50 mL) was then added to the reaction flask and the reaction was stirred overnight at room temperature under argon. TLC indicated the consumption of N2. The reaction was quenched with water (200 mL) and extracted with ethyl acetate/hexane (300/100, 75/25, 75/25) until the organic layer become colorless. The combined organic layer was then dried over anhydrous sodium sulfate and the solvent was removed under reduced pressure. The remaining DMF was further co-evaporated with ethanol, resulting in a orange residue of crude product (53.52 g). The crude product was purified with column chromatography on silica gel. The column was packed with 1% triethylamine/hexane and eluted with 5%, 10%, 20% ethyl acetate/hexane, 1:1 ethyl acetate/hexane, 2:1 ethyl acetate/hexane (all contain 1% of triethylamine). The first fraction was the elimination product. The second fraction was the desired product of K159 which was collected and concentrated under vacuum to afford a orange color oil (15.62 g, 35%). The third fraction was the recovered K158.

Synthesis of K160. To a solution of K159 (11.79 g, 12.61 mmol) in dichloromethane (200 mL) trichloroacetic acid (2.50 g, 15.29 mmol) in dichloromethane (100 mL) was added and the reaction was stirred for 30 minutes. A solution of triethylamine (2.31 mL, 16.45 mmol) in methanol (10 mL) was added to the reaction flask and stirred for 5 more minutes. The reaction mixture was then washed with 5% sodium bicarbonate (2×200 mL) and dried over sodium sulfate. After removal of the solvents, the crude product was purified by column chromatography on silica gel. The column was packed with 1% triethylamine/hexane and eluted with 10%, 20% ethyl acetate/hexane (all contain 1% of triethylamine). The first fraction was the recovered starting material K159, while the second fraction was the desired product of K160. The recovered starting material of K159 was used to repeat the reaction over again and more K160 was produced. The combined K160 was put on the high vacuum line to remove all remaining solvents, resulting in a orange oil (5.04 g, 64% combined yield based on the consumed starting material).

Synthesis of K161. To a pre-cooled solution of K160 (5.04 g, 7.97 mmol) in dichloromethane (100 ml) C96 (1.36 g, 7.97 mmol) was added at 0° C., followed by addition of 2-cyanoethyl N,N,N′,N′-tetraisopropyl phosphane (5.25 mL, 15.94 mmol). The reaction was warmed up to room temperature and stirred for 2 hours. TLC was used to monitor the reaction util all of the starting material of K160 was consumed. The reaction mixture was washed with di-water and the water layer was in turn extracted with dichloromethane (2×10 mL). The combined organic layer was dried over sodium sulfate and the solvent was removed. Purification was performed by column chromatography on silica gel, eluting with 0-30% ethyl acetate/hexane (1% triethylamine), to afford the final product of K161 (4.67 g, 72%). Coupling efficiency: 98%. ¹H NMR (300 MHz, CDCl₃): δ 7.35-7.25 (m, 9H, Ph), 6.81 (d, J=8.7 Hz, 4H, Ph-OMe), 4.07 (s, 5H, Fc), 4.02 (m, 4H, Fc), 3.78 (s, 6H, 2 MeO), 3.77-3.50 (m, 11H), 3.15 (d, 2H), 2.50 (m, 2H), 2.35 (t, 2H), 1.58 (m, 2H), 1.17-1.08 (m, 12H, 2 (CH₃)₂CH). Anal. calcd. for (C₄₇H₅₉FeN₂O₆P): 834, Found: MS 834 (M+), 857 (MNa⁺).

FIG. 17B depicts the synthesis of the following compounds:

Synthesis of N200. 1 equiv. of EK5 was reacted with 2.5 equiv. of nBuLi in CH₂Cl₂at −78° C. Then the temperature was warmed up to room temperature and 2.1 equiv. of FeCl₂ was added. The reaction was stirred overnight. Saturated NaHCO3 was added to quench the reaction. Filtration followed by extracting the filtrate with CH₂Cl₂ provided an orange oily residue. After column separation, N200 was obtained in 60% yield.

Synthesis of N203. 1 eqiv. of N200 was reacted with 0.6 equiv. of DMTCl in the presence of TEA (1.2 eqiv.) in CH₂Cl₂ for 3 hours. TLC was used to monitor the disappearance of DMTCI, indicating the finishing of the reaction. The yield of N₂O₃ is about 65% based on the consumed starting material.

Synthesis of N204. 1 equiv. of N₂₀₃ was reacted with 1.5 equiv. of Bis-(N,N)-diisopropylamino)-2-cyanoethoxyphosphine in the presence of C96 (0.7 equiv.) in CH₂Cl₂ for 3 hours. After column separation, 85% of N₂O₄ was obtained. Anal. calcd. for (C₄₆H₅₆FeN₂O₅P): 803, Found: MS 803 (M⁺), 826 (Mna⁺).

Ferrocenes With High Redox Potentials

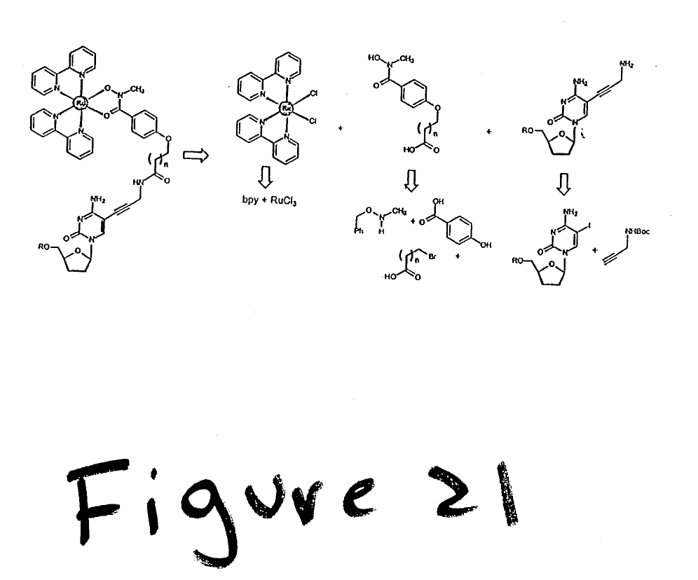

FIGS. 18A-E depict the synthesis of ferrocenes with high redox potentials.

FIG. 18A depicts the synthesis of the following compounds:

Synthesis of CT184. To a solution of ferrocene (40.0 g, 215 mmol) in dry ether (500 ml) was added tetramethyl ethylenediamine (71 ml, 55.0 g, 473 mmol) and nBuLi (1.6 M in hexane, 298 ml, 473 mmol) at room temperature. The mixture was stirred for 16 hours at room temperature and cooled to −78° C., and then toluenesulfonyl chloride (102.5 g, 537.5 mmol) was added. The mixture was stirred for 1 h at the −78° C., warmed to room temperature and stirred for an additional hour. The mixture was diluted with 300 ml hexane then was washed with water, brine, dried (Na₂SO₄) and concentrated. The residue was passed a silica gel column eluted with hexane and dichloromethane (90/10). After removed solvents, 45 g yellow solid was obtained (GS/MS showed with 80% desired product). The solid was recrystallized from hexane one time to afford CT184 (21 g, with purity larger than 99% from GC/MS) as a light orange color crystal. GC/MS: m/e 256 (48), 254 (78), 128 (100), 127 (27), 102 (13).

Synthesis of CT185. To a solution of CT184 (5.00 g, 19.61 mmol) and 4-bromobutyryl chloride (4.0 g, 2.5 ml, 21.58 mmol) in dry dichloromethane (100 ml) was added aluminum chloride (2.88 g, 21.58 mmol) at 0° C. After addition of starting materials, the cooling bath was removed and mixture was stirred for another 30 min. TLC showed the reaction complete. The mixture was poured into ice water and extracted with hexane. The organic layers were washed with water, 5% NaHCO₃, and brine, dried (Na₂SO₄) and concentrated. The residue was purified with silica gel chromatography eluted with hexane, 10% ethyl acetate/hexane, to give the desired product CT185 as a reddish oil (7.60 g, 95%) with a ˜4:5 mixture of the α and β isomers. GC/MS: m/e for isomer I (retention time 13.618 min): 324 (59), 322 (100), 165 (47), 155 (95); for isomer 11 (retention time 13.687 min): 324 (66), 322 (100), 195 (35), 167 (24), 65 (62), 102 (23).

Synthesis of CT186. To a suspending solution of zinc (11.60 g, 178.20 mmol) in dry THF (150 ml) was added diiodomethane (7.2 ml, 23.9 g, 89.13 mmol) at room temperature. After stirred for 30 min, the dark and thick mixture was cooled to 0° C., then titanium tetrachloride (18.0 mL, 1.0 M/CH₂Cl₂, 17.82 mmol) was added dropwise. The dark green black mixture was further stirred for 30 min at room temperature. To the mixture was added CT185 (7.20 g, 17.82 mmol) in dry THF (35 mL) dropwise. The mixture was stirred for 7 hours at room temperature. To the reaction mixture was added a mixture of hexane/ether (300 mL, 9:1). Then the mixture was washed with 5% NaHCO₃ and brine, dried (Na₂SO₄) and concentrated. The residue was purified with silica gel chromatography (packed with 2% TEA/hexane) eluted with hexane, and 10% dichloromethane/hexane to give the desired product CT186 (3.65 g, 65% based on the consumed starting material CT185, 1:3 α:β regional isomer) and recovered starting material CT185 (1.60 g, 22%). GC/MS: m/e for CT186: for isomer α (retention time 15.385 min): 404 (45), 402 (100), 400 (66), 320 (18), 178 (26), 165 (41), 152 (34), 129 (40), 115 (43); for isomer β (retention time 15.413 min): 404 (45), 402 (100), 400 (68), 320 (15), 178 (18), 165 (29), 152 (23), 129 (26), 115 (29), 91 (28).

Synthesis of CT187. To a solution of CT186 (60 mg, 0.14 mmol) in dioxane (2 mL) and methanol (2 mL) was added sodium sulfite (220 mg, 2.82 mmol) in water (3 mL). The mixture was stirred at 70° C. for overnight. The starting material disappeared and a single spot formed on TLC (10% MeOH/CH₂Cl₂). After cooled to room temperature, solid was filtered. After removal organic solvent, a yellow aqueous solution was obtained, which was used for the gold ball experiment without further purification.

Synthesis of SJ30. To a solution of 1-(4-bromobutyryl)-1,1′-dichloroferrocene (5.40 g 13.4 mmol) in toluene (200 mL) was added Zn powder (80.00 g), HgCl₂ (8.00 g), deionized water (115 mL), and concentrated hydrochloric acid (115 mL). The 3-phase mixture was stirred vigorously at room temperature to prevent the metals from aggregating. After 2 hours, the liquid was decanted, and the metal amalgam was washed with 50 mL hexanes four times. The toluene solution was separated from the aqueous layer and combined with the hexane washings, dried (Na₂SO₄), filtered, and concentrated to afford brown oil. The crude product was purified by flash chromatography to yield 4.10 g (10.5 mmol, 79%) of the pure product SJ30 as a ˜2:1 mixture of the α and β isomers. GC-MS: m/e (for major isomer) 392 (45), 390 (100), 388 (65), 310 (10), 308 (11), 269 (14), 267 (22), 155 (17), 141 (32), 117 (28), 115 (45), 91 (46).

Synthesis of K158. To a solution of glycerol (7.7 g, 84 mmol) in 500 mL anhydrous pyridine was added dimethoxytrityl chloride (50 g, 147 mmol) and N,N-dimethyl-4-aminopyridine (0.4 g, 4 mol %). The yellow solution was stirred overnight at room temperature. After 16 hours, the pyridine was removed under vacuum, and the residual yellow solid was redissolved in 500 mL dichloromethane. The crude product was extracted twice with 250 mL 5% (w/v) aqueous NaHCO₃, dried over Na₂SO₄, filtered, and concentrated to a yellow foam. The crude product was purified by flash chromatography (with 1% TEA in the eluent) to yield 49.0 g (71 mmol, 84%) pure K158. This could be further purified by recrystallization from hexanes/dichloromethane. ¹H-NMR (300 M Hz, DMSO-d₆): δ 7.4 (dd, 4H), 7.1-7.3 (m, 14H), 6.8 (dd, 8H), 4.9 (d, 1H), 3.8 (m, 1H), 3.7 (s, 12H), 3.1 (m, 2H), 3.0 (m, 2H).

Synthesis of SJ34. To a solution of K158 (25.7 g, 37 mmol) in 200 mL anhydrous DMF was added a 60% dispersion of NaH in mineral oil (1.5 g, 37 mmol). The suspension was stirred for 1 hour at room temperature, and a solution of SJ30 (7.2 g, 18.5 mmol) in 100 mL was added via syringe The suspension was stirred overnight at room temperature. After 21 hours, the reaction was quenched by the addition of 300 mL 2.5% (w/v) aqueous NaHCO₃, and extracted twice with 300 mL 2:1 (v/v) ethyl acetate-hexanes. The combined organic layers were washed with 100 mL water, dried over Na₂SO₄, filtered, and concentrated to a dark brown oil. The crude SJ34 was purified by flash chromatography (with 1% TEA in the eluent) to yield 10.5 g (10.4 mmol, 56% yield) of the desired product, as a ˜2:1 mixture of the a and b isomers. ¹H-NMR (300 MHz, CDCl₃): d 7.4 (dd, 4H), 7.1-7.3 (m, 14H), 6.7 (dd, 8H), 4.3 (m, 3H), 3.94.1 (m, 4H), 3.8 (s, 12H), 3.5 (m, 2H), 3.3 (m, 2H), 2.5 (m, 2H), 2.2 (m, 2H), 1.5 (m, 4H).

Synthesis of SJ40. To a solution of SJ34 (9.7 g, 9.7 mmol) in 200 mL dichloromethane was added a solution of trichloroacetic acid (1.7 g, 10 mmol) in 100 mL dichloromethane. After stirring for 15 minutes at room temperature, the reaction was quenched by the addition of a solution of triethylamine (1.6 mL, 1.2 g, 12 mmol) in 20 mL methanol. After stirring for 5 minutes at room temperature, the mixture was extracted twice with 200 mL 5% (w/v) aqueous NaHCO₃, dried over Na₂SO₄, filtered, and concentrated to give a brown oil. The crude product was purified by flash chromatography (with 1% TEA in the eluent) to yield 1.39 (1.9 mmol, 20% yield) of the desired product SJ40 and 6.89 (6.8 mmol, 70%) of the recovered starting material SJ34. ¹H-NMR (300 MHz, DMSO-d₆): d 7.4 (dd, 2H), 7.1-7.3 (m, 7H), 6.9 (dd, 4H), 4.4 (m, 3H), 414.2 (m, 4H), 3.7 (s, 6H), 3.5 (bm, 2H), 3.4 (m, 2H), 2.4 (m, 2H), 2.2 (m, 2H), 1.5 (m, 4H).

Synthesis of SJ42. To a solution of SJ40 (1.33 g, 1.9 mmol) in 60 mL dry dichloromethane was added N,N-dimethyl-4-aminopyridine (10 mg, 4 mol %) and diisopropylethylamine (1.3 mL, 0.98 g, 7.6 mmol). The yellow solution was cooled to 0° C. in an ice-water bath, and 2-cyanoethyl diisopropylchlorophosphoramidite (0.51 mL, 0.54 g, 2.3 mmol) was added with stirring via syringe. The ice bath was removed, and the solution was allowed to warm to room temperature. After 2 hours, the reaction mixture was diluted with 100 mL dichloromethane and extracted with 50 mL 5% (w/v) aqueous NaHCO₃ and 50 mL water. The dichloromethane layer was dried over Na₂SO₄, filtered, and concentrated to give a brown oil. The crude product was purified by flash chromatography to yield 1.4 g (1.6 mmol, 82%) of the desired product SJ42. The pure phosphoramidite was dissolved in 5 mL dry acetonitrile, filtered through a 0.45-m PTFE syringe-tip filter, and dried in vacuo. The yellow oil was then redissolved in 7 mL anhydrous dichloromethane, and aliquots were transferred to DNA-synthesizer vials and redried in vacuo overnight. ¹H-NMR (300 MHz, DMSO-d₆): d 7.4 (dd, 2H), 7.1-7.3 (m, 7H), 6.9 (dd, 4H), 4.4 (m, 3H), 4.14.2 (m, 4H), 3.7 (s, 6H), 3.4-3.7 (bm, 6H), 3.1 (m, 2H), 2.7 (m, 4H), 2.4 (m, 2H), 2.2 (m, 2H), 1.6 (m, 4H), 1.1 (m, 12H). Anal. Calcd. for C₄₇H₁₇FeN₂O₆P: 904. Found: 904 and 927 (M+Na⁺).

FIG. 18B depicts the synthesis of the following compounds:

Synthesis of N225. To a solution of toluenesulfinic acid (175.0 g, 0.98 mol.) in water (600 mL) slowly added bromine in cold methanol until the orange color persisted. More toluenesulfinic acid solution was added to change the color from orange to slightly yellow. The precipitate was filtered, washed by water. The solid was passed through a short silica gel column with dichloromethane. The crude product was purified on a column of 300 g of silica gel eluted by dichloromethane to yield 134.6 g of N225 (69%). ¹H NMR (300 MHz, CDCl₃) 7.87 (d, 2H), 7.30 (d, 2H), 2.49 (s, 3H).

Synthesis of K164. To a solution of ferrocene (30.00 g, 0.16 mol.) in ethyl ether (1 L) was added n-butyl lithium (220 mL, 1.6 M in hexane) and tetramethylethylenediamine (27.0 mL, 0.18 mol.). The solution was purged by argon for 10 min., and then was stirred at room temperature overnight. The mixture was cooled to −78° C., and N225 (90.0 g, 0.38 mol.) was added. The reaction mixture was maintained at this temperature for 1 hour, then slowly warmed up to room temperature, and was stirred an additional 30 min. before being quenched by 30 mL of water. The mixture was filtered, and the solid was extracted by hexane several times. The combined organic layers were extracted by water, dried over sodium sulfate, and concentrated. The crude product was purified on a column of 400 g of silica gel eluted by hexane to provide the desired product K164 (40.0 g, 72%). The product could be further purified by recrystallization from methanol. GC/MS: m/e 346 (30), 344 (63), 342 (36), 128 (100), 102 (13).

Synthesis of CT176. To a solution of K164 (3.44 g, 10.12 mmol) and 4-bromobutyryl chloride (2.79 g, 1.7 ml, 15.00 mmol) in dry dichloromethane (70 ml) was added aluminum chloride (2.00 g, 15.00 mmol) at 0° C. After addition of starting materials, the cooling bath was removed and mixture was stirred for another 30 min. TLC showed the reaction complete. The mixture was poured into ice water and extracted with hexane/ether. The organic layers were washed with water, 5% NaHCO₃, and brine, dried (Na₂SO₄) and concentrated. The residue was purified with silica gel chromatography eluted with hexane, 10% ethyl acetate/hexane, to give the desired product CTI76 as a reddish oil (4.30 g, 87%) with a 2:5 mixture of the a and b isomers. GC/MS: m/e for isomer a (retention time 14.733 min): 414 (29), 412 (64), 410 (40), 195 (20), 165 (40), 155 (100); for isomer b (retention time 14.767 min): 414 (47), 412 (100), 410 (61), 195 (51), 165 (63), 153 (31), 152 (29), 139 (23), 102 (26).

Synthesis of N221. To a solution of 1-(4-bromobutyryl)-1,1′-dibromoferrocene (1.00 g 2.04 mmol) in toluene (75 mL) was added Zn powder (15.00 g), HgCl₂ (1.50 g), deionized water (30 mL), and concentrated hydrochloric acid (30 mL). The 3-phase mixture was stirred vigorously at room temperature to prevent the metals from aggregating. After 1.5 hours, the liquid was decanted, and the metal amalgam was washed with 50 mL hexanes four times. The combined organic layers were washed with water, 5% NaHCO₃, dried (Na₂SO₄) and concentrated. The crude product was purified by flash chromatography to yield N221 (830 mg, 85%) as yellow oil with 1:2 mixture of the a and b regional isomers. GC/MS: m/e for isomer a (retention time 15.939 min): 480 (91), 478 (100), 476 (39), 141 (89), 115 (90), 91 (65), 77 (46); for isomer b (retention time 16.070 min): 482 (29), 480 (90), 478 (100), 476 (39), 141 (26), 115 (19).

With N221 in hand, the preparation of phosphoramidite with dibromo functionality will be easily realized according to the similar procedures in Scheme 8.

FIG. 18C depicts the synthesis of the following compounds:

Synthesis of CT151. To a suspension solution of ferrocene carboxylic acid (1.00 g, 4.35 mmol) in dichloromethane (20 mL) was added N-hydroxysuccinimide (1.00 g, 8.69 mmol) and 1,3-dicyclohexylcarbodiimide (1.79 g, 8.69 mmol). The mixture was stirred for 3 hours at room temperature. To the mixture was added 3-aminopropanol (1.63 g, 1.67 mL, 21.75 mmol) in dichloromethane (20 mL). Then the mixture was further stirred for an additional 3 hours. The mixture was concentrated at reduced pressure, purified on silica gel column eluted with ethyl acetate to provide the desired product CT151 (0.92 g, 74%). Anal. Calcd. (for C₁₄H₁₇FeNO₂) 287. Found 287.

Synthesis of CT171. To a solution of CT151 (0.95 g, 3.31 mmol) in dichloromethane (30 mL) was added C96 (566 mg, 3.31 mmol). The mixture was cooled to 0° C., and N,N,N′,N′-tetraisopropylamino, 2-cyanoethoxy phosphane (3.2 mL, 2.98 g, 9.93 mmol) was added. The reaction mixture was warmed up to room temperature and stirred for 3 hours at room temperature. The mixture was diluted in 100 mL of dichloromethane, extracted by waster three times, dried over sodium sulfate and concentrated. The crude product was purified on a silica gel column packed with 1% TEA in hexane, and eluted with 1%TEA & 10-30% ethyl acetate in hexane to yield the desired product CTI71 as a yellow sticky oil (0.94 g, 58%). Anal. Calcd. for C₂₃H₃₄FeN₃,₃P: 487.35. Found: 487.

FIG. 18D depicts the synthesis of the following compounds:

Synthesis of CT186. To a suspending solution of zinc (11.60 g, 178.20 mmol) in dry THF (150 ml) was added diiodomethane (7.2 ml, 23.9 g, 89.13 mmol) at room temperature. After stirred for 30 min, the dark and thick mixture was cooled to ⁰° C., then titanium tetrachloride (18.0 mL, 1.0 M/CH₂Cl₂, 17.82 mmol) was added dropwise. The dark green black mixture was further stirred for 30 min at room temperature. To the mixture was added CT185 (7.20 g, 17.82 mmol) in dry THF (35 mL) dropwise. The mixture was stirred for 7 hours at room temperature. To the reaction mixture was added a mixture of hexane/ether (300 mL, 9:1). Then the mixture was washed with 5% NaHCO₃ and brine, dried (Na₂SO₄) and concentrated. The residue was purified with silica gel chromatography (packed with 2% TEA/hexane) eluted with hexane, and 10% dichlormethane/hexane to give the desired product CT186 (3.65 g, 65% based on the consumed starting material CT185, 1:3 α:β regional isomer) and recovered starting material CT185 (1.60 g, 22%). GC/MS: m/e for CT186: for isomer α (retention time 15.385 min): 404 (45), 402 (100), 400 (66), 320 (18), 178 (26), 165 (41), 152 (34), 129 (40), 115 (43); for isomer β (retention time 15.413 min): 404 (45), 402 (100), 400 (68), 320 (15), 178 (18), 165 (29), 152 (23), 129 (26), 115 (29), 91 (28). With CT186 in hand, the preparation of phosphoramidite of alkenyl dichloro ferrocene will be easily carried out according to the procedures in Scheme 8.

FIG. 18E depicts the synthesis of the following compounds:

Synthesis of SJ21. To a solution of SJ18 (1.15 g, 1.79 mmol) in dichloromethane (20 mL) was added C96 (620 mg, 3.60 mmol). The mixture was cooled to 0° C., and N,N,N′,N′-tetraisopropylamino, 2-cyanoethoxy phosphane (1.48 mL, 1.36 g, 4.50 mmol) was added. The reaction mixture was warmed up to room temperature and stirred for 2 hours at room temperature. The mixture was diluted in 60 mL of dichloromethane, extracted by waster three times. The organic layers were dried over sodium sulfate and concentrated. The crude product was purified on a silica gel column packed with 1% TEA in hexane, and eluted with 1%TEA & 5-15% ethyl acetate in hexane to yield the desired product SJ21 as a yellow oil (1.27 g, 86%). Anal. Calcd. for C₄₈H₅₇FeN₂O₆P: 844.33. Found: 844.

Ferrocene Derivatives for Post-Synthesis of Nucleic Acid Probes

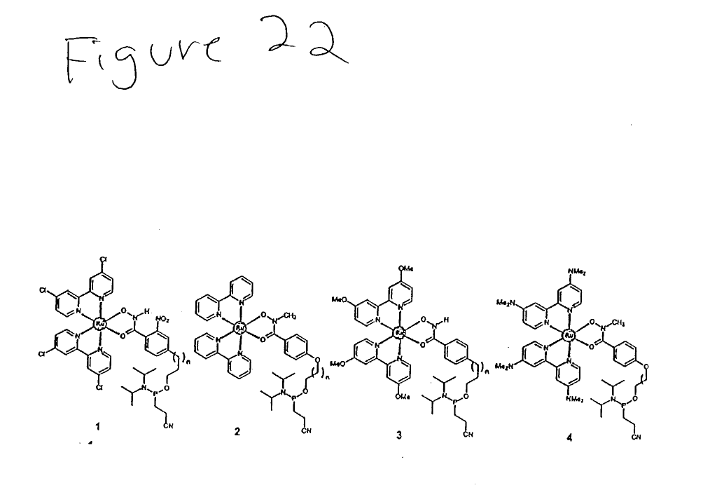

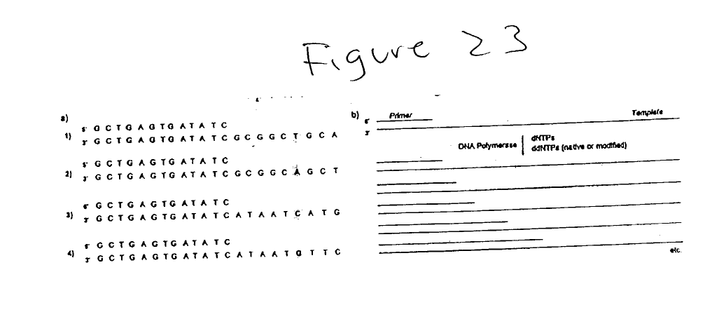

FIG. 19 depicts one means for the post synthesis of nucleic acid probes comprising ferrocene.

Synthesis of N235. To a solution of N219 (0.50 g, 1.3 mmol.) in N,N-dimethylformamide (DMF, 10 mL) was added potassium acetate (0.64 g, 6.6 mmol.), and the reaction was heated at 75° C. for 2 hours. The mixture was cooled to room temperature, and was diluted in 120 mL of ethyl ether. The organic layer was extracted by water, dried over sodium sulfate, and concentrated. The crude product was dissolved in 5 mL of 1,4-dioxane and 1 mL of methanol. To the solution was added 1.6 mL of NaOH solution (4.0 M), and the mixture was stirred at room temperature for 30 minutes. After normal work-up, the crude was purified on a column of 25 g of silica gel. The column was packed in 1% TEA in hexane, and was eluted by 10-50% ethyl acetate in hexane to yield the desired product (0.42 g, 88%).

Synthesis of N241. To a solution of N235 (0.5 g, 1.6 mmol.) in DMF (10 mL) was added NaH (60% on mineral oil, 130 mg, 3.2 mmol.), and the mixture was stirred at room temperature for 10 minutes. A solution of disuccinimidyl carbonate (0.6 g, 2.4 mmol.) in DMF (10 mL) was added to the reaction. The reaction was maintained at room temperature overnight. The mixture was concentrated, and was diluted in ethyl ether. The organic layer was extracted by water, dried over sodium sulfate and concentrated. The crude product was purified on a quick column of 25 g of silica gel. The column was packed in 1% TEA in dichloromethane (DCM) and was eluted by DCM to yield the desired product. The fractions were concentrated, and co-evaporated in acetonitrile to remove TEA and yield the desired product (0.36 g, 50%). ¹H NMR (300 MHZ, CDCl₃) 4.31 (t, 2H), 4.03 (broad, 2H), 3.80 (broad, 1H), 3.64 (broad, 4H), 2.95 (s, 3H), 2.87 (s, 3H), 2.83 (s, 4H), 2.26 (m, 2H), 1.74 (m, 2H), 1.56 (m, 2H); MS C₂₁H₂₅FeNO₇ expected 459, found 460 (MH⁺).

Synthesis of CT193. To a solution of N2 (4.50 g, 14.00 mmol.) in N,N-dimethylformamide (DMF, 80 mL) was added potassium acetate (4.14 g, 42.20 mmol.), and the reaction was heated at 80° C. for 1 hours. There was no starting material left monitored with TLC (CH₂Cl₂/hexane (25/75)). The mixture was diluted with hexane/dichloromethane (7/3) and washing with brine, dried (NaSO₄) and concentrated to give the desired product. Both TLC and GC/MS indicated the formation of the pure product CT195. The product was used for the next step reaction without further purification. GC/MS: m/e 310 (20), 300 (100), 199 (28), 175 (26), 121 (31).

Synthesis of CT194. To a solution of CT195, prepared as indicated as above, in 40 mL of 1,4-dioxane and 8 mL of methanol was added 4.5 mL of NaOH solution (4.0 M, 18.20 mmol), and the mixture was stirred at room temperature for 30 minutes. The mixture was diluted with hexane/dichloromethane (7/3), and washed with brine, dried (NaSO₄) and concentrated. The crude product was purified on a silica gel column (packed with 1%TEA/hexance) eluted by 10-30% ethyl acetate in hexane to yield the desired product as yellow oil (3.26 g, 90% for the two steps). GC/MS: m/e 258 (100), 199 (44), 172 (27), 121 (46).

Synthesis of N238. To a solution of CT194 (0.5 g, 1.9 mmol.) in DMF (10 mL) was added NaH (60% on mineral oil, 140 mg, 3.4 mmol.), and the mixture was stirred at room temperature for 10 minutes. A solution of disuccinimidyl carbonate (0.6 g, 2.4 mmol.) in DMF (10 mL) was added to the reaction. The reaction was maintained at room temperature overnight. The mixture was concentrated, and was diluted in ethyl ether. The organic layer was extracted by water, dried over sodium sulfate and concentrated. The crude product was purified on a quick column of 25 g of silica gel. The column was packed in 1% TEA in dichloromethane (DCM) and was eluted by DCM to yield the desired product. The fractions were concentrated, and co-evaporated in acetonitrile to remove TEA and yield the desired product (0.40 g, 50%). ¹H NMR (300 MHZ, CDCl₃) 4.30 (t, 2H), 4.10 (broad, 9H), 2.92 (s, 4H), 2.36 (m, 2H), 1.78 (m, 2H), 1.61 (m, 2H); MS C₁₉H₂₁FeNO₅ expected 399, found 399 (M+).

Synthesis of CT195. To a solution of CT186 (0.45 g, 1.14 mmol.) in N,N-dimethylformamide (DMF, 10 mL) was added potassium acetate (0.56 g, 5.72 mmol.), and the reaction was heated at 60° C. for 1 hours. There was no starting material left monitored with TLC (CH₂Cl₂/hexane (25/75)). The mixture was diluted with hexane/dichloromethane (7/3) and washing with brine, dried (NaSO₄) and concentrated to give the desired product (0.45 g). Both TLC and GC/MS indicated the formation of the pure product CT195. The product was used for the next step reaction without further purification. GC/MS: m/e 382 (65), 380 (100), 221 (33), 131 (33), 129 (39), 115 (32), 91 (37).

Synthesis of CT196. To a solution of CT195, prepared as indicated as above, in 5 mL of 1,4-dioxane and 1 mL of methanol was added 0.4 mL of NaOH solution (4.0 M), and the mixture was stirred at room temperature for 30 minutes. The mixture was diluted with hexane/dichloromethane (7/3), and washed with brine, dried (NaSO4) and concentrated. The crude product was purified on a silica gel column (packed with 1%TEA/hexance) eluted by 10-30% ethyl acetate in hexane to yield the desired product as yellow oil (0.37 g, 95% for the two steps, about 1:5 for a:b regional isomer). GC/MS: m/e 340 (63), 338 (100), 324 (25), 322 (40), 294 (21), 165 (18), 155 (16), 115 (20), 91 (21).

Synthesis of N244. To a solution of CT196 (1.0 g, 2.1 mmol.) in DMF (30 mL) was added NaH (60% on mineral oil, 168 mg, 4.2 mmol.), and the mixture was stirred at room temperature for 10 minutes. A solution of disuccinimidyl carbonate (1.6 g, 4.2 mmol.) in DMF (20 mL) was added to the reaction. The reaction was maintained at room temperature overnight. The mixture was concentrated, and was diluted in ethyl ether. The organic layer was extracted by water, dried over sodium sulfate and concentrated. The crude product was purified on a quick column of 50 g of silica gel. The column was packed in 1% TEA in dichloromethane (DCM) and was eluted by DCM to yield the desired product. The fractions were concentrated, and co-evaporated in acetonitrile to remove TEA and yield the desired product (0.50 g, 36%). The product is a mixture of two isomers, since the starting material is also a mixture of α and β substitutes. ¹H NMR (300 MHZ, CDCl₃) 5.28 (s, 1H), 4.98 (s, 1H), 4.63 (m, 1H), 4.49 (m, 2H), 4.40 (m, 2H), 4.30 (m, 4H), 4.08 (m, 2H), 2.43 (m, 2H), 2.02 (m, 2H); MS C₂₀H₁₉Cl₂FeNO₅ expected 479, found 480 (MH+).

Synthesis of N253. To a solution of N251 (1.0 g, 3.4 mmol.) in DMF (30 mL) was added NaH (60% on mineral oil, 274 mg, 6.84 mmol.), and the mixture was stirred at room temperature for 10 minutes. A solution of disuccinimidyl carbonate (2.63 g, 10.27 mmol.) in DMF (20 mL) was added to the reaction. The reaction was maintained at room temperature overnight. The mixture was concentrated, and was diluted in ethyl ether. The organic layer was extracted by water, dried over sodium sulfate and concentrated. The crude product was purified on a quick column of 50 g of silica gel. The column was packed in 1% TEA in dichloromethane (DCM) and was eluted by 50% DCM in hexane to yield the desired product. The fractions were concentrated, and co-evaporated in acetonitrile to remove TEA and yield the desired product (0.79 g, 53%). ¹H NMR (300 MHZ, CDCl₃) 4.33 (t, 2H), 4.29 (m, 2H), 4.14 (m, 2H), 4.09 (m, 2H), 4.00 (m, 2H), 2.84 (s, 4H), 2.40(t, 2H), 1.76 (m, 2H), 1.60 (m, 2H); MS C₁₉H₂₀ClFeNO₃ expected 433, found 433.

General procedure for the synthesis of ferrocene-DNA complexes. The DNA was dissolve in DI water, and the concentration was about 800 μM. The ferrocene derivatives were dissolved in DMF. The DNA solution (100 μL) was added by 200 μL of the ferrocene in DMF solution (50 eq.). The mixture was maintained at room temperature for over 8 hours. The sample was analyzed and purified by HPLC. The purified DNA-ferrocene complex was sent for MALDI-TOF mass analysis. MALDI-TOF data: expected for N239, 3261, found 3260; expected for N242: 3321, found 3317; expected for N245: 3341, found 3363 (M+Na⁺); expected for N254: 3295, found 3293.

### Example 5

### DNA Sequencing

The ferrocene labeled dideoxynucleotides with ferrocene derivatives prepared in Examples 14 will be used to label DNA fragments in chain termination sequencing.

The following experimental condition is designed for the demonstration only according to the routine chain termination sequencing procedure and optimal condition will be investigated. The M13 universal primer will be employed. The following solutions will be prepared: 5×Taq Mg Buffer (50 mM Tris Cl pH 8.5, 50 mM MgCl₂, 250 mM NaCl); Ferrocene-Terminator Mix (10-50 uM dGTP-Fc2, 10-50 uM dATP-Fc1, 10-50 uM dTTP-Fc4, and 10-50 uM dCTP-Fc3); and DNTP Mix (100 uM dGTP, 100 uM dATP, 100 uM dTTP, and 100 uM dCTP). The annealing reaction will carry out by combining in a microcentrifuge tube 3.6 ul of 5×Taq Mg Buffer, 0.4 pmol DNA template, 0.8 pmol primer, and water to a volume of 12.0 ul. The mixture will be incubated at 55°-65° C. for 5-10 minutes, cooled slowly over a 20-30 minute period to a temperature between 4°-20° C., then centrifuged once to collect condensation, mixed, and placed on ice. To the mixture is then added 1.0 ul dNTP Mix, 2.0 ul Ferrocene-Terminator Mix, 4 units of Taq polymerase, and water to bring the volume to 18.0 ul. The mixture is incubated for 30 minutes at 60° C., then placed on ice and combined with 25.0 ul of 10 mM EDTA pH 8.0 to quench the reaction. The DNA in the mixture is then purified in a spin column (e.g a 1 ml Sephadex G-50 column, such as a Select-D from 5 Prime to 3 Prime, West Chester, Pa.) and ethanol precipitated (by adding 4 ul 3M sodium acetate pH 5.2 and 120 ul 95% ethanol, incubating on ice for 10 minutes, centrifuging for 15 minutes, decanting and draining the supernatant, resuspending in 70% ethanol, vortexing, centrifuging for 15 minutes, decanting and draining the supernatant, and drying in a vacuum centrifuge for 5 minutes). The precipitated DNA is then resuspended in 3 ul of a solution consisting of 5 parts deionized formamide and 1 part 50 mM EDTA pH 8.0 and vortexed thoroughly. Prior to loading on the column, the mixture will be incubated at 90° C. for 2 minutes to denature the DNA.

### Example 6

### Ru2+ Based ETMs with Multiple Redox Potentials

Synthesis of Electrochemically-active Nucleotides and Tags

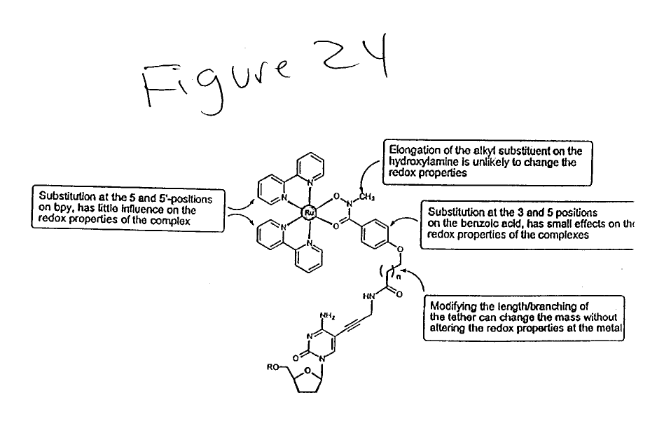

The synthetic approaches that will be utilized for the fabrication of electrochemically-active DNA tags are all well established. FIG. 21 illustrates the general retro-synthetic scheme. This scheme is highly convergent, and offers the opportunity to synthesize each fragment separately. Our approach will therefore include the synthesis of the following components: (a) bis-substituted Ru²⁺ precursors (R₂ bpy)₂RuCl₂, (b) substituted hydroxamic acids, bearing a functionalized linker, and (c) modified dideoxy nucleosides(tides). It is apparent that the approach is highly modular, as fragments can be easily modified and interchanged.

The synthesis of the Ru²⁺ precursors is easily achieved by reacting RuCl₃ with the desired substituted 2,2′-bipyridine or 1,10-phenanthorline ligands (Lay, P. A.; et al., Im Inorg. Synth. 1986, 24, 291-306, Shreeve, J. M. (Ed); John-Wiley & Sons, NY.; Bridgewater, et al., Inorg Chim. Acta 1993, 208, 179-188;. Struse, et al., Inorg. Chem. 1992, 31, 3004-3006). The c i s-(bpy)₂ is the thermodynamic product of this reaction. We routinely synthesize such building blocks in our laboratory (Tzalis, D.; et al., Inorg Chem., 1998, 37, 1121-1123). The substituted hydroxamic acids can be smoothly synthesized via the condensation reaction of commercially available protected hydroxylamines with substituted benzoic acids (Tor, Y.et al, J. Am. Chem. Soc. 1987,109, 6518-6519;. Libman, J.; et al., J. Am. Chem. Soc. 1987, 109, 5880-5881). Numerous benzoic acids are commercially available or are easily synthesized from accessible building blocks. The extended nucleosides are typically generated by Pd(0) mediated cross-coupling reactions between terminal alkynes (e.g., N-Boc-propargylamine) and 5-halo-pyrimidines or 7-halo-dazapurines. Such halogenated nucleosides are either commercially available or can be synthesized in one step from commercially available precursors(Yoshikawa, M.; et al., J. Org. Chem. 1969, 34, 1547-1550; Tzalis, D.; et al., Chem. Commun. 1996, 1043-1044; Tzalis, D.; et al., Angew. Chem. Int. Ed. Engl. 1997, 36, 2666-2668; Hurley, D.J.;etal., Chem. Commun. 1999, 993-994). In the last step, the modified nucleosides will be converted to their corresponding triphosphates using established procedures(Moffatt, I. G. Can. J. Chem. 1964, 42, 599-604; Slotin, L. A. Synthesis 1977, 737-75; Hutchinson, D. W. In Chemistry of Nucleosides and Nucleotides, L. B. Townsend, Ed., 1991, vol. 2, pp. 81-160) If complications arise, the nucleosides precursors can be converted into their monophosphate(,Yoshikawa, M.;et al., Bull. Chem. Soc. Jpn 1969, 42, 3505-3508; Imai, K.-I.;et al., J. Org. Chem. 1969, 34, 1547-1550) carried through additional synthetic steps, and converted to corresponding triphosphate in the very last step (Tor, Y.; et al. J. Am. Chem. Soc. 1993, 115, 4461-4467). Ion-exchanging chromatography using Sephadex A-25 and (Et₃Nh)⁺(HCO₃)⁻buffers will afford the desired novel nucleotides.

The phosphoramidites shown in FIG. 21 can be synthesized from the same Ru²⁺ precursors and similar hydroxamic acids that contain a hydroxyl group at the end of the linker. Phosphitylation using (2-cyanoethyoxy)-bis(diisopropylamino) phosphine in the presence of 1H-tetrazole provides the corresponding metal-modified phosphoramidites (Hurley, D. J.; et al., J. Am. Chem. Soc. 1998, 120, 2194-2195).

FIG. 20 depicts a representative retrosynthesis of an electrochemically-active nucleotide. Note that each fragment: the metal complex, the linker-containing hydroxamic acid, and the modified nucleoside(tide), can be separately synthesized. This makes the proposed approach extremely modular and versatile, and will allow us to tune the properties of the redox-active nucleotides.

Enzymatic Incorporation of Electrochemically-Active Nucleotides

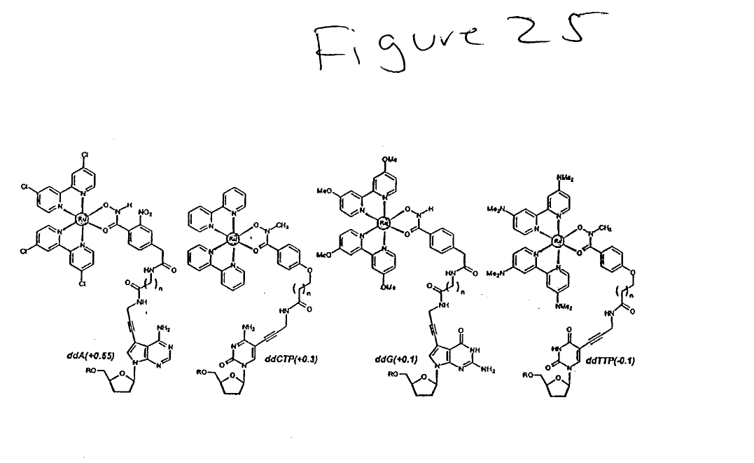

To evaluate the enzymatic incorporation of the novel metal-containing nucleotides, two major experiments will initially be conducted: (a) the enzymatic incorporation of modified dNTPs, and (b) the enzymatic incorporation of the corresponding ddNTPs (FIG. 22). The purpose of the first set of experiments will be to determine whether various polymerases can incorporate the modified deoxy nucleotides and continue elongation past the modification site. In this way we will be able to distinguish between chain termination that is caused by the inability of a polymerase to accept the modified dNTPs as substrates, and possible termination that occurs right after incorporation of the modified base. In the latter case, we will compare the sequencing lanes generated with the “natural” dideoxynucleotides to the lanes obtained with the redox-active dideoxynucleotides. Both experiments can utilize short, end-labeled primers that will be annealed to a longer DNA template.

In the first experiment, 4-individual templates that differ in their composition at a single position will be synthesized (FIG. 22a). The templates are designed to unequivocally determine if the incorporation of a specific nucleotide take place, and if full-length products are obtained. For example, experiment 1) in FIG. 6a, can be conducted with a 5′-labeled 13-mer primer. Primer extension in the presence of all four dNTPs will yield the full-length product. If dATP is eliminated, premature termination will occur right after the CGGC site yielding an 18-mer product. Shorter products will be easily separable from the full-length control product by PAGE. If the enzyme recognizes the modified deaza-A triphosphate as well as the resulting extended primer, addition of dATP(+0.55) will lead to the heneration of a full-lengh 22-mer product. If the enzyme can incorporate the modified base, but terminates right after incorporation, a 19-mer product will be obtained. If the modified triphosphate cannot serve as a substrate, an unmodified 18-mer will be obtained. Instead of using a 5′-labeled primer, information regarding the generation of a full length product can also be obtained by using the appropriate radiolabeled dNTP. For example, in experiment 1 (FIG. 22a), a full legnth radioactive band will only be observed if primer extension past the unique T takes place and if ³²P-dTTP is present in the reaction mixture. If dATP is replaced with dATP(+0.55) a full-length product is observed, we will be able to conclude that the enzyme recognizes the modified triphosphates and can continue polymerization past the modification site. Our observations will be fed back into the design and synthesis of second-generation redox-active nucleotides.

In the second experiment, dideoxy Sanger sequencing will be investigated where the behavior of the modified triphosphates will be compared to their “native ddNTPs (FIG. 22b).³³ In this case, a longer DNA template will be used (typically a plasmid fragment). T7 DNA Polymerase will initially be used for the Sanger sequencing experiments using published conditions. Alternative enzymes (e.g., Thermosequenance or AmpliTaq DNA ploymerases) and modified conditions will be explored at more advanced stages.⁴¹

Optimization of the Electrophoretic Behavior of Redox-Active Nucleotides.

Two optimization procedures will be addressed: (a) optimizing the enzymatic incorporation of the modified nucleotides as discussed above, and (b) optimizing the electrophoretic mobility of the modified nucleotides. While these can be viewed as tow separate processes, they are interrelated. The structure (mass) and charge of the electroactive moiety, tethered to the nucleobase, influence both its recognition by the enzyme, and its electrophoretic behavior. It is highly likely that the modified nucleotides will be accepted as alternative substrates by the various polymerases, since the structurally-related fluorescently-tagged nucleotides are all well-behaved. Hence, fine-tuning of the electrophoretic mobility will h ave to be addressed to ensure reliable correlation between the electrophoretic band-positioning and base identity.

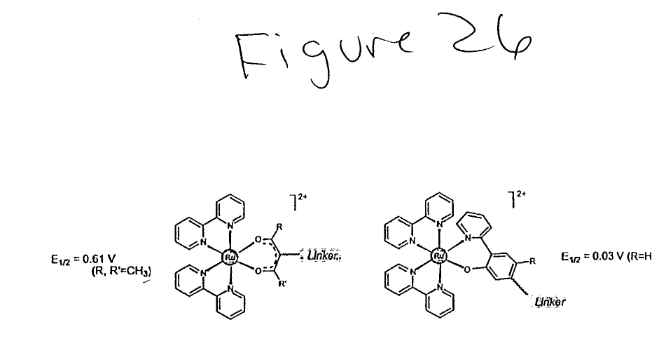

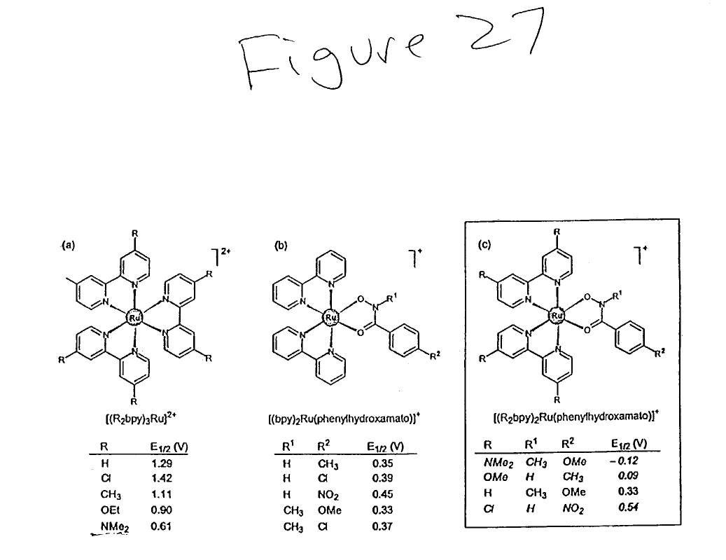

FIG. 23 depicts various positions are suitable for structural modifications without altering the electrochemical propitious of the metal center.

Incorporation of metal-containing nucleotides into the DNA chain will result in fragments that will display slower electrophoretic migration when compared to their corresponding native fragments. This is due to the increased mass and additional single positive charge at the metal center. Since we intend to use structurally-related redox active moities (see FIG. 24), we anticipate that by changing the linkers and the introduction of “siclent” substituations (as illustrated in FIG. 23), we will be able to bring the various nucleotides to display very similar “electrophoretic behavior. Similar consideration have been applied for the generation of “electrophoretically-uniform” flourescent dyes for current automated DNA sequencing.

Experimentally, the electrophoretic behavior of the various ddNTPs will be investigated using the general scheme shown in FIG. 22B. Sanger sequencing of a long DNA template will be conductive and the relative migration of all the modified ddNTPs will be correlated. Based on the observed relative migration, synthetic modification will be incorporated into the design of our second generation redox-active nucleotides.

Alternative Designs

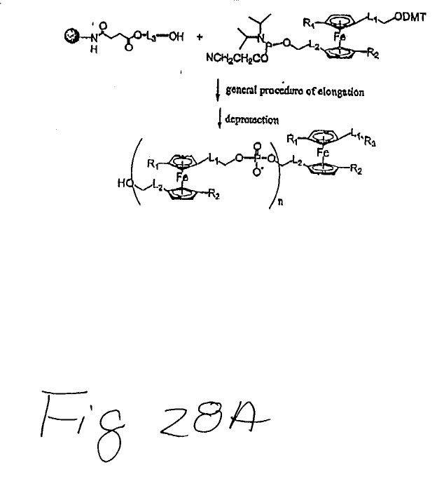

It is important to emphasize that alternative structures for redox active proves do exist and will be considered if complications arise with the system discussed above. Two selected examples are shown in FIG. 8, where alternative negatively charged ligands are coordinated to a [(bpy)₂Ru]²⁺ core. The parent unsubstituted derivatives exhibit a reversible metal-centered Ru^{2+,3+} wave either close to or within the operative range we defined above (Juris, A.; et al., Coord. Chem. Rev. 1988, 84, 85-277; Tabor, S.; et al., Proc. Natl. Acad. Sci. USA 1987, 84, 4767-4771). One of the most versatile system is the acetylacetonato ligand, as the electron density on the anion can be controlled by the flanking substituents. The introduction of appropriate substitutions will therefore allow us to tune these redox potentials. Synthetically, various tethers can be easily connected to the 2-position. Treating the 1,3-diketone precusor with base will afford a stable enolate that can be easily alkylated with a suitable functionalized electrophile (e.g., protected 6-bromehexanoic acid). By following analogous retrosynthetic analysis as shown above (FIG. 21), these complexes can be conjugated to the extended nucleosides to afford alternative ddNTPs. Similaryly, the redox potential of the complexes derived from the hydroxyphenly-pyridyl system can be tune by the appropriate substation. FIG. 25 illustrates two alternative designs for tunable redox-active centers that can be linked to modified ddNTP's (see ref. 30 and 44 for electrochemical information).

Electrochemical Detection of Redox-Active Oligonucleotides

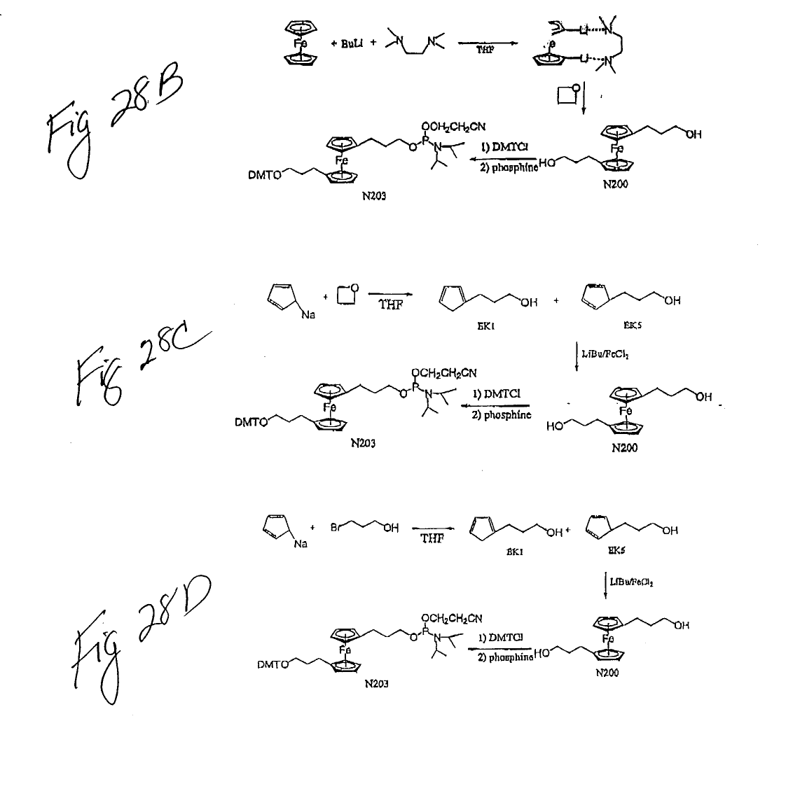

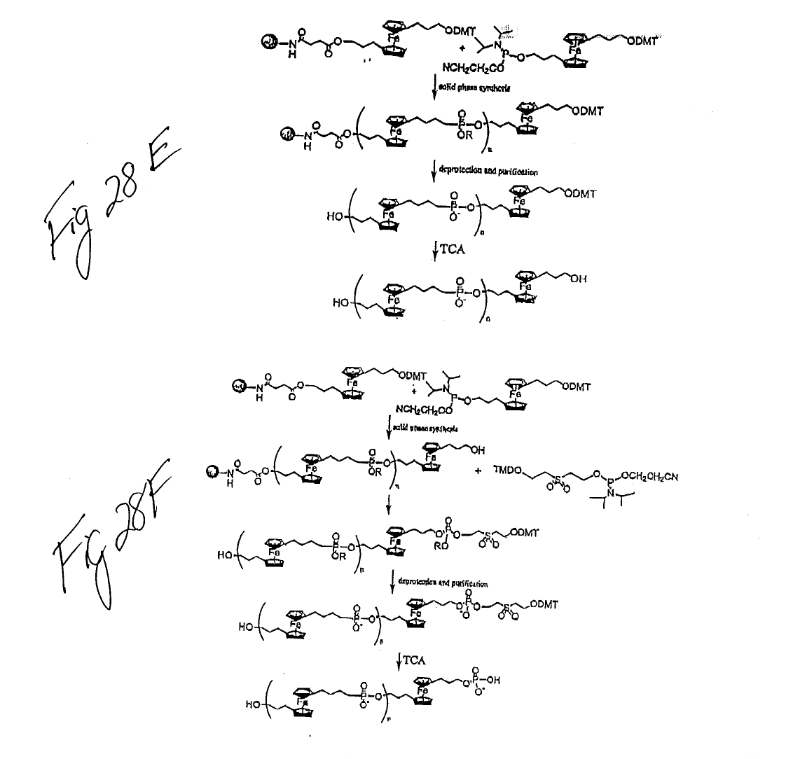

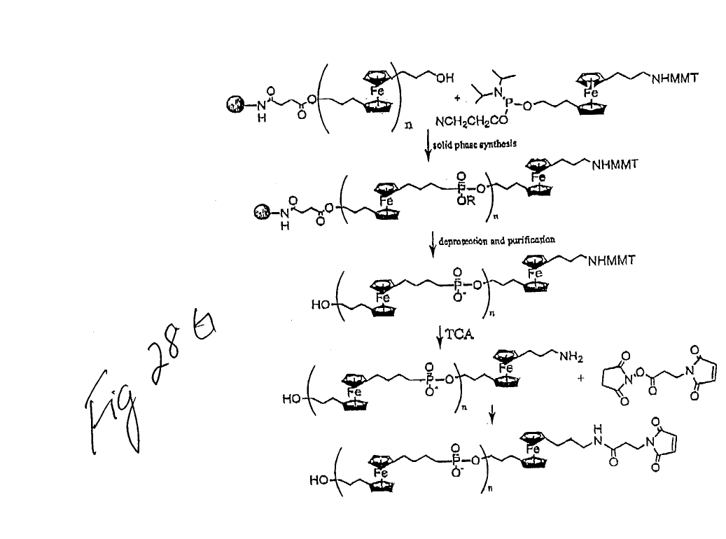

All redox active compounds prepared will be analyzed in our laboratory using cyclic and square-wave voltammentry. We routinely use these techniques to characterize metal complexes. We will first characterize the electrochemical characteristics of the new [(byp)₂Ru(L⁻)]⁺ complexes (FIG. 26c). This will be followed by the electrochemical characterization of the metal-containing nucleosides (FIG. 24) to verify that conjugation does not alter their redox behavior. We will then prepare short oligonucleotides that are tagged with redox active moieties at their 5′-end by using the phosphoramidites shown in FIG. 21. Voltammentry techniques will then be applied to detect the presence of electrochemically-active oligonucleotide on the surface. This system will be used to define the lower limit of detection and to explore potential electrochemical techniques that can enhance sensitivity and lower the limit of detection.

All references are incorporated by reference, as well as U.S. Ser. No. 09/626,096, filed Jul. 26, 2000 and WO 01/07665. A-70476-2 1/41 xj (v) nA xj (v) nA xi (v) nA A-70476-2 2 /41 i i I I I O a) a) a) a) a) a) a) a) aD E>C D LL A-70476-2 3 3/41 means and stdev 1 &|1.0000 [@z|0.0000 Ip 1p @hE |13.0860 ||0.2820 || al 1p &|0 0000 1ff110.0039 || eO(v) 1p p|13.0860 |ffia0.2820 a12p p|0.1323 1ff110.0039 || eO(v) 2p @p|13.8680 |ffia0.2820 a13p 710 3070 1ffF0.0039 eO(v) 3p p|13.8680 |pfi|0a2820 a14p &10 5000 1ff110 0039 eO(v) 4p FIG. _{—}6 A-70476-2 4/41 N c, >A-70476-2 5 /41 a O a O a O aO 00>>C-A-70476-2 6 /41 A-70476-2 7/ 41

Ci c CL 0 * A-70476-2 8/41 C) A-70476-2+9/41 C” (v) dI A-70476-2 10 /41 A-70476-2 11 /41 Fe⁺ Fe>Fe <h~tBuLi <d><ffi>dibronmopropane NAOH~ PS26 PS27 O Br Fe PS31+HOt OH DMTCI HO ODMT OH pyridine ODMT K158 NaH/DMF TCA Fe ODMT CHE d CT168 O 0 u ODMT Fe <phosphine CT169 geagent Fo O0_p/OCH₂CH₂CN Fe ⁰P CT170 FIG. _{—}14 A-70476-2+12 /41

Br Fe⁺ nBuLi+H₃C SSO₂Br T Fe THF B N225 Br K1 64 0 Cu(OAc)₂ AcO Br+Fe BrCOR AcO AcO - <AIC[₃ Fe N227 __AcO MgCl2 /Zn methanol CT46 AcO - Br MeO __Br MeI/ NaOH Fe Fe AcO - MeO N219 N224 7 NaH/[₀ ODMT MeO Z~stODMT ODMT ODMT

FeeO N228 MeO o ODMT Fe OH Fe MeO phosphine reagent N229 DMT MeO OCH₂CH₂CN MeO N230 FIG._{—}15 A-70476-2+13/41

B(OR)₂<Br T 1) tBuLi (1 eq) B(R₂B Fe 2) Tributylborate (1 2 eq) Fe CuBr₂ (excess) THF, -78° C. water/ MeOH/toluene CK71 500-70° C. CT45

Cl DCM, 0° C. (80%) Br TiCl₄ (1.0 eq) Br Et₃SiHI (4 eq) Fe Fe DCM, 16 hours B r (80%) Br S16 CT160 FIG. 16A Br+DMTO ODMT OH K158 Fe Br Na SJ6 ₀ODMT ODMT

Fe ITCA (1 eq) Br 1DCM SJ7 ODMT Fe phosphine <~Br reagent SJ8 DCM (˜85%)

ODMT Fe 0 Br NCI SJ9 FIG. _{—}16B A-70476-2 14/41 Fe 1) tBuLi/THF Fe 2 Fe 2) B(OBu)₃,OH H₂O, 65° C. %OH CK71 CK73

CICOR AIC13 Br ~;Br HgCl2/Zn Fe water toluene/HCl Fe N248 N247 Br DMTO ODMT+OH Fe K1 58 N248 DMF 0 ODMT ODMT Fe TCA(1 eq) Cl DCM SJ59 (˜50%) Fe phosphine Ci reagent SJ61 DCM (87%)

ODMT Fe/>SJ63 FIG. 16C A-70476-2 15/41 0 0 AICI₃ rBr Fe⁺ CI CH₂C₂ R.T Fe Nl Zn, HgCl₂ I Toluene, R.T.

Br Fe HO{ODMT NaH, DMF, R.T.

ODMH overnight K158 ODMT ODMT Fe >~1) TCA, DCM, R.T. ₂) TEAA/ MeOH, K159+DCM, R.T. Fe 4<i J CN K160 NplO C96, DCM, 0° C. YN I 0_CODMT Fe F 1761 0 CN FIG₅~ 17A A-70476-2 16/41+BrH THF OH Na EK1 EK5 LiBu/FeCl₂ OH Fe HO O N200 DMTCI OH Fe DMTO N₂O₃ phosphine OCH₂CH₂CN Fe DM10 N₂O₄ FIG. _{—}17B A-70476-2 17/41 Fe⁺ nBuLi+H₃C 0 | THF Fe N225 CT184 BrCOR AICl₃+HCl IHgCI₂ IZn ci ~ SJ30 ci ~ CT1 85HO™ OMT DMTCI HO ~OH INaH /DMF 0ODM™ CI~ phosphine reagent ci ODMT OCH₂CH₂C Fe 0p A-70476-2 18 /41 0 Br Fe⁺ nBuLi+H₃C S S—Br - >Fe N225 BRCOR K64 AIC13 Br Br Br Br Fe ~HCl/HgCl₂/Zn F Br N221 Bra CT176 HO ODMT DMTCI HO OH ODMT -OH Kl 58 NaH DMF Bra<0 ODMT Fe ODMT Br TBATB methanol Br 0 ODMT Fe OH Br phosphine 4 reagent Br 0ODM™ Br sN FIG._{—}18B A-70476-2 19/41 0 0 Fe CH₂Cl₂ Fe 0 0 CT1 50H HO NH₂ CH₂Cl₂ N o0 p N H Fe I N NOH Fe171 (phosphane >CT171)-CN CH₂Cl₂ Fe CT1 51 FIG. _{—}18C 0 CH₂ Cl r o Br CH₂₁₂ Cl Br Zn/TiCl₄ Fe Fe Cl ~ >CT185 Cl CT186 NaH D ODMT Ci O DMT ODMT Fe ODMT CH H₂ methan Fe OH Ci I phosphine reagent A-70476-2+20 /41 Fe⁺ tBuLi+12 Fe PS26 ,OH lPd DIPEA/ THF Br OH Fe Ph₃P/CBr₄ Fe SJ16 SJ12 HO ODMT (1) DMTCI HO OH OTBS (2) TBSCI OH SJ11 NaH/DMF 4/MODMT Fe SJ17 MT Fe phosphine SJ18 reagent ODMT 0-NOCH₂CH₂CN SJ21 FIG. _{—}18E A-70476-2+21/41

R1ffim Br KOAC DMF R₁ Br Fe Fe R₂, R₂ N219 R1=R2=OCH₃, X═H R1=R2=OCH₃, X═H N2 R1=R2=X═H CT193 R1=R2=X═H N251 R1=H, R2=CI, X═H R1=H, R2=CI, X═H CT186 R1=R2=CI, X═CH CT195 R1=R2=CI, X═CH

I NaOH, 75% R₁ 0oo 0-N R₁ OH Fe 0 O Fe R₂, R₂ N243 R1=R2=OCH₃, X═H N235 R1=R2=OCH₃, X═H N238 R1=R2=X═H^{C}_{T1}⁹⁴ R1=R2=X═H N253 R1=H, R2=CI, X═H 1) NaH, DMN248 R1=H, R2=CI, X═H N244 R1=R2=CI, X═CH 2) Disuccinimyl CT196 R1=R2=CI, X═CH carbonate 50% R_{10 0}- +NH₂-TTTTTTTTTT-OH

Fe 0 0 R2>

DMF/ buffer, pH=7 N243 R1=R2=OCH₃, X═H rt, 8 hrs. N238 R1=R2=X═H+N253 R1=H, R2=CI, X═H N244 R1=R2=CI, X═CH X X H 5′ 3′ R₁ 0N-TTTTTTTTTT- OH

Fe 0 N242 R1=R2=OCH₃, X═H N239 R1=R2=X═H R 2>N254 R1=H, R2=CI, X═H

N245 R1=R2=CI, X═CH FIG._{—}19 A-70476-2 22/41 0 0 0 0.2 0=-0 0 0 o w=0 0 0 0 A-70476-2 23/41 General Structure for Electrochemical Sequencing: Base Attachment: ETM I L NH9 0 0 0N I I I I I I -O—P—O—P—O—P-0 I II DDATP-ETM ETM

L 0 0 0 0H ° II I INH -O—P—O—P—P-0>NH2 DDGTP-ETM ETM

ET| NH₂ 0. 0 0 I I II II -O—P—O—P—O—P-° DDCTP-ETM ETM O 0 0 NH -0o-P-o-P-o-P-o0 NA DDUTP-ETM

FIG. _{—}20B A-70476-2 24/41 m cq I z 0 0 0 I+- _cZ-I zz 0 A-70476-2 25/41 Ci NMe2 Me2NX OMe ol sN CH3 FIG. -22 A-70476-2 26/ 41 E x co HZ a) a) E E U U E4 EE U U E1 E U U0 Ln c Ln cn Lnm Lnm A-70476-2+27/41 z z z-0 V- a) a C a)~ 0 a) a A-70476-2+Cl 28/41 Ci O )ₙ HN 0 NH₂+0dCTP(+O.³) ddA(+0.55)/EN ROLES OMe /NMe₂ MeO NJ Me₂N N Meo N Me₂N ( OMe 0)ₙ NMe₂ HN 0 NH ° ddG(+O.) NH R0 A-70476-2 29 /41 E₁/₂=0.61 V (R, R=CH₃) 2+

Linker E₁/₂=0.03 V (R═H) FIG. -26 A-70476-2+30/41 cc+A-70476-2 31/41+y R1 L1 LODMT H 0 p-2 R2 NCH₂CH₂CO

General Procedure of Elongation Deprotection R₁₃ (R₁ L₁ 0OOYO L₂ R₂ Fe O—HO R₂ n R FIG. -28A Li———-N Fe⁺ BuLi+;N Nj THF1 Fe OH Fe HO N200 1) DMTCI 2) phosphine OCH₂CH₂CN DMTO N₂O₃ FIG. _{—}28B A-70476-2 32 /41 C+THF CmOH Na EK1 EK5 sOH Lu/FeCl₂ Fe HOO N200 1) DMTCI 2) phosphine OCH₂CH₂CN Fe DMTO™ N₂O₃ FIG._{—}28C +Br OH THF+Na EK1 EK5 HOH %(U I FeCl₂ Fe HO N200 1)DMTCI {12) phosphine OCH₂CH₂CN Fe DMTO N₂O₃ FIG._{—}28D A-70476-2 33 /41 O-ODMT ODMT vHNW 0NCH₂CH₂CO Solid Phase Synthesis ODMT 0 Fe p-O H 0 Deprotection and Purification ODMT 0 Fe II Fe u-HO ITCA OH

K Fe II Fe 0-HO FIG.-28E A-70476-2 34/41<ODMT Y ODMT 0 Fe+)N p Fe NH NCH₂CH₂CO O Solid Phase Synthesis OH 0 Fe II 0 t Fe OR H n+TMDO S₅ olp,OCH₂CH₂CN //S I 0 Fe OU HO

Deprotection and Purification 0 HO Fe TCA <Io-P—OH p-O Fe HO

FIGi,28F A A-70476-2+35/41 OH NHMMT 0 Fe⁺ Fe ° H n NCH₂CH₂CO Solid Phase Synthesis +<IMNHMMT O Fe Deprotection and Purification II Fe U HO Fe O HO-I 0 Fe p-0 Fe HO FIGN228G A-70476-2 36 /41 0 U) 0I N u A-70476-2 37/41 a) oo O═CL~O I A-70476-2 38 /41 lO,R_Fc 3}NH L- Fc 0-O—R-Fc>}NH L- Fc NaBH₄H₂ CH—R-Fc+LmNH -C-L- Fc ═N—R-Fc NH-.N-L- Fc H DNA oligos Fc ferrocene derivatives FIG.-29B A-70476-2 39/41 Comp. Structure Ep (mV) MeO S₂O₃Na N220 Fe 0 MeO SJ33 Fe 170 SJ28 LOP-OH 320

Fe OH SO₃Na SJ9 Fe 360 Bra SJ37 , 360 N249 Fe 350 Br S₂O3Na SJ31 Fe 470 Bra SJ32 Fe 480 cl -<FSO₃Na CT187 520 Fe FIG.-30 A-70476-2 40/41 dC/A/G>+dT+dT -dT -dT C T -dRu +dRu +dRu -dRu T G A G G G 13-mer FIG. _{—}31 A-70476-2 41/41 Capillary Electrophoresis Channel (1) 0.12V 0.50V——-————->.n Li -, Li Electrode —O.1V 0.27V FIG. _{—}32A (1)3 (>>[ ] Electrode +0.65VA 0.25V I V V FIG. -32B n FIG._{—}32C FIG. -32D

We claim:

1. A composition comprising:
a) a first nucleic acid comprising a first ETM with a first redox potential; and
b) a second nucleic acid comprising a second ETM with a second redox potential;
c) a third nucleic acid comprising a third ETM with a third redox potential; and
d) a fourth nucleic acid comprising a fourth ETM with a fourth redox potential, wherein said first, second, third and fourth redox potentials are different.

2. A composition according to claim 1 wherein the sequences of said first, second, third and fourth nucleic acids are different.

3. A composition according to claim 2 wherein the sequence of first, second, third and fourth nucleic acids differ by only one base.

4. A composition according to claim 3 wherein the nucleoside comprising the different base comprises said ETM.

5. A composition according to claim 1 wherein said nucleic acids are single stranded.

6. A composition according to claim 1 wherein at least one of said ETMs is a transition metal complex.

7. A composition according to claim 6 wherein said transition metal complex is ferrocene.

8. A composition according to claim 1 wherein all of said ETMs are ferrocene derivatives. S

9. A composition according to claim 6 wherein at least one of said transition metal complexes is a ruthenium complex.

10. A composition according to claim 6 wherein all of said ETMs are ruthenium derivatives.

11. A method of determining the identification of a nucleotide at a detection position in a target sequence, wherein said target sequence comprises a first target domain directly 5′ adjacent to said detection position, said method comprising:
a) providing a first hybridization complex comprising said target sequence and an extension primer hybridized to said first target domain of said target sequence;
b) contacting said hybridization complex with:
i) a polymerase enzyme; and
ii) a composition comprising a plurality of chain terminating NTPs each comprising a covalently attached ETM, each NTP comprising an ETM with a different redox potential;
under conditions whereby if one of said NTPs basepairs with the base at said detection position, said extension primer is extended by said enzyme to incorporate said ETM and form an extended primer; and
c) identifying the base at said detection position.

12. A method according to claim 11 wherein said identification step comprises:
a) contacting said extended primer with a solid support comprising an array of electrodes comprising capture probes to form second hybridization complexes;
b) applying an input signal to said electrodes; and
c) detecting an output signal characteristic of said ETM.

13. A method according to claim 11 wherein said extension primer is attached to an electrode on a solid support.

14. A method according to claim 13 wherein said identification step comprises:
a) applying an input signal to said electrodes; and
b) detecting an output signal characteristic of said ETM

15. A method of determining the identification of a nucleotide at a detection position in a target sequence comprising:
a) providing a solid support comprising an array of electrodes each comprising a capture probe;
b) contacting said array with a plurality of detection probes each comprising:
i) a unique nucleotide at the interrogation position; and
ii) an ETM with a unique redox potential; and
c) detecting a signal from at least one of said ETMs to identify the nucleotide at the detection position.

16. A method of sequencing a target nucleic acid comprising:
a) providing a plurality of sequencing probes complementary to said target sequence, each of a different length, each comprising a different chain terminating NTP comprising an ETM comprising a different redox potential;
b) separating said nucleic acids on the basis of size; and
c) detecting each of said ETMs to identify the sequence of at least a portion of said target nucleic acid.

17. A method of making a plurality of sequencing probes, each with a covalently attached ETM with a different redox potential, said method comprising:
a) providing a first oligonucleotide substituted with a first 5′ protected deoxynucleotide;
b) providing a first ETM derivative with a first redox potential;
c) mixing said first oligonucleotide with said first ETM derivative to form a first sequencing probe with a first deoxynucleotide triphosphate comprising a first ETM with a first redox potential;
d) providing a second oligonucleotide substituted with a second 5′ protected deoxynucleotide;
e) providing a second ETM derivative with a second redox potential;
f) mixing said second oligonucleotide with said second ETM derivative to form a second sequencing probe with a second deoxynucleotide triphosphate comprising a second ETM with a second redox potential

18. A method according to claim 17 further comprising:
a) providing a third oligonucleotide substituted with a third 5′ protected deoxynucleotide;
b) providing a third ETM derivative with a third redox potential; and
c) mixing said third oligonucleotide with said third ETM derivative to form a third sequencing probe with a third deoxynucleotide triphosphate comprising a third ETM with a third redox potential.

19. A method according to claim 18 further comprising:
a) providing a fourth oligonucleotide substituted with a fourth 5′ protected deoxynucleotide;
b) providing a fourth ETM derivative with a fourth redox potential; and
c) mixing said fourth oligonucleotide with said fourth ETM derivative to form a fourth sequencing probe with a fourth deoxynucleotide triphosphate comprising a fourth ETM with a fourth redox potential.

20. A method according to claims 17, 18, and 19 wherein said first, second, third and fourth deoxynucleotide triphosphates are different.

21. A composition according to claims 17, 18, and 19 wherein at least one of said ETMs is a transition metal complex.

22. A composition according to claim 21 wherein said transition metal complex is ferrocene.

23. A method according to claim 21 wherein said detecting comprises passing said sequencing probes over four sequential electrodes comprising different potentials.

24. A method according to claim 21 wherein said detecting comprises passing said sequencing probes over a single electrode.

25. A method of making a plurality of nucleic acids, each with a covalently attached ETM with a different redox potential, said method comprising:
a) providing a first transitional metal complex with a first redox potential and a first functional group;
b) providing a first oligonucleotide substituted with a second functional group; and
c) mixing said first transition metal complex with said first oligonucleotide to form a first transition metal complex-oligonucleotide conjugate with a first redox potential;
d) providing a second transitional metal complex with a second redox potential and a first functional group;
b) providing a second oligonucleotide substituted with a second functional group; and
c) mixing said second transition metal complex with said second oligonucleotide to form a second transition metal complex-oligonucleotide conjugate with a second redox potential.
